SEQUENCE LISTING (183 sequences)

SEQ ID NO: 1 - protein, 213 aa, synthetic construct
  REGION 1..213: note = Synthetic
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 451 aa, synthetic construct
  REGION 1..451: note = Synthetic
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 219 aa, synthetic construct
  REGION 1..219: note = Synthetic
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, synthetic construct
  REGION 1..449: note = Synthetic
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, synthetic construct [sequence omitted]

SEQ ID NO: 6 - protein, 448 aa, synthetic construct
  REGION 1..448: note = Synthetic
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 10 - protein, 333 aa, Homo sapiens [sequence omitted]

SEQ ID NO: 11 - protein, 9 aa, synthetic construct
  REGION 1..9: note = Synthetic
YCQQWTSNP

SEQ ID NO: 12 - protein, 9 aa, synthetic construct
  REGION 1..9: note = Synthetic
PKSCDKTHT

SEQ ID NO: 13 - protein, 9 aa, synthetic construct
  REGION 1..9: note = Synthetic
ASVKMSCKA

SEQ ID NO: 14 - protein, 9 aa, synthetic construct
  REGION 1..9: note = Synthetic
LLGGPSVFL

SEQ ID NO: 15 - protein, 8 aa, synthetic construct
  REGION 1..8: note = Synthetic
LLGGPSVF

SEQ ID NO: 16 - protein, 9 aa, synthetic construct
  REGION 1..9: note = Synthetic
KKAEPKSCD

SEQ ID NO: 17 - protein, 8 aa, synthetic construct
  REGION 1..8: note = Synthetic
KEPKSCDK

SEQ ID NO: 18 - protein, 9 aa, synthetic construct
  REGION 1..9: note = Synthetic
AEPKSCDKT

SEQ ID NO: 19 - protein, 9 aa, synthetic construct
  REGION 1..9: note = Synthetic
EPKSCDKTH

SEQ ID NO: 20 - protein, 9 aa, synthetic construct
  REGION 1..9: note = Synthetic
KSCDKTHTC

SEQ ID NO: 21 - protein, 111 aa, synthetic construct
  REGION 1..111: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVA

SEQ ID NO: 22 - protein, 214 aa, synthetic construct; identical to SEQ ID NO: 7
  REGION 1..214: note = Synthetic

SEQ ID NO: 23 - protein, 240 aa, synthetic construct
  REGION 1..240: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGG

SEQ ID NO: 24 - protein, 8 aa, synthetic construct
  REGION 1..8: note = Synthetic
PAPELLGG

SEQ ID NO: 25 - protein, 11 aa, synthetic construct
  REGION 1..11: note = Synthetic
PAPELLGGPSV

SEQ ID NO: 26 - protein, 25 aa, synthetic construct
  REGION 1..25: note = Synthetic
NVNHKPSNTKVDKKVEPKSCDKTHT

SEQ ID NO: 27 - protein, 20 aa, synthetic construct
  REGION 1..20: note = Synthetic
EVTHQGLSSPVTKSFNRGEC

SEQ ID NO: 28 - protein, 11 aa, synthetic construct
  REGION 1..11: note = Synthetic
GGPSVFLFPPK

SEQ ID NO: 29 - protein, 10 aa, synthetic construct
  REGION 1..10: note = Synthetic
YYCSRWGGDF

SEQ ID NO: 30 - protein, 5 aa, synthetic construct
  REGION 1..5: note = Synthetic
RTVAA

SEQ ID NO: 31 - protein, 14 aa, synthetic construct
  REGION 1..14: note = Synthetic
CPPCPAPELLGGPS

SEQ ID NO: 32 - protein, 9 aa, synthetic construct
  REGION 1..9: note = Synthetic
AATYYCQQW

SEQ ID NO: 33 - protein, 9 aa, synthetic construct
  REGION 1..9: note = Synthetic
KKAEPKSCD

SEQ ID NO: 34 - protein, 9 aa, synthetic construct
  REGION 1..9: note = Synthetic
KAEPKSCDK

SEQ ID NO: 35 - protein, 9 aa, synthetic construct
  REGION 1..9: note = Synthetic
AEPKSCDKT

SEQ ID NO: 36 - protein, 9 aa, synthetic construct
  REGION 1..9: note = Synthetic
EPKSCDKTH

SEQ ID NO: 37 - protein, 9 aa, synthetic construct
  REGION 1..9: note = Synthetic
KSCDKTHTC

SEQ ID NO: 38 - protein, 11 aa, synthetic construct
  REGION 1..11: note = Synthetic
PELLGGPSVFL

SEQ ID NO: 39 - protein, 10 aa, synthetic construct
  REGION 1..10: note = Synthetic
PELLGGPSVF

SEQ ID NO: 40 - protein, 6 aa, synthetic construct
  REGION 1..6: note = Synthetic
SLRLSC

SEQ ID NO: 41 - protein, 7 aa, synthetic construct
  REGION 1..7: note = Synthetic
SRWGGDG

SEQ ID NO: 42 - protein, 6 aa, synthetic construct
  REGION 1..6: note = Synthetic
PGGSLR

SEQ ID NO: 43 - protein, 6 aa, synthetic construct
  REGION 1..6: note = Synthetic
SWRGGD

SEQ ID NO: 44 - protein, 5 aa, synthetic construct
  REGION 1..5: note = Synthetic
SRWGG

SEQ ID NO: 45 - protein, 7 aa, synthetic construct
  REGION 1..7: note = Synthetic
NVNHKPS

SEQ ID NO: 46 - protein, 6 aa, synthetic construct
  REGION 1..6: note = Synthetic
SGGGLV

SEQ ID NO: 47 - protein, 6 aa, synthetic construct
  REGION 1..6: note = Synthetic
QGTLVT

SEQ ID NO: 48 - protein, 6 aa, synthetic construct
  REGION 1..6: note = Synthetic
GGLVQP

SEQ ID NO: 49 - protein, 6 aa, synthetic construct
  REGION 1..6: note = Synthetic
LVTVSS

SEQ ID NO: 50 - protein, 5 aa, synthetic construct
  REGION 1..5: note = Synthetic
SRWGG

SEQ ID NO: 51 - protein, 6 aa, synthetic construct
  REGION 1..6: note = Synthetic
KKVEPK

SEQ ID NO: 52 - protein, 6 aa, synthetic construct
  REGION 1..6: note = Synthetic
PAPIEK

SEQ ID NO: 53 - protein, 5 aa, synthetic construct
  REGION 1..5: note = Synthetic
SLSPG

SEQ ID NO: 54 - protein, 6 aa, synthetic construct
  REGION 1..6: note = Synthetic
VSNKAL

SEQ ID NO: 55 - protein, 5 aa, synthetic construct
  REGION 1..5: note = Synthetic
SLSPG

SEQ ID NO: 56 - protein, 7 aa, synthetic construct
  REGION 1..7: note = Synthetic
REEMTKN

SEQ ID NO: 57 - protein, 7 aa, synthetic construct
  REGION 1..7: note = Synthetic
HYTQKSL

SEQ ID NO: 58 - protein, 6 aa, synthetic construct
  REGION 1..6: note = Synthetic
KVSNKA

SEQ ID NO: 59 - protein, 5 aa, synthetic construct
  REGION 1..5: note = Synthetic
SLSPG

SEQ ID NO: 60 - protein, 8 aa, synthetic construct
  REGION 1..8: note = Synthetic
EYKCKVSN

SEQ ID NO: 61 - protein, 5 aa, synthetic construct
  REGION 1..5: note = Synthetic
SLSPG

SEQ ID NO: 62 - protein, 5 aa, synthetic construct
  REGION 1..5: note = Synthetic
SRTPE

SEQ ID NO: 63 - protein, 6 aa, synthetic construct
  REGION 1..6: note = Synthetic
HEALHN

SEQ ID NO: 64 - protein, 5 aa, synthetic construct
  REGION 1..5: note = Synthetic
QMTQS

SEQ ID NO: 65 - protein, 6 aa, synthetic construct
  REGION 1..6: note = Synthetic
PSDEQL

SEQ ID NO: 66 - protein, 5 aa, synthetic construct
  REGION 1..5: note = Synthetic
VTITC

SEQ ID NO: 67 - protein, 5 aa, synthetic construct
  REGION 1..5: note = Synthetic
LSSPV

SEQ ID NO: 68 - protein, 6 aa, synthetic construct
  REGION 1..6: note = Synthetic
DIQMTQ

SEQ ID NO: 69 - protein, 6 aa, synthetic construct
  REGION 1..6: note = Synthetic
KRTVAA

SEQ ID NO: 70 - protein, 6 aa, synthetic construct
  REGION 1..6: note = Synthetic
SLRLSC

SEQ ID NO: 71 - protein, 7 aa, synthetic construct
  REGION 1..7: note = Synthetic
SSASTKG

SEQ ID NO: 72 - protein, 5 aa, synthetic construct
  REGION 1..5: note = Synthetic
GDRVT

SEQ ID NO: 73 - protein, 5 aa, synthetic construct
  REGION 1..5: note = Synthetic
GQGTK

SEQ ID NO: 74 - protein, 6 aa, synthetic construct
  REGION 1..6: note = Synthetic
APSVFI

SEQ ID NO: 75 - protein, 6 aa, synthetic construct
  REGION 1..6: note = Synthetic
GTLVTV

SEQ ID NO: 76 - protein, 6 aa, synthetic construct
  REGION 1..6: note = Synthetic
CNVNHK

SEQ ID NO: 77 - protein, 6 aa, synthetic construct
  REGION 1..6: note = Synthetic
SKSTSG

SEQ ID NO: 78 - protein, 7 aa, synthetic construct
  REGION 1..7: note = Synthetic
NVNHKPS

SEQ ID NO: 79 - protein, 5 aa, synthetic construct
  REGION 1..5: note = Synthetic
PSVFI

SEQ ID NO: 80 - protein, 7 aa, synthetic construct
  REGION 1..7: note = Synthetic
VYACEVT

SEQ ID NO: 81 - protein, 9 aa, synthetic construct
  REGION 1..9: note = Synthetic
PAPELLGGP

SEQ ID NO: 82 - protein, 13 aa, synthetic construct
  REGION 1..13: note = Synthetic
PAPELLGGPSVFL

SEQ ID NO: 83 - protein, 8 aa, synthetic construct
  REGION 1..8: note = Synthetic
ELLGGPSV

SEQ ID NO: 84 - protein, 9 aa, synthetic construct
  REGION 1..9: note = Synthetic
GGPSVFLFP

SEQ ID NO: 85 - protein, 8 aa, synthetic construct
  REGION 1..8: note = Synthetic
ELLGGPSV

SEQ ID NO: 86 - protein, 11 aa, synthetic construct
  REGION 1..11: note = Synthetic
GGPSVFLFPPK

SEQ ID NO: 87 - protein, 8 aa, synthetic construct
  REGION 1..8: note = Synthetic
PAPELLGG

SEQ ID NO: 88 - protein, 11 aa, synthetic construct
  REGION 1..11: note = Synthetic
LGGPSVFLFPP

SEQ ID NO: 89 - protein, 8 aa, synthetic construct
  REGION 1..8: note = Synthetic
PAPELLGG

SEQ ID NO: 90 - protein, 13 aa, synthetic construct
  REGION 1..13: note = Synthetic
LGGPSVFLFPPKP

SEQ ID NO: 91 - protein, 9 aa, synthetic construct
  REGION 1..9: note = Synthetic
AVYYCARNV

SEQ ID NO: 92 - protein, 9 aa, synthetic construct
  REGION 1..9: note = Synthetic
FPLAPSSKS

SEQ ID NO: 93 - protein, 9 aa, synthetic construct
  REGION 1..9: note = Synthetic
LAPSSKSTS

SEQ ID NO: 94 - protein, 9 aa, synthetic construct
  REGION 1..9: note = Synthetic
APSSKSTSG

SEQ ID NO: 95 - protein, 9 aa, synthetic construct
  REGION 1..9: note = Synthetic
PSSKSTSGG

SEQ ID NO: 96 - protein, 9 aa, synthetic construct
  REGION 1..9: note = Synthetic
CEVTHQGLS

SEQ ID NO: 97 - protein, 9 aa, synthetic construct
  REGION 1..9: note = Synthetic
VDKKVEPKS

SEQ ID NO: 98 - protein, 9 aa, synthetic construct
  REGION 1..9: note = Synthetic
TKSFNRGEC

SEQ ID NO: 99 - protein, 9 aa, synthetic construct
  REGION 1..9: note = Synthetic
KKVEPKSCD

SEQ ID NO: 100 - protein, 9 aa, synthetic construct
  REGION 1..9: note = Synthetic
KVEPKSCDK

SEQ ID NO: 101 - protein, 9 aa, synthetic construct
  REGION 1..9: note = Synthetic
VEPKSCDKT

SEQ ID NO: 102 - protein, 9 aa, synthetic construct
  REGION 1..9: note = Synthetic
EPKSCDKTH

SEQ ID NO: 103 - protein, 9 aa, synthetic construct
  REGION 1..9: note = Synthetic
KSCDKTHTC

SEQ ID NO: 104 - protein, 9 aa, synthetic construct
  REGION 1..9: note = Synthetic
ELLGGPSVF

SEQ ID NO: 105 - protein, 9 aa, synthetic construct
  REGION 1..9: note = Synthetic
LLGGPSVFL

SEQ ID NO: 106 - protein, 10 aa, synthetic construct
  REGION 1..10: note = Synthetic
ELLGGPSVFL

SEQ ID NO: 107 - protein, 10 aa, synthetic construct
  REGION 1..10: note = Synthetic
PELLGGPSVF

SEQ ID NO: 108 - protein, 10 aa, synthetic construct
  REGION 1..10: note = Synthetic
LLGGPSVFLF

SEQ ID NO: 109 - protein, 9 aa, synthetic construct
  REGION 1..9: note = Synthetic
YCARYFFGS

SEQ ID NO: 110 - protein, 9 aa, synthetic construct
  REGION 1..9: note = Synthetic
CARYFFGSS

SEQ ID NO: 111 - protein, 9 aa, synthetic construct
  REGION 1..9: note = Synthetic
YFFGSSPNW

SEQ ID NO: 112 - protein, 9 aa, synthetic construct
  REGION 1..9: note = Synthetic
VYYCARYFF

SEQ ID NO: 113 - protein, 9 aa, synthetic construct
  REGION 1..9: note = Synthetic
YCARYFFGS

SEQ ID NO: 114 - protein, 9 aa, synthetic construct
  REGION 1..9: note = Synthetic
CARYFFGSS

SEQ ID NO: 115 - protein, 9 aa, synthetic construct
  REGION 1..9: note = Synthetic
YFFGSSPNW

SEQ ID NO: 116 - protein, 8 aa, synthetic construct
  REGION 1..8: note = Synthetic
PVAGPSVF

SEQ ID NO: 117 - protein, 8 aa, synthetic construct
  REGION 1..8: note = Synthetic
VAGPSVFL

SEQ ID NO: 118 - protein, 9 aa, synthetic construct
  REGION 1..9: note = Synthetic
PVAGPSVFL

SEQ ID NO: 119 - protein, 8 aa, synthetic construct
  REGION 1..8: note = Synthetic
VAGPSVFL

SEQ ID NO: 120 - protein, 8 aa, synthetic construct
  REGION 1..8: note = Synthetic
AGPSVFLF

SEQ ID NO: 121 - protein, 13 aa, synthetic construct
  REGION 1..13: note = Synthetic
SSPVTKSFNRGEC

SEQ ID NO: 122 - protein, 9 aa, synthetic construct
  REGION 1..9: note = Synthetic
PSSKSTSGG

SEQ ID NO: 123 - protein, 7 aa, synthetic construct
  REGION 1..7: note = Synthetic
SSLSASV

SEQ ID NO: 124 - protein, 9 aa, synthetic construct
  REGION 1..9: note = Synthetic
HKVYACEVT

SEQ ID NO: 125 - protein, 7 aa, synthetic construct
  REGION 1..7: note = Synthetic
WGGDGFY

SEQ ID NO: 126 - protein, 9 aa, synthetic construct
  REGION 1..9: note = Synthetic
KKVEPKSCD

SEQ ID NO: 127 - protein, 9 aa, synthetic construct
  REGION 1..9: note = Synthetic
FIFPPSDEQ

SEQ ID NO: 128 - protein, 6 aa, synthetic construct
  REGION 1..6: note = Synthetic
FNRGEC

SEQ ID NO: 129 - protein, 10 aa, synthetic construct
  REGION 1..10: note = Synthetic
YYCSRWGGDF

SEQ ID NO: 130 - protein, 13 aa, synthetic construct
  REGION 1..13: note = Synthetic
SSPVTKSFNRGEC

SEQ ID NO: 131 - protein, 8 aa, synthetic construct
  REGION 1..8: note = Synthetic
PSSKSTSG

SEQ ID NO: 132 - protein, 10 aa, synthetic construct
  REGION 1..10: note = Synthetic
YYCSRWGGDF

SEQ ID NO: 133 - protein, 4 aa, synthetic construct
  REGION 1..4: note = Synthetic
EVQL

SEQ ID NO: 134 - protein, 5 aa, synthetic construct
  REGION 1..5: note = Synthetic
EPKSC

SEQ ID NO: 135 - protein, 7 aa, synthetic construct
  REGION 1..7: note = Synthetic
GLVQPGG

SEQ ID NO: 136 - protein, 8 aa, synthetic construct
  REGION 1..8: note = Synthetic
AMDYWGQG

SEQ ID NO: 137 - protein, 8 aa, synthetic construct
  REGION 1..8: note = Synthetic
GTKVEIKR

SEQ ID NO: 138 - protein, 9 aa, synthetic construct
  REGION 1..9: note = Synthetic
VEIKRTVAA

SEQ ID NO: 139 - protein, 10 aa, synthetic construct
  REGION 1..10: note = Synthetic
GGPSVFLFPP

SEQ ID NO: 140 - protein, 8 aa, synthetic construct
  REGION 1..8: note = Synthetic
KGQPREPQ

SEQ ID NO: 141 - protein, 8 aa, synthetic construct
  REGION 1..8: note = Synthetic
VAAPSVFI

SEQ ID NO: 142 - protein, 9 aa, synthetic construct
  REGION 1..9: note = Synthetic
DYWGQGTLV

SEQ ID NO: 143 - protein, 9 aa, synthetic construct
  REGION 1..9: note = Synthetic
SVFLFPPKP

SEQ ID NO: 144 - protein, 10 aa, synthetic construct
  REGION 1..10: note = Synthetic
APIEKTISKA

SEQ ID NO: 145 - protein, 9 aa, synthetic construct
  REGION 1..9: note = Synthetic
EVTHQGLSS

SEQ ID NO: 146 - protein, 9 aa, synthetic construct
  REGION 1..9: note = Synthetic
LAPSSKSTS

SEQ ID NO: 147 - protein, 9 aa, synthetic construct
  REGION 1..9: note = Synthetic
KKVEPKSCD

SEQ ID NO: 148 - protein, 7 aa, synthetic construct
  REGION 1..7: note = Synthetic
PPTFGQG

SEQ ID NO: 149 - protein, 5 aa, synthetic construct
  REGION 1..5: note = Synthetic
NRGEC

SEQ ID NO: 150 - protein, 10 aa, synthetic construct
  REGION 1..10: note = Synthetic
KKVEPKSCDK

SEQ ID NO: 151 - protein, 8 aa, synthetic construct
  REGION 1..8: note = Synthetic
PAPELLGG

SEQ ID NO: 152 - protein, 9 aa, synthetic construct
  REGION 1..9: note = Synthetic
PAPELLGGP

SEQ ID NO: 153 - protein, 4 aa, synthetic construct
  REGION 1..4: note = Synthetic
CEGR

SEQ ID NO: 154 - protein, 5 aa, synthetic construct
  REGION 1..5: note = Synthetic
CEGRN

SEQ ID NO: 155 - protein, 7 aa, synthetic construct
  REGION 1..7: note = Synthetic
CEGRNFS

SEQ ID NO: 156 - protein, 8 aa, synthetic construct
  REGION 1..8: note = Synthetic
CEGRNFSK

SEQ ID NO: 157 - protein, 10 aa, synthetic construct
  REGION 1..10: note = Synthetic
CEGRNFSKTV

SEQ ID NO: 158 - protein, 11 aa, synthetic construct
  REGION 1..11: note = Synthetic
CEGRNFSKTVP

SEQ ID NO: 159 - protein, 12 aa, synthetic construct
  REGION 1..12: note = Synthetic
CEGRNFSKTVPS

SEQ ID NO: 160 - protein, 13 aa, synthetic construct
  REGION 1..13: note = Synthetic
CEGRNFSKTVPSS

SEQ ID NO: 161 - protein, 14 aa, synthetic construct
  REGION 1..14: note = Synthetic
CEGRNFSKTVPSSL

SEQ ID NO: 162 - protein, 16 aa, synthetic construct
  REGION 1..16: note = Synthetic
CEGRNFSKTVPSSLGQ

SEQ ID NO: 163 - protein, 17 aa, synthetic construct
  REGION 1..17: note = Synthetic
CEGRNFSKTVPSSLGQH

SEQ ID NO: 164 - protein, 18 aa, synthetic construct
  REGION 1..18: note = Synthetic
CEGRNFSKTVPSSLGQHT

SEQ ID NO: 165 - protein, 19 aa, synthetic construct
  REGION 1..19: note = Synthetic
CEGRNFSKTVPSSLGQHTV

SEQ ID NO: 166 - protein, 20 aa, synthetic construct
  REGION 1..20: note = Synthetic
CEGRNFSKTVPSSLGQHTVE

SEQ ID NO: 167 - protein, 5 aa, synthetic construct
  REGION 1..5: note = Synthetic
CDKTH

SEQ ID NO: 168 - protein, 10 aa, synthetic construct
  REGION 1..10: note = Synthetic
CDKTHTCPPC

SEQ ID NO: 169 - protein, 12 aa, synthetic construct
  REGION 1..12: note = Synthetic
CDKTHTCPPCPA

SEQ ID NO: 170 - protein, 13 aa, synthetic construct
  REGION 1..13: note = Synthetic
CDKTHTCPPCPAP

SEQ ID NO: 171 - protein, 15 aa, synthetic construct
  REGION 1..15: note = Synthetic
CDKTHTCPPCPAPEL

SEQ ID NO: 172 - protein, 18 aa, synthetic construct
  REGION 1..18: note = Synthetic
CDKTHTCPPCPAPELLGG

SEQ ID NO: 173 - protein, 5 aa, synthetic construct
  REGION 1..5: note = Synthetic
CKPEV

SEQ ID NO: 174 - protein, 4 aa, synthetic construct
  REGION 1..4: note = Synthetic
CSKP

SEQ ID NO: 175 - protein, 5 aa, synthetic construct
  REGION 1..5: note = Synthetic
CSKPE

SEQ ID NO: 176 - protein, 9 aa, synthetic construct
  REGION 1..9: note = Synthetic
CSKPEVKKD

SEQ ID NO: 177 - protein, 10 aa, synthetic construct
  REGION 1..10: note = Synthetic
CSKPEVKKDV

SEQ ID NO: 178 - protein, 11 aa, synthetic construct
  REGION 1..11: note = Synthetic
CSKPEVKKDVP

SEQ ID NO: 179 - protein, 12 aa, synthetic construct
  REGION 1..12: note = Synthetic
CSKPEVKKDVPT

SEQ ID NO: 180 - protein, 14 aa, synthetic construct
  REGION 1..14: note = Synthetic
CSKPEVKKDVPTNS

SEQ ID NO: 181 - protein, 16 aa, synthetic construct
  REGION 1..16: note = Synthetic
CSKPEVKKDVPTNSPK

SEQ ID NO: 182 - protein, 17 aa, synthetic construct
  REGION 1..17: note = Synthetic
CSKPEVKKDVPTNSPKH

SEQ ID NO: 183 - protein, 18 aa, synthetic construct
  REGION 1..18: note = Synthetic
CSKPEVKKDVPTNSPKHN

(19) United States
(12) Patent Application Publication
(10) Pub. No.: US 2023/0221333 A1
(43) Pub. Date: Jul. 13, 2023

(54) Methods for Middle Down Antibody Characterization

(71) Applicants: Genentech, Inc., South San Francisco, CA (US); Universiteit Utrecht Holding B.V., Utrecht (NL)

(72) Inventors: Wendy Noel Sandoval, Millbrae, CA (US); Tomislav Caval, Utrecht (NL); Albert J.R. Heck, Utrecht (NL); Elizabeth Sara Hecht, Oakland, CA (US)

(73) Assignees: Genentech, Inc., South San Francisco, CA (US); Universiteit Utrecht Holding B.V., Utrecht, CA (NL)

(21) Appl. No.: 18/155,299

(22) Filed: Jan. 17, 2023

Related U.S. Application Data
(63) Continuation of application No. PCT/US2021/042164, filed on Jul. 19, 2021.
(60) Provisional application No. 63/053,899, filed on Jul. 20, 2020.

(51) Int. Cl. G01N 33/68

(52) U.S. Cl. CPC G01N 33/6848; G01N 33/6824; G01N 2560/00; G01N 2333/96466

(57) ABSTRACT

This disclosure relates to new methods for antibody characterization sequencing, such as middle down antibody characterization and sequencing, for example, for de novo antibody sequencing, identifying known antibodies in a sample, or verifying the sequence of antibodies in a sample. In some embodiments, the methods involve exposing antibodies to cathepsin D, cathepsin L, and/or cathepsin D and L, followed by mass spectrometry and sequence identification and deconvolution.

### CROSS REFERENCE TO RELATED APPLICATIONS

This application claims priority to U.S. Provisional Application No. 63/053,899, filed Jul. 20, 2020, the contents of which are incorporated herein in their entirety.

### FIELD

This disclosure relates to new methods for antibody characterization sequencing, such as middle down antibody characterization and sequencing, for example, for de novo antibody sequencing, identifying known antibodies in a sample, or verifying the sequence of antibodies in a sample. In some embodiments, the methods involve exposing antibodies to cathepsin D, cathepsin L, and/or cathepsin D and L, followed by mass spectrometry and sequence identification and deconvolution.

### BACKGROUND

Approaches currently used to identify protein sequences, such as antibody sequences, in samples include bottom-up analysis, middle-down analysis, and top-down analysis. In a bottom-up analysis, which is used in the vast majority of cases, often several different proteases may be used (e.g. 4-5 proteases) to generate relatively short, overlapping peptides (e.g., 9-30 amino acids long or about 1-5 kDa) for liquid chromatography (LC) and mass spectrometry (MS) analysis. If such protocols are conducted to determine the sequence of an unknown antibody (i.e., de novo sequencing), for example, a large number of mass spectra must be generated and then assembled using specialized sequencing programs that use information on the extracted mass shifts between the product ion peaks. One limitation of this approach is that accuracy of the sequence analysis may rely on obtaining very high quality MS (e.g., tandem MS (MS/MS) fragmentation efficiency & low mass error) data and on detecting peptides that span the entirety of the protein. In addition, bottom-up analysis can create software challenges as it may require correctly piecing together sequence information from a large number of small peptides into a complete protein sequence. Middle-down approaches, which use a smaller number of peptides or protein fragments, may help to alleviate these software challenges.

Middle-down approaches can be used for antibodies, for example, where the antibodies are first cleaved near or in the hinge region between the CH1 and CH2 regions of the heavy chain, to generate F(ab′)2, F(ab′), Fc, Fd, VL-CL fragments, for example. The most commonly used enzyme is the cysteine proteinase from Streptococcus pyogenes, IdeS, which cleaves after the hinge region to create a F(ab′)2 fragment and an Fc/2 fragment. After reduction of disulfide bonds, a light chain (LC) fragment, an Fd′ fragment, and Fc fragments may result, each of which is about 25 kDa in size and about 200 amino acids in length.

These relatively long fragments, when analyzed using common MS instrumentation, are difficult to completely fragment and therefore may not be suitable for de novo sequencing. For example, currently common MS instrumentation includes time of flight with collisional induced dissociation or Orbitrap™ with higher-energy collisional dissociation (HCD) instruments that lack electron induced dissociation (a highly efficient and orthogonal fragmentation approach) and that lack ultraviolet photo dissociation (which is also suitable for very large polypeptides). Thus, middle-down approaches that provide only limited coverage may need to be supplemented with bottom-up sequencing methods for de novo antibody sequencing. Hence, there is a need for alternative middle-down methods that yield protein fragments more suited to the industry-standard LC-MS/MS CID and HCD instruments commonly in use, and that are, therefore, more practical.

### SUMMARY

This disclosure includes, inter alia, methods for cleaving an antibody, comprising mixing the antibody with cathepsin L, cathepsin D, or a combination of cathepsin L and cathepsin D to obtain one or more antibody fragments, wherein the antibody comprises a light chain comprising a light chain variable region (VL) and a light chain constant region (CL) and a heavy chain comprising a heavy chain variable region (VH) and a heavy chain constant region (CH), and wherein the cathepsin L and/or D cleaves the antibody between the VL and CL and/or between the VH and CH regions to create VL and/or VH antibody fragments and CL and/or CH antibody fragments. In some embodiments, the methods also comprise isolating one or more of the antibody fragments after the cleavage. In some embodiments, the antibody fragments are not isolated after the cleavage. In some embodiments, after the cleavage, one or more antibody fragments are analyzed by mass spectrometry.

For example, the disclosure also encompasses methods for analyzing the sequence of an antibody, comprising: (a) cleaving the antibody with cathepsin L, cathepsin D, or a combination of cathepsin L and cathepsin D to obtain one or more antibody fragments, wherein the antibody comprises a light chain comprising a light chain variable region (VL) and a light chain constant region (CL) and a heavy chain comprising a heavy chain variable region (VH) and a heavy chain constant region (CH), and wherein the cathepsin L and/or D cleaves the antibody between the VL and CL and/or between the VH and CH regions to create VL and/or VH antibody fragments and CL and/or CH antibody fragments; (b) optionally isolating one or more of the antibody fragments after the cleavage; and (c) performing mass spectrometry (MS) analysis of the one or more antibody fragments.

In any of the above methods, in some embodiments the antibody is an IgG antibody, such as a human IgG1, IgG2, IgG2A, IgG2B, or IgG4 antibody. In some embodiments, the cleavage generates VL and/or VH fragments. In some such embodiments, MS analysis is performed on the VL and/or VH fragments. In some of the methods herein, the heavy chain constant region comprises at least a CH1 region, and optionally further comprises a hinge, CH2 region, and/or CH3 region. In some methods herein, the antibody heavy chain constant region comprises at least a CH1, hinge, and CH2 region, and wherein the cathepsin L and/or cathepsin D further cleaves the antibody between the CH1 region and the hinge.

In some embodiments, the antibody is cleaved with cathepsin L. In some embodiments, the antibody is cleaved with cathepsin D. In some embodiments, the antibody is cleaved with a combination of cathepsin L and cathepsin D. In some cases, an additional enzyme, such as IdeS or another protease, is also used. In other cases, the enzyme used for cleavage consists of cathepsin L, consists of cathepsin D, or consists of the combination of cathepsin L and cathepsin D. In some cases, the method comprises incubating the antibody simultaneously with both cathepsin L and cathepsin D.

In some embodiments, cleavage is conducted so as to achieve at least 50%, at least 60%, at least 70%, at least 80%, at least 85%, at least 90%, at least 95%, at least 98%, or 100% cleavage between the VL and CL and between the VH and CH regions. In some cases, the cleavage is conducted so as to achieve at least 50%, at least 60%, at least 70%, at least 80%, at least 85%, at least 90%, at least 95%, at least 98%, or 100% cleavage at or below the hinge. In some cases, the antibody is an IgG antibody and is cleaved with a combination of cathepsin L and cathepsin D, wherein the cleavage results in VL, VH, CL, CH1, CH2, CH3, CH2-CH3, CL+CH1 (bonded), F(ab′), and F(ab′)2 fragments. In some embodiments, the cleavage results in fragments comprising each of a light chain CDR1, CDR2, and CDR3, such as a VL, F(ab′), or F(ab′)2 fragment, and/or fragments comprising each of a heavy chain CDR1, CDR2, and CDR3, such as a VH, F(ab′), or F(ab′)2 fragment.

In some methods herein, cleavage is conducted by incubating the antibody with the cathepsin L, cathepsin D, or combination of cathepsin D and L at pH 2-8 (such as pH 2-7, pH 2-6, pH 3-6, pH 3-5, pH 2, pH 2.5, pH 3, pH 3.5, pH 4, pH 4.5, pH 5, pH 5.5, pH 6, pH 7, or pH 8), at a temperature from room temperature to 50° C., and in the presence of no more than 50% organic solvent (e.g. acetonitrile, methanol, ethanol, or isopropyl alcohol), wherein the antibody is in a native state. In some methods herein, the cleavage is conducted in the presence of one or more organic solvents (e.g. methanol, ethanol, isopropyl alcohol, or acetonitrile) at a concentration of 0-50%, 5-50%, 5-30%, 0-30%, 10-30%, 0-10%, 5-15%, 10-20%, 15-25%, or 20-30%. In some methods herein, including those comprising one or more organic solvents (e.g. methanol, ethanol, isopropyl alcohol, or acetonitrile) at a concentration of 0-50%, 5-50%, 5-30%, 0-30%, 10-30%, 0-10%, 5-15%, 10-20%, 15-25%, or 20-30%, the pH is from 3 to 5, such as 3, 3.5, 4, 4.5, or 5. In some such cases, the pH is from 3.5 to 4.5. In some such cases, the pH is 4. In some cleavage reactions, no more than 30% organic solvent is present. In some such cases, cleavage is conducted in the presence of 10-30% organic solvent (e.g. 10-30% acetonitrile, 10-30% methanol, 10-30% ethanol, or 10-30% isopropyl alcohol). In some cleavage reactions, no more than 10% organic solvent is present. In some cleavage reactions, the temperature is between 37° C. and 50° C.

The disclosure herein also includes methods for analyzing the sequence of an antibody, comprising: (a) leaving the antibody with a combination of cathepsin L and cathepsin D to obtain one or more antibody fragments that comprise at least a light chain variable region (VL) fragment and/or a heavy chain variable region (VH) fragment, (i) wherein the antibody is in a native state; (ii) wherein the antibody comprises a light chain comprising a light chain variable region (VL) and a light chain constant region (CL) and a heavy chain comprising a heavy chain variable region (VH) and a heavy chain constant region (CH), and wherein the cathepsin L and D cleave the antibody between the VL and CL and/or between the VH and CH regions to create VL and/or VH antibody fragments and CL and/or CH antibody fragments, and (iii) wherein the cleavage is performed at a temperature between 25° C. and 50° C. and a pH from 3 to 5, and in the presence of no more than 30% organic solvent; (b) optionally isolating one or more of the antibody fragments after the cleavage; and (c) performing mass spectrometry (MS) analysis of the one or more antibody fragments.

In any of the methods herein, following cleavage, the one or more antibody fragments may be subjected to one or more of buffer exchange, chromatography (e.g. liquid chromatography such as high performance liquid chromatography, or capillary electrophoresis), filtration (e.g. molecular weight cut-off filtration), reduction of disulfide bonds, exposure to guanidine hydrochloride, or alkylation. For example, one or more of these steps may be performed prior to an MS analysis or on a portion of the sample undergoing MS analysis (e.g., reduction or alkylation of a part of the sample so as to compare MS assignments with and without those alterations). In some embodiments, one or more antibody fragments following cleavage are isolated by chromatography or filtration. In some such cases, one or more antibody fragments are isolated by liquid chromatography.

In some embodiments, where MS analysis is performed, the MS comprises LC-MS or LC-MS/MS. In other cases, the one or more antibody fragments are not isolated following cleavage. In some embodiments, where mass spectrometry is performed, the mass spectrometry comprises direct infusion mass spectrometry (DIMS), static spray infusion mass spectrometry, or flow injection mass spectrometry.

In some embodiments herein, where mass spectrometry is performed, the mass spectrometry data is used to determine the amino acid sequence of at least a 10 amino acid stretch of one antibody fragment. In some embodiments, the amino acid sequence of at least a 15 amino acid stretch of one antibody fragment is determined. In some embodiments, the amino acid sequence of at least a 20 amino acid stretch of one antibody fragment is determined. In some embodiments, the amino acid sequence of at least one antibody CDR region is determined. In some embodiments, the sequence of the VH and/or VL CDR1, CDR2, and CDR3 is determined. In some embodiments, the complete amino acid sequence of at least one antibody fragment, such as a VH and/or VL fragment, is determined. In some embodiments, the sequence is determined by top-down analysis. In some embodiments, the sequence is further analyzed or is confirmed by bottom-up analysis. In some embodiments, the amino acid sequence of the antibody VH and/or VL regions is unknown. In some embodiments, the amino acid sequence of the antibody is unknown. Some embodiments also comprise performing MS on the CL and/or CH regions of the antibody, such as on a CL, CH1, CH2, and/or CH3 fragment generated from the cleavage. In some cases, any of the methods herein further comprises performing Edman degradation on at least one antibody fragment. In some methods herein, the cleavage is conducted at a cathepsin L and/or cathepsin D to antibody ratio of: 1:20 to 1:2000, 1:20 to 1:500, 1:50 to 1:500, 1:100 to 1:500, 1:200 to 1:1000, 1:200 to 1:2000, 1:500 to 1:2000, 1:1000 to 1:2000, or 1:20, 1:50, 1:100, 1:200, 1:300, 1:400, 1:500, or 1:1000. In some methods herein, the antibody remains in the native state after the cleavage. In some methods herein, the antibody is not treated with denaturing agents or agents that reduce disulfide bonds during or after the cleavage. In some methods herein, the antibody retains its disulfide bonding during and after the cleavage. Some methods herein comprise performing mass spectrometry (MS) analysis of one or more antibody fragments following the cleavage, wherein the antibody remains in the native state and is not treated with denaturing agents or agents that reduce disulfide bonds prior to the MS analysis.

The disclosure also includes compositions comprising antibody fragments produced according to the methods above. In some cases, compositions comprise IgG antibody fragments produced from cleavage with cathepsin L, cathepsin D, or a combination of cathepsin L and D, wherein the antibody fragments comprise one or both of VH and VL fragments, and at least one, at least two, or at least three of the following fragments: CL, CH1, CH2, CH3, CH2-CH3, CL+CH1 (bonded), F(ab′), and F(ab′)2. In some cases, the VH and/or VL fragments comprise from 90 to 150 amino acids in length, such as from 95 to 140 amino acids, such as 100 to 140 amino acids, or such as from 100 to 120 amino acids. In some such cases, the VH and/or VL fragments have a molecular mass of 10-16 kDa, such as 10-13 kDa, such as 10-12 kDa, such as 10-11 kDa, or such as 11-12 kDa.

The present disclosure also comprises kits for use in digesting a protein with cathepsin L, cathepsin D, or a combination of cathepsin L and cathepsin D, the kits comprising (a) cathepsin L and/or cathepsin D; (b) one or more reaction buffers; and optionally (c) instructions for use in digesting proteins. In some cases, a kit provides reagents for use in cleaving an antibody according to the methods above. In some kits, the reaction buffer is at pH 2-8, pH 2-7, pH 2-6, pH 2-5, pH 3-6, pH 3-5, pH 3-4, pH 4-5, pH 2, pH 2.5, pH 3, pH 3.5, pH 4, pH 4.5, pH 5, pH 5.5, pH 6, pH 7, or pH 8; and the reaction buffer comprises one or more organic solvents. In some embodiments, the reaction buffer comprises one or more organic solvents (e.g. methanol, ethanol, isopropyl alcohol, or acetonitrile) at a concentration of 0-50%, 5-50%, 5-30%, 0-30%, 10-30%, 0-10%, 5-15%, 10-20%, 15-25%, or 20-30%. In some cases, the reaction buffer is at pH 3, 3.5, 4, 4.5, or 5. In some kits, the reaction buffer is at pH 4.

It is to be understood that both the foregoing general description and the following detailed description are exemplary and explanatory only and are not restrictive of the claims. The accompanying drawings, which are incorporated in and constitute a part of this specification, illustrate certain embodiments or aspects, and together with the description, serve to explain the principles described herein.

### BRIEF DESCRIPTION OF THE DRAWINGS

FIGS. 1A-D show spectra for rituximab exposed to Cathepsin L. FIGS. 1A-B show the full spectrum rituximab exposed to Cathepsin L, showing CDR L3 clips at masses around 10 kDa, “one-arm” clips around 47 kDa, complements of one-arm clips around 100 kDa (for example, 100,238 is the full monoclonal antibody (mAb) with G1F/G1F at 147,400 missing 47,178, along with 18 for water), and intact mAb around 147 kDa. FIGS. 1C-D show charge deconvolution of the same rituximab exposed to Cathepsin L spectrum, focusing on the m/z range (3000-4000) and m range (45000-49000) of the one-arm clips.

FIGS. 2A-D show spectra for rituximab exposed to Cathepsin D. FIGS. 2A-B shows one-arm clips. FIGS. 2C-D show F(ab′)2 clips.

FIGS. 3A-D show spectra of obinutuzumab exposed to Cathepsin L. FIGS. 3A-B show full mAb peaks. 148630.4 Da is a good match for the mAb with G0/G0 glycosylation, 148,834.1 for an extra GlcNAc, 149038.0 with two extra GlcNAcs. Obinutuzumab is glyco-engineered to have mostly afucosylated glycans. FIGS. 3C-D show one-arm clips.

FIGS. 4A-H show spectra for obinutuzumab exposed to Cathepsin D. FIGS. 4A-B show the full spectrum. FIGS. 4C-D show full mAb peaks. 148628.3 Da is a good match for the mAb with G0/G0 glycosylation, 148,832.9 for an extra GlcNAc, 149034.7 with two extra GlcNAcs. FIGS. 4E-F show full one-arm clips. FIGS. 4G-H show F(ab′)2 clips.

FIGS. 5A-D show spectra for eculizumab exposed to Cathepsin L. FIGS. 5A-B show the full spectrum. FIGS. 5C-D show CDR H3 clips.

FIGS. 6A-D show spectra for eculizumab exposed to Cathepsin D. FIGS. 6A-B show the full spectrum. FIGS. 6C-DB show F(ab′)2 clips.

FIGS. 7A-B show the native mass spectrum of rituximab digested by Cathepsin D. FIG. 7A shows deconvolution of the full m/z range gives peptides from 9-98 kDa and shows a significant amount of intact Ab (˜147 kDa). The peak at 97,684 is generated from cleavage below the hinge at LLGGPSVF.L. Asymmetric clips, such as 97,538 formed by F(ab′) LLGGPSVF.L+F(ab′) LLGGPSV.F were also observed and are shown in the inset. FIG. 7B shows deconvolution over the 47 kDa region reveals the multiplicity in clips observed at a specific site location, the F(ab′), which is shown on the IgG1 crystal structure (PDB1HZH).

FIG. 8 shows the preferred cleavage sites of Cathepsin L (grey lines) and D (plain black lines) alone and in combination on human IgG1, IgG2-B, and IgG1 antibodies. For the IgG1 class, Cathepsins L and D each produced cleavages at the heavy chain (HC) and light chain (LC) CDR3, above the hinge (F(ab′)), and throughout the heavy chain hinge region (dashed grey and black lines). Cathepsin D alone uniquely cut the sequence PSVFL.F to yield the F(ab′)2. (Solid black line.) A combination of Cathepsins L and D produced further cleavages at the locations shown by black lines with diamonds at each end. No cleavages were observed in eculizumab (IgG2-B) within the hinge due to its different disulfide pattern. In the IgG1 bispecific, no cleavages were observed in the Fc anti-CD3 arm (hole), compared to those observed in the anti-Her2 (knob) arm.

FIGS. 9A-B show comparison of the digestion efficiency of Cathepsin L and D across different treatments. FIG. 9A shows all identified polypeptides, as reported in Tables 3 and 11. The polypeptides were summed and taken against the intensity of intact Trastuzumab. FIG. 9B shows UV peak areas for all peptide peaks (corresponding to the EIC elution time) taken as a ratio to the main Ab peak. Error bars represent the standard deviation of the measurement.

FIGS. 10A-E show examples of UV and TIC measurements of the one-pot Cathepsin L and D digests. The insets show selected MS spectra averaged across their elution time window and deconvolved in Intact Mass (Protein Metrics). The masses in these figures may vary slightly from the masses given in the tables disclosed herein because the exact masses depend upon the time, mass, and m/z ranges, as well as other parameters, used in charge deconvolution.

FIGS. 11A-D show spectra for static infusion of the cathepsin pH 4 sample. The sample was buffer exchanged into 50 mM ammonium acetate using a Biorad Microspin® column, on an ultra-high mass range (UHMR) mass spectrometer. MS1 spectra was obtained at 17,500 resolving power at 200 m/z and charge states were confirmed in SIM mode at 50K or 100K resolving power. FIG. 11A shows Rf settings were tuned and optimized for high masses (>4000 m/z). FIG. 11B shows Rf settings were tuned and optimized for mid-range masses (1500-4000 m/z). FIGS. 11C and 11D show high resolution mass spectra of the 12.1 kDa species and the 47 kDa species, respectively.

FIG. 12 shows Edman degradation assignments of the Cathepsin L and Cathepsin D optimized digests. Amino acids in “( )” are less reliable than those identified with good confidence.

FIGS. 13A-C show MS2 spectra of selected Cathepsin digest products. FIG. 13A shows mass spectrum of the 12.1 kDa species. FIG. 13B shows mass spectrum of the 47 kDa species. FIG. 13C shows mass spectrum of the 98 kDa species.

FIGS. 14A-B show top down annotation of selected polypeptides. Coverage of the (FIG. 14A) 12,121.5 Da and (FIG. 14B) 98 kDa products is shown, where y ions in light grey correspond to the HC sequence ending in G, in dark grey with GG, and with a triangle to a y-ion plus unspecified covalent cross-linked modification. Black represents HC b ions or LC b/y ions. The spectra were collected by nESI infusion, deconvoluted using the Xtract algorithm, and matched to fragments with a tolerance of 10 ppm in ProSight Lite and using in house programs.

FIGS. 15A-C show amino acid enrichment motifs. FIG. 15A shows Cathepsin L. FIG. 15B shows Cathepsin D. FIG. 15C shows one-pot Cathepsin L and Cathepsin D digests. The amino acid preferences for p4=p4′ were evaluated in Seq2Logo 2.0 [71].

FIG. 16 shows the molecular model of trastuzumab and cleavage sites observed following the optimized digestion protocol. The trastuzumab (PDB 6BI2) LC is shown on the right/top side (bottom view orientation) and the HC AA 1-221 is shown on the left/top side (bottom view orientation). The HC CH2 and CH3 regions are shown in black and combined, the structure shown is a single Ab arm (half an antibody). The trastuzumab F(Ab′) was aligned to residues 1-214 of a full-length IgG1 crystal structure (PDB 1HZH) using the in-house program GYST and modeled in PyMol 2.3.5. The CH1, CH2, and hinge region of the aligned IgG1 (AA 228-478) has 90.9% identity to trastuzumab, where all cleavage sites fell over a region of identical homology. The disulfide bonds are not shown and the Fc glycans are shown as sticks in white. Any cleavage sites are shown as sphere models and are colored black.

### FURTHER DESCRIPTION OF CERTAIN EMBODIMENTS

### Definitions

Unless otherwise defined, scientific and technical terms used in connection with the present invention shall have the meanings that are commonly understood by those of ordinary skill in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

A “sample” as used herein refers to any specimen that may contain a protein or antibody needing analysis.

The terms “polypeptide” and “protein” are used interchangeably and refer to a polymer of amino acid residues. Such polymers of amino acid residues may contain natural and/or non-natural amino acid residues, and include, but are not limited to, peptides, oligopeptides, dimers, trimers, and multimers of amino acid residues. The terms also include polymers of amino acids that have modifications such as, for example, glycosylation, sialylation, and the like, or that are complexed with other molecules.

A “peptide” herein is a relatively short polymer of amino acids, such as on the order of 4 to 50 amino acids.

The term “antibody” or “Ab” herein is used in the broadest sense and encompasses various antibody structures, including but not limited to monoclonal antibodies (“mAb”), polyclonal antibodies, and multispecific antibodies (e.g., bispecific antibodies), so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody also includes, but is not limited to, chimeric antibodies, humanized antibodies, human antibodies, and antibodies of various species such as mouse, cynomolgus monkey, etc. The term also encompasses antigen binding fragments. The term “antigen binding fragment” includes, but is not limited to, fragments of antibodies that are capable of binding antigen, such as Fv, single-chain Fv (scFv), Fab, Fab′, and (Fab′)₂. In contrast, a “full length antibody” refers to an antibody molecule comprising all of its normal variable and constant region portions. Like other proteins and peptides, in some embodiments, antibodies may contain various types of post-translational modifications, such as glycosylations.

A “fragment” of a protein, polypeptide, or antibody (an “antibody fragment”) generally refers to a portion or region of a larger molecule, such as an Fc or an F(ab′)2 fragment of an antibody or a peptide cleaved by an enzyme from a protein. A protein fragment, such as an antibody fragment, for example, may be generated by enzymatic cleavage in the methods described in this disclosure. Or it may be generated during a mass spectrometry process.

A protein or antibody in the “native state” herein means one that retains its native folded structure and disulfide bonding and that has not been denatured, unfolded, or reduced to remove disulfide bonds. A protein in the native state, for example, may be in the presence of sufficient organic solvent to relax the native fold without denaturing or unfolding the protein.

The term “heavy chain” or “HC” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a full length heavy chain variable region and a full length heavy chain constant region, with or without a leader sequence, and with or without a C-terminal lysine (K). The term “mature full-length heavy chain” refers to a polypeptide comprising a heavy chain variable region and a heavy chain constant region, without a leader sequence, and with or without a C-terminal lysine (K). In some embodiments, a heavy chain comprises post-translational modifications such as pyro-glutamic acid modifications, while in other cases, a heavy chain does not contain such modifications.

The term “heavy chain variable region” or “VH” refers to a region comprising a heavy chain complementary determining region (CDR) 1, framework region (FR) 2, CDR2, FR3, and CDR3 of the heavy chain. In some cases, the VH also comprises some or all of FR1, prior to CDR1, and/or some or all of FR4, following CDR3. A “full length VH” is a VH that comprises a complete FR1 and a complete FR4, which precede and follow the CDR1, FR, CDR2, FR3, CDR3 segment, respectively. As used herein, a first protein segment that is “prior to” or “precedes” a second segment means that the first segment is N-terminal to the second segment. A first segment that “follows” a second segment is C-terminal to the second segment. The VH of a human IgG antibody, in EU numbering, typically comprises amino acids 1-117 of the heavy chain.

The term “light chain” or “LC” refers to a polypeptide comprising at least a light chain variable region, with or without a leader sequence. In some embodiments, a light chain comprises at least a portion of a light chain constant region. The term “full-length light chain” refers to a polypeptide comprising a full length light chain variable region and a full length light chain constant region, with or without a leader sequence. The term “mature full-length light chain” refers to a polypeptide comprising a light chain variable region and a light chain constant region, without a leader sequence. In some cases a light chain may contain post-translational modifications while in other cases it does not.

The term “light chain variable region” or “VL” refers to a region comprising a light chain CDR1, FR2, HVR2, FR3, and HVR3. In some embodiments, a light chain variable region also comprises at least a portion of an FR1 and/or an FR4. A “full length VL” is a VL that comprises a complete FR1 and a complete FR4, which precede and follow the CDR1, FR, CDR2, FR3, CDR3 segment, respectively. The VL of a human antibody, in EU numbering, typically comprises amino acids 1-107 of the heavy chain.

The term “heavy chain constant region” or “CH” as used herein refers to a region following the heavy chain variable region and encompassing one, two, or three heavy chain constant regions, C_{H}1, C_{H}2, and C_{H}3 and any segments joining those regions. The constant region of the heavy chain of an IgG antibody typically has three regions, termed CH1, CH2, and CH3, with a short “hinge” region in between CH1 and CH2. The CH2 and CH3 regions collectively form an “Fc” fragment of an antibody. The “hinge” region of an IgG class antibody refers to a short amino acid sequence region between the CH1 and CH2 portions of the heavy chain that is relatively flexible in the antibody native state. In EU numbering, the hinge region of a human IgG antibody is found at about amino acids 216-230 of the heavy chain, while the CH1 region is at about amino acids 118-215, the CH2 is at about amino acids 231-340, and the CH3 is at about amino acids 341-447.

Nonlimiting exemplary heavy chain constant regions include γ, δ, and α. Nonlimiting exemplary heavy chain constant regions also include ε and μ. In addition, an antibody comprising a γ constant region is an IgG antibody, an antibody comprising a δ constant region is an IgD antibody, and an antibody comprising an a constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

In some embodiments, a heavy chain constant region, framework region, or light chain constant region comprises one or more mutations (or substitutions), additions, or deletions that confer a desired characteristic on the antibody. For example, a nonlimiting exemplary mutation is the S241P mutation in the human IgG4 hinge region (between constant domains C_{H}1 and C_{H}2), which alters the IgG4 motif CPSCP to CPPCP, which is similar to the corresponding motif in IgG1. That mutation can result in a more stable IgG4 antibody. See, e.g., Angal et al., Mol. Immunol. 30: 105-108 (1993); Bloom et al., Prot. Sci. 6: 407-415 (1997); Schuurman et al., Mol. Immunol. 38: 1-8 (2001).

The term “light chain constant region” or “CL” as used herein refers to a region that follows the VL region, and may be termed “CL” for short. Nonlimiting exemplary light chain constant regions include λ and κ. The CL of a human antibody, in EU numbering, typically comprises amino acids 108-214 of the heavy chain.

An “IgG” or “immunoglobulin G” antibody is one of several mammalian antibody classes (others being, for example, IgA, IgM, etc.) and comprises a y constant region. It is a tetrameric protein formed from two heavy chains and two light chains. The light chain typically comprises a variable region and a light chain constant region (VL and CL, respectively). The heavy chain typically comprises a heavy chain variable region (VL), followed by the CH1, hinge, CH2, and CH3 constant regions.

As used herein, a protease such as cathepsin D or L may cleave an antibody “between” two regions, such as a VL and CL or a VH and CH (i.e. CH1). In such cases, for instance for cleavage between VL and CL or between VH and CH, this means that cleavage occurs just prior to the CDR3, within the CDR3, or within the FR4 of the VL or VH but prior to the 20^{th} amino acid in the CL or CH (i.e. CH1) segment of the antibody. This is illustrated, for instance, in the working examples herein. Cleavage occurring between a CH1 and a CH2 region, for example, may similarly occur prior to, within or following the hinge region so as to result in one fragment containing most of the CH1 region and another fragment containing most of the CH2 region.

As used herein, a protease cleavage site “preceding,” “prior to,” “before,” or “above” a certain amino acid region or position means that the cleavage occurs N-terminal to that region or position. Cleavage occurring “following” or “after” or “below” a particular position or region means cleavage C-terminal to that position or region. Similarly, a region or amino acid position that “precedes” or that is “prior to,” “before,” or “above” another amino acid region or position is N-terminal to that other amino acid region or position. A region or amino acid position that “follows” or is “after” or “below” another region or position is C-terminal to that other region or position.

“Cathepsin L” comprises a lysosomal protease expressed in eukaryotic cells and may be natural or recombinantly produced. The cathepsin L may be derived from a variety of eukaryotic organisms, such as humans or other mammals. The cathepsin L may also include genetically engineered variants of native cathepsin L that retain cathepsin L activity but that, for example, may improve activity, yield, shelf-life, or stability. Cathepsin L is also known as cathepsin L1. In some embodiments, the cathepsin L is human cathepsin L, such as recombinant human cathepsin L. Mammalian cathepsin L is expressed from the CTSL1 gene. An exemplary human cathepsin L comprises the sequence of SEQ ID NO: 10, and can also be purchased from Sigma (cat. No. C6854).

“Cathepsin D” comprises a lysosomal protease expressed in eukaryotic cells and may be natural or recombinantly produced. The cathepsin D may be derived from a variety of eukaryotic organisms, such as humans or other mammals. The cathepsin D may also include genetically engineered variants of native cathepsin D that retain cathepsin D activity but that, for example, may improve activity, yield, shelf-life, or stability. In some embodiments, the cathepsin D is human cathepsin D, such as recombinant human cathepsin D. Mammalian cathepsin D is expressed from the CTSD gene. An exemplary human cathepsin L comprises the sequence of SEQ ID NO: 9, and can also be purchased from Sigma (cat. No. C8696).

“De novo” sequencing of a protein or protein fragment refers to determining the sequence of that protein or fragment, such as a full length antibody molecule or an antigen binding fragment or other antibody fragment, wherein the sequence is not known beforehand through other means. In other words, the sequence is determined “from scratch,” without relying on any previously known sequence information.

“Bottom-up” protein sequencing methods refer to methods in which a protein is digested to form peptides, typically short in length or size (e.g. about 3-5 kDa or about 5-30 amino acids in length), for example, with an enzyme such as trypsin, and the peptides are analyzed by mass spectrometry, and then assembled via software, such as by analysis of overlapping peptides and comparison to known protein sequences.

“Top-down” sequencing involves measuring the mass of an intact protein or polypeptide and then fragmenting the whole protein via mass spectrometry into a series of product ions from which sequence information can be derived.

“Middle-down” sequencing involves breaking a protein into larger sized fragments (e.g. about 5-25 kDa), which may then be separated and further analyzed by top-down and/or bottom-up approaches. This approach typically uses proteases to generate the fragments.

As used herein, “isolating” one or more protein fragments following an enzymatic cleavage reaction refers to separating at least partially desired protein fragments from other fragments and/or separating at least partially fragments from the enzymes so that sequence or structural analysis may be performed on the fragments without interference from contaminating proteins. In some cases, a desired fragment can be isolated during mass spectrometry analysis, while in other cases it may be isolated at least in part via chromatography, filtration, or other methods.

“Liquid chromatography” or “LC” refers to a process of separating components of a sample by means of their respective interactions with a stationary phase (e.g., a column of particulate material) and a mobile (i.e., fluid) phase. LC may be performed in a single dimension (1D-LC), meaning that one separation process is run, or it may be performed in two dimensions (2D-LC), meaning that the eluate of the first separation or a portion thereof is further separated in a second separation step using a different means of separation, such as using a different mobile phase. LC encompasses, for example, HPLC and reverse phase-HPLC methods. “High-performance liquid chromatography” or “HPLC” refers to a type of LC system in which mobile phase is caused to flow through a stationary phase, such as a column, under pressure. An HPLC system may be linked to a detector such as a mass spectrometer. An HPLC process can be performed at “normal phase” (“NP” or “NP-HPLC”) or “reverse phase” (“RP” or “RP-HPLC” or “RPLC”). In an RPLC process, the stationary phase (e.g., column) is nonpolar while the mobile phase is polar, such as a water/polar organic solvent mixture or gradient. In normal phase HPLC, the stationary phase (e.g., column) is polar and the mobile phase is nonpolar. LC methods also include size exclusion chromatography (SEC), to separate polypeptides by size, and methods using phases that separate by charge or isoelectric point (pI) such as hydrophobic interaction chromatography (HIC), strong cation exchange (SCX), strong anion exchange (SAX), WSX, and other charge-variant interaction phases.

“Mass spectrometry” or “MS” refers to a technique that measures the mass to charge ratio (m/z) of one or more molecules in a sample. As used herein, “tandem MS” or “MS/MS” refers to the process by which a single ion, multiple ions, or the entire mass envelope (the precursor(s)) are moved to a fragmentation chamber and the fragmented products are then sent to a mass analyzer. Depending on the design of the mass spectrometer, the fragmentation event can happen before a single mass analyzer, between two or multiple different analyzers, or within a single mass analyzer.

MS analysis may have a variety of options. In some embodiments, the MS instrument does not comprise a quadrupole. In some embodiments, the MS instrument comprises at least one quadrupole. In some embodiments, the MS instrument comprises at least 2 quadrupole analyzers. In some embodiments, the MS instrument comprises at least 3 quadrupole analyzers. In some MS's, the detector is an ion trap, quadrupole, orbitrap, or TOF. In some embodiments, the MS instrument or method is multiple reaction monitoring (MRM), single ion monitoring (SIM), triple stage quadrupole (TSQ), quadrupole/time of flight (QTOF), quadrupole linear ion trap (QTRAP), hybrid ion trap/FTMS, time of flight/time of flight (TOF/TOF), Orbitrap instruments, ion trap instruments, parallel reaction monitoring (PRM), data dependent acquisition (DDA), data independent acquisition (DIA), multi-stage fragmentation or tandem in time MS/MS.

### Antibody Cleavage

In some embodiments herein, an antibody is cleaved with cathepsin L, cathepsin D, or a combination of cathepsin L or D. In some embodiments, the antibody is digested with cathepsin L only and no other enzymes. In some embodiments, the antibody is digested with cathepsin D only and no other enzymes. In some embodiments, the antibody is digested with both cathepsin L and cathepsin D but no other enzymes. In some embodiments, at least one other protease is also used to digest the antibody, such as IdeS. When both cathepsin L and D enzymes are used, the enzymes may be incubated with the antibody either simultaneously (i.e., in a one pot reaction) or sequentially one after the other.

In some embodiments, the antibody is an IgG antibody. In some embodiments, the antibody is a human IgG antibody, such as an IgG1, IgG2, IgG3, or IgG4 antibody. In some embodiments, the antibody is a full length IgG antibody. In some embodiments, the antibody is a full length human IgG1, IgG2, IgG3, or IgG4 antibody. In some embodiments, the antibody has a full length light chain and/or a full length heavy chain. In other embodiments, the antibody does not have a full length light chain and/or does not have a full length heavy chain. In some such cases, the antibody comprises a light chain comprising a VL and at least part of a CL region. In some such cases, the antibody comprises a VH and at least part of a CH1 region, such as, for example, a CH1, hinge, and at least part of a CH2 segment, or a CH1 and hinge only, or a CH1 only, or the N-terminal portion of a CH1 only. In some embodiments, the antibody is an antigen binding fragment such as an F(ab′)2 fragment or a Fab fragment, for example.

In some embodiments, the cleavage reaction is performed on the antibody in the native state, i.e., wherein the native antibody folding pattern is maintained and any disulfide linkages are intact and not reduced. Hence, in some embodiments, the antibody has not been subjected to reduction or denaturation. In some embodiments, however, up to 50% organic solvent may be added to relax the antibody state to some extent for optimal cleavage. For example, in some embodiments, 0-30% organic solvent may be added, such as 5-30%, 10-30%, 0-10%, 5-15%, 10-20%, 15-25%, or 20-30%. The organic solvent may comprise, for example, acetonitrile, methanol, ethanol, or isopropyl alcohol.

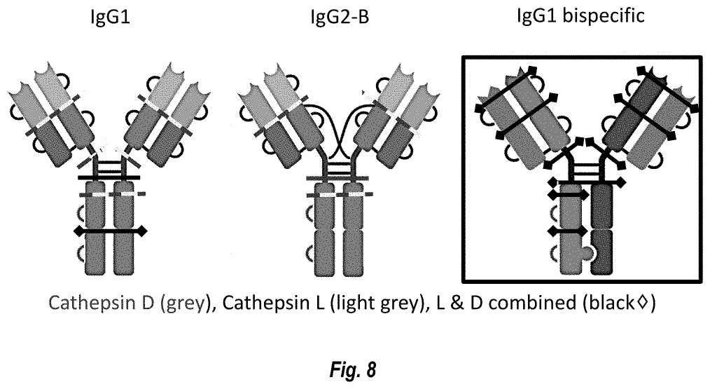

The cleavage sites for the antibodies on human IgG1 and IgG2B antibodies are shown schematically in FIG. 8. In methods herein, both cathepsin L and D may cleave the antibody at one or more locations including between the VH and CH regions as well as between the VL and CL regions, so as to clip off VH and VL antibody fragments from the antibody, which comprise at least the majority of a VH region, and the majority of a VL region, respectively. This cleavage also leaves CL and CH fragments comprising the CL and CH regions C-terminal to the cleavage site. In some embodiments, wherein the antibody is a human IgG1 for example, both cathepsin L and D also cleave above the hinge region of the antibody, yielding two F(ab′) fragments (each comprising VL plus CL and VH plus CH1) and a further fragment from the heavy chain comprising the hinge region and additional constant region sequence segments from the CH2 and CH3 regions. If cleavage takes place at both of these locations, between the variable and constant regions and above the hinge, a set of fragments comprising VL, CL, VH, CH1, and the remaining heavy chain hinge and constant region sequences, as well as Fab fragments if the cleavage is incomplete. Again, depending on the exact location of the cleavages, a CH1 fragment may comprise amino acid sequence from the C-terminal end of the VH region, and may not extend all the way to the C-terminal end of the CH1 as defined by EU numbering. Thus, the fragment may comprise most of the CH1 region. In other embodiments, wherein the antibody is a human IgG2B or IgG4, for example, cathepsin L or D might not cleave above the hinge due to different disulfide bond architecture in the antibody. (See FIG. 8.) Both cathepsin L and D may further cleave IgG1 antibodies between the CH2 and CH3 regions of the Fc portion, as shown in FIG. 8, leaving a CH2 fragment and a CH3 fragment on either side of the cleavage location. In methods herein, cathepsin D may also cleave immediately below the hinge region, thus creating a F′(ab)2 fragment and producing an Fc fragment with all or most of the Fc region sequence below the cleavage site. (See FIG. 8.) When both enzymes are used to cleave a human IgG1 antibody, for example, a series of fragments corresponding to VL, VH, CL, CH1, CH2, CH3, CL+CH1 (disulfide bonded), and F(ab′)2 may result. In some embodiments, cathepsin D and cathepsin L in combination yield VL, VH, CL, CH1, CH2, CH3, CL+CH1 (bonded), and F(ab′) fragments from a human IgG1 antibody. In some embodiments, cleavage with cathepsin L or D or both cathepsin D and L leaves VH and VL fragments of about 8-16 kDa, such as of about 10-16 kDa, 10-13 kDa, 10-12 kDa, 10-11 kDa, or 11-12 kDa. In some cases, the VH and VL fragments have on the order of 90-120 amino acids in length, such as about 95-110 amino acids in length. In some cases, simultaneous or missed cleavages will result in fragments that include various combinations of VL, VH, CL, CH1, CH2, CH3, CL+CH1, and F(ab′)2 fragments.

The Examples herein provide data showing cleavage of known antibodies by cathepsin L and/or D. As shown in Table 5a, Obinutuzumab was cleaved with cathepsin L in the VH CDR3 and at the end of the variable part of the heavy chain. And the enzyme cleaved at several locations in the heavy chain hinge region. This resulted in VH fragments of amino acids 1-99, and 1-135, 1-136, 1-137, 1-138, and 1-139 with masses between 10 and 16 kDa, for example, as well as longer light chain and heavy chain fragments such as LC fragments of amino acids 1-206 and 1-218 and HC fragments of amino acids 1-220 and LC+HC fragments with masses of 20-25 kDa and 45-50 kDa, respectively. (See Table 5a.) Exposure to cathepsin D cuts Obinutuzumab in the heavy chain hinge region, as shown in Table 5b below. For example, this cleavage leads to LC+HC fragments of about 220 to 245 amino acids in length and F(ab′)2 fragments. Tables 7, 8, and 10 show locations of cleavage for trastuzumab, which like Obinutuzumab, is a human IgG1 antibody. Trastuzumab cleaved with cathepsin L produced heavy chain fragments of amino acids 1-140 and 1-102 (i.e. VH fragments) and light chain fragments of amino acids 1-213 and 1-214 and 9-196, as well as heavy chain fragments of amino acids 100-223 (comprising the CH1 region) and light chain fragments of amino acid residues 117-214 of the CL region. (Table 8.) Cleavage with cathepsin D resulted in similar sets of fragments, light chain fragments of residues 1-214, 1-213, and heavy chain fragments of 1-140 and 1-102, as well as heavy chain fragments comprising residues 2-222 and 11-110. (Table 9.) Cleavage with both enzymes resulted in several VL fragments comprising residues 1-107, 1-111, and 1-116, longer LC fragments of residues 1-202, and VH fragments comprising positions 1-115, as well as HC constant region fragments of amino acids 241-349 and 243-341. Fragments comprising the LC and heavy chain CH1 regions combined were also produced, as well as F(ab′) and F(ab′)2 fragments. (Table 10.) Eculizumab, a human IgG2B antibody, exposed to cathepsin L, as with Obinutuzumab, results in VH fragments ending in the heavy chain CDR3, and of amino acids 1-102 to 1-106 of the heavy chain sequence. Table 6 shows that cathepsin D cuts eculizumab also in the VH CDR3 and cuts F(ab′)2 from Fc, below the hinge region.

Thus, in some embodiments, when acting on human IgG1 antibodies, cathepsins L or/and D will cleave between the VH and CH and/or between the VL and CL, specifically within or just after the HC and LC CDR3, producing VH and VL fragments ending within or just after the CDR3. In some embodiments, Cathepsin L or/and D will also cleave an antibody between the first and second inter-chain disulfide linkage to yield a F(ab′). In some embodiments, Cathepsin D will cleave within or just below the heavy chain hinge region, giving a F(ab′)2 fragment, which may then be further cleaved for example between the VH and CH1 portions and/or between the VL and CL portions. In addition, in some embodiments, both proteases also cleave between CH2 and CH3 to yield CH2 and CH3 fragments and along the CH2 domain. Thus, in some embodiments, cleavage with one or both enzymes will result in VH- and/or VL-comprising fragments that comprise the respective CDR1, CDR2, and CDR3.

In some embodiments, cleavage conditions are optimized for greater cleavage efficiency at one or more locations. Cleavage efficiency (also termed digestion efficiency), when referring to digestion of a protein or antibody generally, refers to the percent of the starting antibodies that are cleaved. Cleavage efficiency at a particular location or site, such as after the VH and VL of an antibody, may also be assessed. In some embodiments, a relatively low cleavage efficiency at each expected cleavage site is acceptable, such as of at least 1% to 50%, for example, in order to produce a range of fragment sizes. In addition, when there is a relatively a large amount of the antibody to be analyzed, a low overall cleavage efficiency is acceptable. In other cases, a higher cleavage efficiency, particularly for the site between the VH and CH and the site between the VL and CL for example, is desirable in order to maximize production of VH and VL antibody fragments. In some embodiments, the cleavage efficiency overall is at least 50%, at least 60%, at least 70%, at least 80%, at least 90%, at least 95%, at least 98%, or at least 99%, or 100%. In some embodiments, the cleavage efficiency for cleavage after VH and VL is at least 50%, at least 60%, at least 70%, at least 80%, at least 90%, at least 95%, at least 98%, or at least 99%, or 100%. The cleavage efficiency may be influenced by factors such as time of the reaction, temperature, pH, and presence of organic solvent, as well as the tertiary structure of the antibody.

The present disclosure encompasses cleavage reactions with cathepsin D and/or L that produce one or more fragments including VL, VH, CL, CH1, CH2, CH3, CH2-CH3, CL+CH1 (bonded), F(ab′), and F(ab′)2, as well as the resulting compositions comprising those fragments. Thus, for example, the enzymatic cleavage reactions herein may produce VL and/or VH fragments that comprise 90-150 amino acids in length, such as 95-140 amino acids, 100-140 amino acids, or 100-120 amino acids in length. Such VH and/or VL fragments may have molecular masses of, for example, 10-16 kDa, such as 10-13 kDa, 10-12 kDa, 10-11 kDa, or 11-12 kDa. Depending on the cleavage efficiency at various locations in the native antibody, larger fragments of 200-250 amino acids in size (e.g., bonded light chain and heavy chain fragments) may also be generated. F(ab′) and/or F(ab′)2 fragments may also be generated in some embodiments. In some cases, a mix of all of these fragments may result. Thus, some cleavage reactions may result in a composition comprising at least 2 of the following 10 types of fragments: VL, VH, CL, CH1, CH2, CH3, CH2-CH3, CL+CH1 (bonded), F(ab′), and F(ab′)2. Some cleavage reactions may result in a composition comprising at least 3 of the following 10 types of fragments: VL, VH, CL, CH1, CH2, CH3, CH2-CH3, CL+CH1 (bonded), F(ab′), and F(ab′)2. Some cleavage reactions may result in a composition comprising at least 2 of the following 4 types of fragments: VL, VH, CL, CH1, CH2, CH3, CH2-CH3, CL+CH1 (bonded), F(ab′), and F(ab′)2. Some cleavage reactions may result in a composition comprising a VL and/or a VH fragment of, e.g. 90-150 amino acids as well as at least 2 of the following further fragments: CL, CH1, CH2, CH3, CH2-CH3, CL+CH1 (bonded), F(ab′), and F(ab′)2.

For example, in some embodiments, the reaction is conducted at pH 2-8, pH 2-7, pH 2-6, pH 2-5, pH 3-6, pH 3-5, pH 3-4, pH 4-5, pH 2, pH 2.5, pH 3, pH 3.5, pH 4, pH 4.5, pH 5, pH 5.5, pH 6, pH 7, or pH 8. In some embodiments, the reaction is conducted at pH 3-5, such as at pH 3, 3.5, 4, 4.5, or 5. In some embodiments, the reaction is conducted at pH 4. Choice of pH may also depend on whether cathepsin L, cathepsin D, or both cathepsin D and L are to be used.

In some embodiments, the reaction is conducted at room temperature (which is about 18-25° C.) to 50° C. In some embodiments, the reaction is conducted at a temperature of 30-50° C., 30-45° C., 30-37° C., 37-45° C., 40-50° C. or 37-50° C. Higher temperatures, for example, may allow for shortening of the reaction time. In some embodiments, reactions are run for 4-30 hours, such as 4-12 hours, 12-24 hours, or 12-18 hours. In some embodiments, the reaction is conducted at pH 3-5, such as at pH 3, 3.5, 4, 4.5, or 5, and at 37-50° C. In some embodiments the reaction is conducted at pH 4, 37° C., for 12-24 hours.

In some embodiments, the cathepsin D is human cathepsin D, such as comprising an amino acid sequence of SEQ ID NO: 9. In some embodiments, the cathepsin L is human cathepsin L, such as comprising an amino acid sequence of SEQ ID NO: 10. In some embodiments, cleavage reactions are performed with a 1:20 to 1:2000 ratio of cathepsin D and/or L enzyme to protein. In some embodiments, a ratio of 1:20 to 1:500, 1:50 to 1:500, 1:100 to 1:500, 1:200 to 1:1000, 1:200 to 1:2000, 1:500 to 1:2000, 1:1000 to 1:2000, or 1:20, 1:50, 1:100, 1:200, 1:300, 1:400, 1:500, or 1:1000 is used. Varying the ratio may, in some embodiments, vary the extent of the cleavage, and may, in certain cases, impact the efficiency of different cleavage sites. For example, without being bound by theory, reducing the amount of enzyme might not only result in lower overall cleavage efficiency, but may also affect the resulting cleavage products by prioritizing certain, more accessible cleavage sites to others. Thus, in some embodiments, altering the ratio of enzyme to protein can impact the distribution of the resulting cleavage products.

Data herein unexpectedly showed that cathepsin L and cathepsin D cleavage is dependent on the tertiary structure of the antibody. For example, the cleavage efficiency at certain sites can vary depending upon the fold of the molecule near those sites or due to the disulfide bonding pattern. Tertiary structure may block cleavage at locations that would be cleaved in a denatured antibody. For example, as shown in FIG. 8, a cathepsin D cleavage occurs below the hinge region of a human IgG1 antibody, but that cleavage does not occur in a human IgG2B or IgG4 antibody due to differences in disulfide bonding patterns.

Thus, in some embodiments, the antibody is cleaved in the native state. An antibody in the “native state” is an antibody that retains its general, native folded structure and disulfide bonding, i.e., the antibody is not denatured or unfolded, and disulfide bonds are not reduced. In contrast, a bottom-up cleavage is typically performed by digesting the protein to be analyzed in a denatured state so that small peptides may readily be generated from the protein. In some embodiments, as noted above, organic solvent may be added up to 50%, for example to relax the antibody state to some extent for optimal cleavage, but to avoid denaturing or unfolding the antibody so that the antibody remains in the native state. For example, in some embodiments, 0-30% organic solvent may be added, such as 5-30%, 10-30%, 0-10%, 5-15%, 10-20%, 15-25%, or 20-30%. The organic solvent may comprise, for example, acetonitrile, methanol, ethanol, or isopropyl alcohol. In some embodiments, the reaction buffer comprises no more than 50% organic solvent. In some embodiments, the reaction buffer comprises no more that 30% organic solvent. In some embodiments the reaction buffer comprises no more than 10% organic solvent. Again, as also noted above, in some embodiments, the antibody has not been reduced so as to avoid interfering with its natural disulfide bonding pattern.

In some embodiments, where both cathepsin D and cathepsin L are to be used, the enzymes are exposed to the antibody one after the other, while in other embodiments they are incubated with the antibody simultaneously, under the same reaction conditions. In some embodiments, both enzymes are added simultaneously to the antibody in a buffer at pH 3-5, such as at pH 3, pH 3.5, pH 4, pH 4.5, or pH 5, and at a temperature of 30-50° C., such as at 37° C. In some such cases, 10-30% organic solvent is also added to the reaction mixture. Unexpectedly, up to 100% cleavage efficiency between the VL and CL and between the VH and CH of antibodies (e.g., at least 50%, at least 60%, at least 70%, at least 80%, at least 90%, at least 95%, at least 98%, or at least 99%, or 100%) was observed when both cathepsin D and L were used simultaneously in a buffer at pH 3-5, such as at pH 3, pH 3.5, pH 4, pH 4.5, or pH 5, and at a temperature of 30-50° C.

In some methods herein, for example, because the cathepsin L/D cleavages appear to be tertiary structure dependent, the antibody can remain in the native state during the cleavage. In some embodiments, the antibody remains in the native state during cleavage and also after cleavage and during MS analysis. In some methods herein, the antibody is not treated with denaturing agents or agents that reduce disulfide bonds during or after the cleavage. In some methods herein, the antibody retains its disulfide bonding during and after the cleavage. Some methods herein comprise performing mass spectrometry (MS) analysis of one or more antibody fragments following the cleavage, wherein the antibody remains in the native state and is not treated with denaturing agents or agents that reduce disulfide bonds prior to the MS analysis. Therefore, in some embodiments, in addition to the favorable selection of antibody fragments that reaction with cathepsin L and/or D may produce, such as VL, VH, F(ab′), F(ab′)2, or other fragments comprising CDR1, CDR2, and CDR3 of a heavy or light chain, one may perform MS analysis directly following cleavage without denaturing or reducing the disulfide bonds in a resulting antibody fragment.

Following cleavage of the antibody, the antibody may be treated to remove the enzyme or enzymes and also to change the buffer, and/or to isolate particular fragments.

### Optional Treatments Following Cleavage

The antibody fragments generated during cleavage may be further treated in a variety of ways before mass spectrometry analysis.

In some embodiments, the antibody fragments may be treated to reduce disulfide bonds. Upon reduction of disulfide bonds, certain fragments comprising two polypeptide segments joined by a disulfide bond, for example, may dissociate into two separate species. In some cases, a portion of the cleaved antibody may be assessed by mass spectrometry without reducing S—S bonds while another portion may be reduced before it is assessed, for instance, allowing for top-down analysis of both the reduced and unreduced fragments so as to obtain further data. In some embodiments, the resulting fragments may be alkylated or otherwise chemically modified.

In some embodiments, following cleavage, the resulting fragments may be isolated from the enzymatic reaction mixture in various ways. For example, the fragments can be isolated by chromatography or size filtration. For example, fragments may be separated via capillary electrophoresis or liquid chromatography. Or fragments may be separated from other components by filtration, such as using a molecular weight cut off filter that retains higher molecular weight species but allows smaller species, such as certain cleaved fragments, to flow through. In some embodiments, for example, if desired fragments are, for example, below 25 kDa, such as about 10-14 kDa, a 20 kDa or 30 kDa molecular weight cut-off filter, for example, could be used to separate desired fragments from larger fragments, with this step optionally followed by a concentration step of the flow through on a MW filter that retains the fragments of interest. If desired, a relatively low molecular weight cut-off filter could also be used to separate desired fragments from smaller fragments or buffer contaminants, e.g. a filter that retains molecules above 3 kDa or above 10 kDa.

The cleaved antibody fragments may also be isolated from the enzymatic reaction mixture by exchanging the buffer, for example, to alter pH and other buffer conditions. This can occur during a chromatography or filtration process, for example. In some embodiments, fragments may be analyzed by Edman degradation, optionally in combination with further enzymatic or MS cleavage, as an alternative means to obtain their sequence or to verify their sequence.

In embodiments herein, any combination of the above treatments may be used.

Mass Spectrometry Analysis

In order to obtain sequence or structural information about the antibody fragments, mass spectrometry analysis may be performed on at least one of the fragments. In some embodiments, MS analysis is performed on the VH and/or VL fragments obtained from cleavage with cathepsin L or D or both cathepsin L and D. In some embodiments, a top-down analysis method is performed on at least one of the generated fragments. In some embodiments, a bottom-up approach is performed. In some embodiments, a combination of top-down and bottom-up approaches are used.

In some embodiments, a complete sequence of at least one of the fragments is obtained. In other embodiments, the sequence of an at least 10 amino acid stretch is obtained. In some embodiments, the sequence of an at least 15 amino acid stretch is obtained. In some embodiments, the sequence of an at least 25 amino acid stretch is obtained. In some cases, the sequence of an at least 50 amino acid stretch is obtained. In some cases, the sequence of a stretch of 10-50, 15-50, 25-50, or 10-25 amino acid stretch is obtained. In some cases, the CDR3 sequence is obtained for a VH or VL fragment. In some cases, the CDR1, CDR2, and/or CDR3 sequence is obtained for a VH or VL fragment. In some embodiments, the sequence of the antibody fragment was unknown prior to the method, and thus, the method obtains the sequence or partial sequence of that fragment de novo.

MS measures the mass to charge ratio (m/z) of one or more molecules in a sample. Tandem MS may be used in some embodiments, and is a process by which a single ion, multiple ions, or the entire mass envelope (the precursor(s)) are moved to a fragmentation chamber and the fragmented products are then sent to a mass analyzer. Depending on the design of the mass spectrometer, the fragmentation event can happen before a single mass analyzer, between two or multiple different analyzers, or within a single mass analyzer.

MS analysis may have a variety of options. In some embodiments, the MS instrument does not comprise a quadrupole. In some embodiments, the MS instrument comprises at least one quadrupole. In some embodiments, the MS instrument comprises at least 2 quadrupole analyzers. In some embodiments, the MS instrument comprises at least 3 quadrupole analyzers. In some MS's, the detector is an ion trap, quadrupole, Orbitrap™, or time of flight (TOF). In some embodiments, the MS instrument or method is multiple reaction monitoring (MRM), single ion monitoring (SIM), triple stage quadrupole (TSQ), quadrupole/time of flight (QTOF), quadrupole linear ion trap (QTRAP), hybrid ion trap/FTMS, time of flight/time of flight (TOF/TOF), Orbitrap instruments, ion trap instruments, parallel reaction monitoring (PRM), data dependent acquisition (DDA), data independent acquisition (DIA), multi-stage fragmentation or tandem in time MS/MS. In some embodiments, the mass spectrometer comprises at least one quadrupole and uses a means of dissociation chosen from collision induced dissociation (CID), higher energy collisional dissociation (HCD), electron capture dissociation (ECD), electron transfer dissociation (ETD), electron transfer/higher energy collisional dissociation (EThcD), or photodissociation such as UV photodissociation (UVPD).

In some embodiments, direct infusion or static spray infusion or flow-injection-analysis mass spectrometry is used to analyze one or more antibody fragments. In some cases, infusion is performed directly after the enzymatic cleavage reaction, or following a buffer-exchange step following the cleavage reaction. In some cases, for example, infusion is an alternative to isolating a desired fragment via techniques such as chromatography followed by mass spectrometry such as LC-MS. In infusion methods, the sample comprising the antibody fragment or fragments may be input directly into a mass spectrometer instrument for fragmentation via electrospray or nanospray ionization. The precursor ions may then be optionally separated by a quadrupole mass filter for mass measurement and analysis and fragmented for amino acid sequence prediction.

Top-Down and/or Bottom-Up Analysis

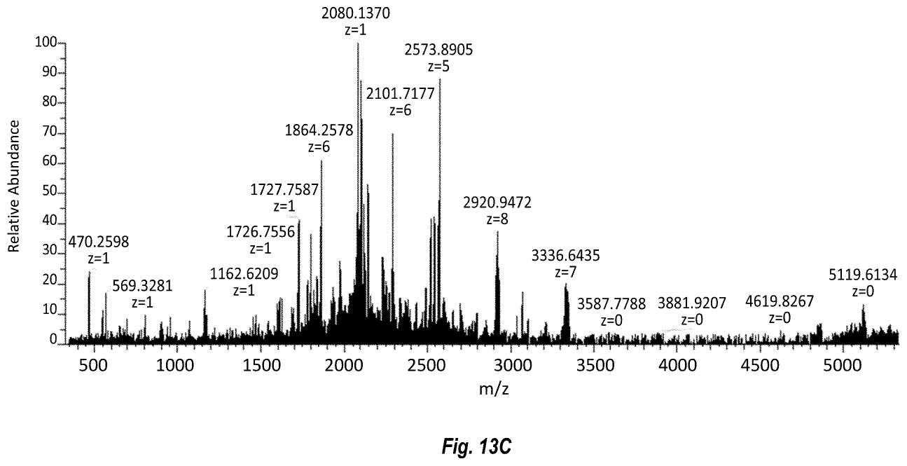

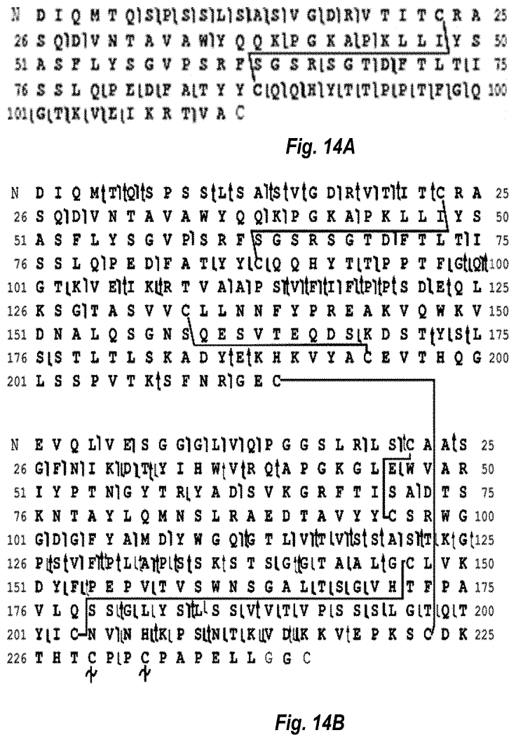

In some embodiments herein, the mass spectrometry is used for a top down sequence analysis. For example, the mass spectrometer may be used to further fragment the cleaved antibody fragments into a series of product ions for which sequence predictions may be made. An example is shown, for instance in Table 13 below. Associated software may be used to align predicted sequences. An example is shown, for instance, in FIGS. 14A and 14B. In the case of a VH or VL fragment, for example, since those fragments typically begin at the natural N-terminal amino acid of the antibody to be analyzed, mass spectrometry sequence information may be deconvoluted and aligned starting from the N-terminal of the fragment. Thus, the N-terminal may serve as a reference point for alignment of product ions generated during mass spectrometry. For example, when analyzing a VH or VL, since those fragments are at the N-terminal end of an antibody polypeptide chain, their product ions generated in a MS analysis can be aligned from the N-terminal end. For this and other reasons, it is not necessary for the enzyme to specifically cleave at a single amino acid to form the C-terminal end of the fragments.

Various software packages may be used to deconvolute and align sequence information from a top-down MS analysis. Examples include PEAKs™ (Bioinformatics Solutions, Inc.) and Byos™ and its component software (Protein Metrics, Cupertino, Calif., USA).

In some embodiments, the methods herein may be combined with additional middle down methods as well as bottom up methods for further sequence analysis. For example, cleavage with cathepsin L and/or D may be combined with cleavage by other enzymes that cleave in or near the hinge region of an antibody, such as protease Streptococcus pyogenes (IdeS) (e.g. FabRICATOR™ (Genovis, Inc.)) or GingisKhan™ (Genovis). In some embodiments, cathepsin L, cathepsin D, or a combination of cathepsin L and D may be combined with IdeS or GingisKhan™ (Genovis). In some embodiments, other proteases such as trypsin, papain, chymotrypsin or others may be combined with cathepsin L, cathepsin D, or both cathepsin L and D. The products from such cleavage reactions may then be analyzed by top down or bottom up processes, for example.

In some embodiments, fragments generated from cathepsin L and/or D cleavage reactions may be further analyzed by bottom up methods. Thus, in some embodiments, for example, resulting fragments may be reduced or denatured and then further cleaved with proteases such as trypsin or papain to generate smaller fragments for MS analysis. In some embodiments, a top down analysis may be combined with a bottom up analysis, for example, as a means of validating and cross-checking sequence information.

De Novo Antibody Sequencing Workflows

In some embodiments, methods herein may be used for partial or complete de novo sequence analysis of one or more antibody fragments. In general, determining a stretch of sequence de novo requires complete fragmentation of that stretch of sequence during mass spectrometry analysis, i.e., fragmentation after each subsequent amino acid in the sequence stretch, so that the fragmentation allows the mass of each successive amino acid residue may be determined, and therefore, the amino acid residue to be identified. Depending on the fragmentation method chosen, reduction of disulfide bonds may be performed before mass spectrometry analysis in order to ensure complete fragmentation of a particular stretch of amino acids. For example, if an Orbitrap™ with higher energy collisional dissociation (HCD) is used for fragmentation, reduction may be required to obtain fragmentation after each successive residue. In other cases, de novo sequence determination may be performed without prior reduction of disulfide bonds. In other embodiments, a portion of the sample containing the antibody fragment can be analyzed without reduction and another portion of the sample can be analyzed after reduction.

For example, FIG. 14A shows a depiction of a trastuzumab light chain variable region following MS fragmentation. As shown in that figure, a span of continuous y ions occurs at residues H91-E105. Thus, the sequence of this portion of the VL, which roughly corresponds to the CDR3 region, could be determined by top-down analysis. Such a top-down analysis can use commercially available software to determine amino acid sequence from mass data and alignment of fragments, such as PEAKS™ (Bioinformatics Solutions, Inc.) or Supernovo™ (Protein Metrics), or open source programs. To determine the sequence of additional portions of an antibody fragment such as a VL fragment, additional fragmentation frequencies may be used so that fragmentation occurs after all or nearly all amino acid residues in the fragment molecule. In addition, the antibody cleavage products may be treated to reduce disulfide bonds so that disulfide bonds do not interfere with fragmentation. To sequence larger segments of an antibody fragment, commercially available software parameters may be adjusted to allow de novo determination of sequences of, for example, up to about 12 kDa fragments. For example, the total peptide length allowed within the program may be extended, error tolerances allowed at the MS1 level may be increased, and incorporation of disulfide rules may be enabled, for example, with modifications to the p-score or A-score. Additionally, in some embodiments, candidate de novo sequences may be validated by determining the sequences using more than one software program.

Kits and Products

The present disclosure also encompasses kits and products for conducting optimized cathepsin L, cathepsin D, or cathepsin L and D cleavage reactions on antibodies. In some embodiments, a kit comprises cathepsin L. In some embodiments, a kit comprises cathepsin D. In some embodiments, a kit comprises both cathepsin L and cathepsin D. In some embodiments, a kit comprises one or more additional enzymes, such as IdeS, papain, trypsin, chymotrypsin, or GingisKhan™ in addition to the cathepsin L and/or D. In some embodiments, the kit comprises one or more reaction buffers for cathepsin L and/or cathepsin D cleavage reactions. In some embodiments, the kit comprises one or more reaction buffers at pH 2-8, pH 2-7, pH 2-6, pH 2-5, pH 3-6, pH 3-5, pH 3-4, pH 4-5, pH 2, pH 2.5, pH 3, pH 3.5, pH 4, pH 4.5, pH 5, pH 5.5, pH 6, pH 7, or pH 8. In some embodiments, the reaction buffer is at pH 3-5, such as at pH 3, 3.5, 4, 4.5, or 5. In some embodiments, the reaction buffer is at pH 4. In some embodiments, the reaction buffer comprises one or more organic solvents. In some embodiments, the organic solvent is methanol, ethanol, isopropyl alcohol, or acetonitrile. In some embodiments, the reaction buffer comprises 5-50% organic solvent, such as 5-30%, 10-30%, 0-10%, 5-15%, 10-20%, 15-25%, or 20-30%. In some embodiments, the reaction buffer comprises no more than 50% organic solvent. In some embodiments, the reaction buffer comprises no more that 30% organic solvent. In some embodiments the reaction buffer comprises no more than 10% organic solvent. In some embodiments, a product or kit also comprises instructions for use in conducting a cathepsin D and/or cathepsin L cleavage reaction on an antibody composition.

### EXAMPLES

### Example 1

Notwithstanding the huge successes of proteomics in protein sequencing, complete sequencing of antibodies (Abs) still presents considerable challenges [1, 2]. Performed with the goal of annotating and validating the location and order of every amino acid (and any variants), it currently requires that 4-5 different proteases are used to generate overlapping and ideal-length peptides for liquid-chromatography (LC)-mass spectrometry (MS) bottom-up proteomics analysis. This is a higher standard of analysis compared to a simple monoclonality check, performed with intact analysis, or protein verification, which commonly uses a single enzyme for digestion. If annotated de novo, these mass spectra must be processed through specialized Ab sequencing programs that use information on the extracted mass shifts between the product ion peaks. Assignments are made based on computational rules limiting the allowed mass error, making the success of such analyses highly dependent on the superb quality of the MS/MS spectra [3-7].

Alternatively, middle-down enzymatic proteomics approaches are starting to be explored, where an antibody is first cleaved above or below the hinge region, to generate Ab protein fragments amendable for sequencing by top- and/or middle down proteomics [8-10]. In these methods, the F(ab′)2, F(ab′), Fc, Fd, light chain (LC), and heavy chain (HC) fragments can be selectively generated depending on the protease or denaturant used, whereby especially the protease Streptococcus pyogenes (IdeS) (e.g. FabRICATOR™ (Genovis, Inc.)) has become quite popular [10-13]. IdeS is a protease that digests antibodies at a specific site just below the hinge, generating a homogenous pool of F(ab′)2 and Fc/2 fragments [12, 13].

The main difference of middle-down, when compared to bottom-up approaches for sequencing, is that it uses relatively higher molecular weight (HMW) precursors (5-25 kDa). These protein fragments precursors provide a corresponding sequence on which all fragment ions can and should be mapped. Combined with the MS1 intact information on the different fragments, particularly when determined at high resolving power, top- and middle-down proteomics approaches can be quite powerful [8-11, 14-21].

The combination of native mass spectrometry [22-28] with top-down proteomics [14, 16, 20, 29, 30] can offer additional advantages for the analysis of biotherapeutics. LC separations of digested complex mixtures are often insufficient, resulting in co-elution of species, and, under denaturing conditions, these masses often overlap, which reduces the signal-to-noise and limits accurate mass deconvolution. Likewise, while higher charge states result in increased higher collisional energy dissociation (HCD) fragmentation efficiency in top-down analysis, native mass spectrometry can allow for using large isolation widths, encompassing multiple charge states, when proteins are moved into a less crowded m/z space [31, 32]. This yields increased signal-to-noise of the product ions, higher coverage, and enables a simplified workflow. Generally, top-down of large intact proteins with non-reduced disulfide bonds (>25 kDa) lacks sufficient coverage for de novo applications on current MS “workhorse” instrumentation found in industry, which are traditionally time of flight with collisional induced dissociation or Orbitrap with HCD instruments [31]. These lack electron induced dissociation, which is a highly efficient and orthogonal fragmentation approach [14, 18, 33-36], and ultraviolet photo dissociation, which is also suitable for very large polypeptides [16, 17, 30]. Without multiple fragmentation methods, incomplete coverage will limit top- and middle-down applications. Thus, there is an unmet need to establish more and alternative middle-down workflows, using heretofore underexplored proteases, that yield protein fragments more suited to standard LC-MS/MS HCD experiments.

In the field of IgG analysis, the most commonly used middle down proteolytic enzymes are the aforementioned IdeS, which cleaves preferentially at a single site below the hinge region [12, 13], and GingisKHAN™ (Genovis, Inc.) [37], which cleaves just above the hinge. Most alternative middle-down digestions have attempted to control the rate of promiscuous enzymatic or chemical degradation, such that the size of the polypeptides is tied to the exposure time of the protein to the enzymes. Most recently, Aspergillus saitoi acid proteinase was immobilized on an electrospray emitter for applications in online peptide mapping [38, 39]. In the exploratory work, peptides of 3-15 kDa were generated during 0-2 min exposure times at a ratio of 1:5. Likewise, pepsin activity has been intentionally restricted, through de-optimization of the pH, to yield middle-down size fragments and the F(ab′)2 domain [40]. Also, peptides generated by the proteases OmpT [41] and Sap9 [42] generally range from 1.5-15 kDa in size, which are larger than the average tryptic peptide size.

The commercially available lysosomal endopeptidases Cathepsin L [43, 44] and D [45] have been reported in the literature as two proteases that have unpredictable cleavage sites. Cathepsins represent a family of enzymes found in the lysosome that are responsible for protein degradation through hydrolysis of the protein backbone [46]. Cathepsin L is a member of the peptidase C1 family and is reported to cleave after F,R or R,R sites at P2 and P1 [47-52], and Cathepsin D, an aspartyl proteinase, is reported to cleave between hydrophobic residues and especially Leu and Phe, however these preferences are contradicted throughout the cited literature [47, 53-57]. These studies have been conducted primarily on protein standards, extracts and peptides by SDS-PAGE, protein sequencers, or peptide substrate microarrays. One consistency across these studies is that despite the enzymes' promiscuity, they yield a surprisingly limited number of fragments. Protease degradation rates vary widely depending upon the enzyme isoform, protein sequence, secondary and tertiary structure, buffer components (especially metals such as copper) and storage conditions [58].

The evaluation of new enzymes, especially when given few digestion site restrictions, is challenging because of the large number of internal fragments that can be generated at any location and any size found in a targeted protein. This large computational space is further expanded when including possible disulfide linkages between chains or post translational modifications, such as the glycans found on the Fc. The work herein described, to specifically map the Cathepsin digestion of Abs, was enabled through utilization of the Intact Mass™ algorithm [59], which was updated in 2019 to include an automated annotation feature for clipped species. This algorithm is described in further detail in the Methods section.

Here, we set out, using a single standard digestion condition, to explore the cleavage sites of Cathepsin L and D when targeting three IgG1 antibodies (trastuzumab, rituximab, and obinutuzumab), one IgG2/4 antibody, eculizumab, and one IgG1 bispecific antibody comprising a knob-into-hole modification (anti-Her2/anti-CD3). The Ab fragments formed were directly analyzed by high resolution native mass spectrometry and by denaturing LC-MS intact analysis. The cleavage sites of Cathepsins L and D were explored under different pH, temperature, and denaturing (percent organic) conditions, and subsequently optimized to maximize the cleavage between the variable and constant regions of the Ab. Our most interesting finding is that we can specifically generate a ≈12 kDa fragment encompassing the complementarity determining region (CDR) region of the Ab light chain. As far as we know, this is the first demonstration of a middle down technique directly targeting the highly variable region of an Ab, and by fragmenting this 12 kDa fragment with standard HCD, we could maximize the coverage over this region.

### A. RESULTS

A.1. Mapping Cathepsin L and Cathepsin D Cleavage sites Across Class of Antibodies

The cleavage sites of Cathepsin L and Cathepsin D were evaluated over five antibodies (Abs) to look for the common digestion motifs and to evaluate differences between the IgG1 and the IgG2 sub-type. As far as we know, this is the first report of Cathepsin L and D digestion of intact Abs, and the first concerted effort to look at sites across those of similar or dissimilar homologies.

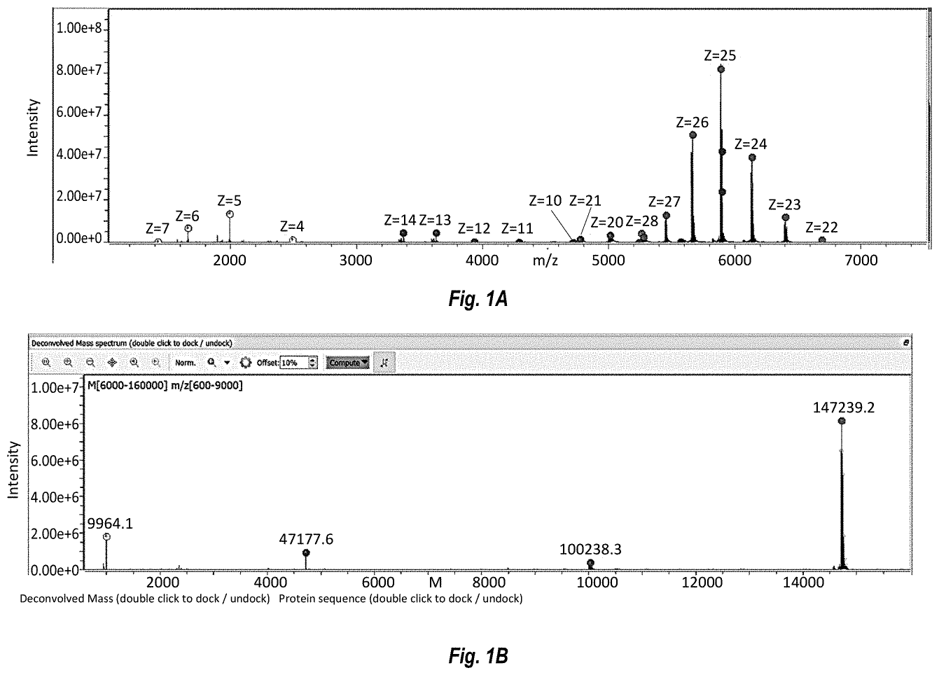

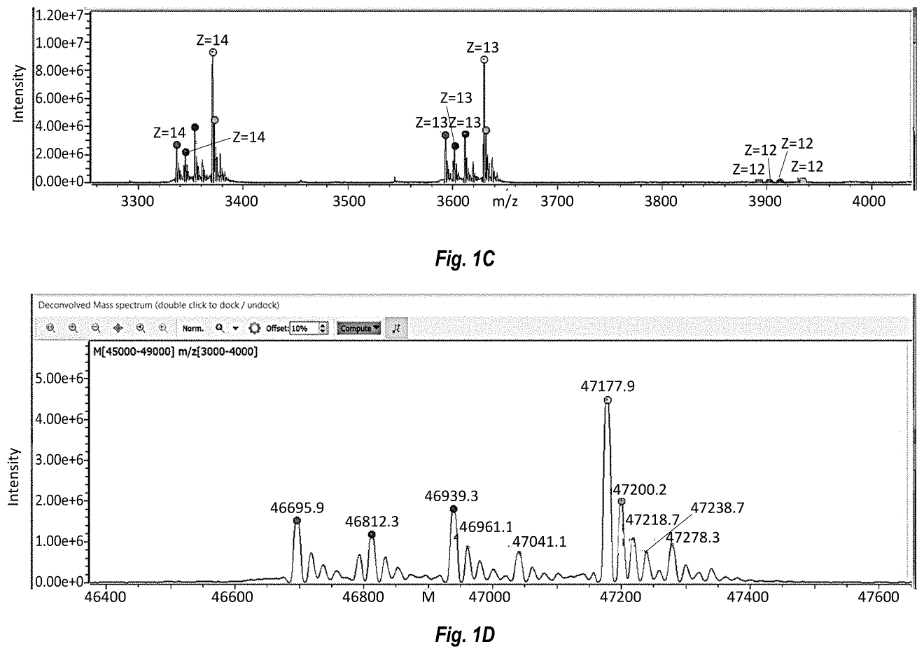

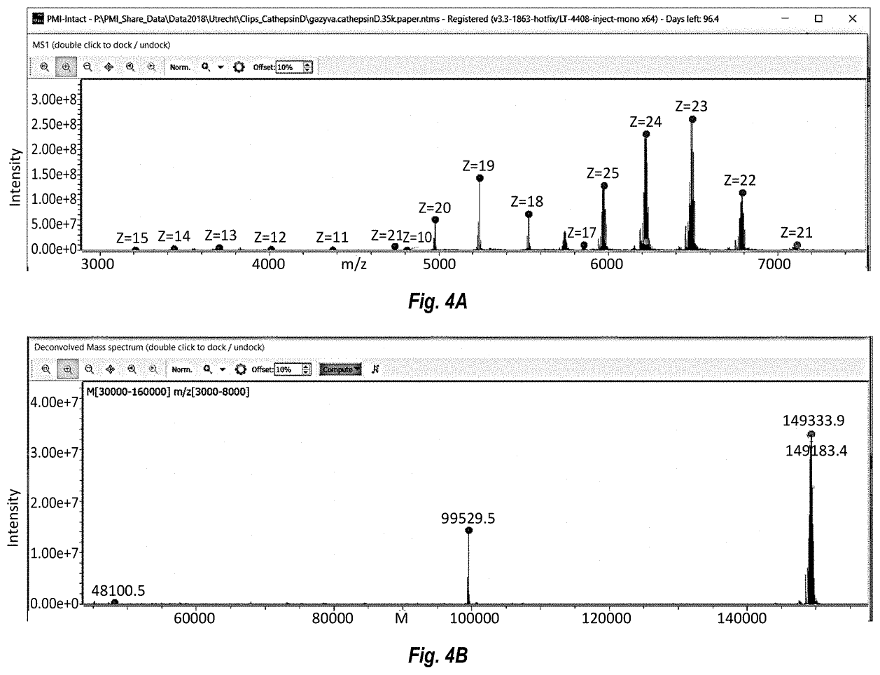

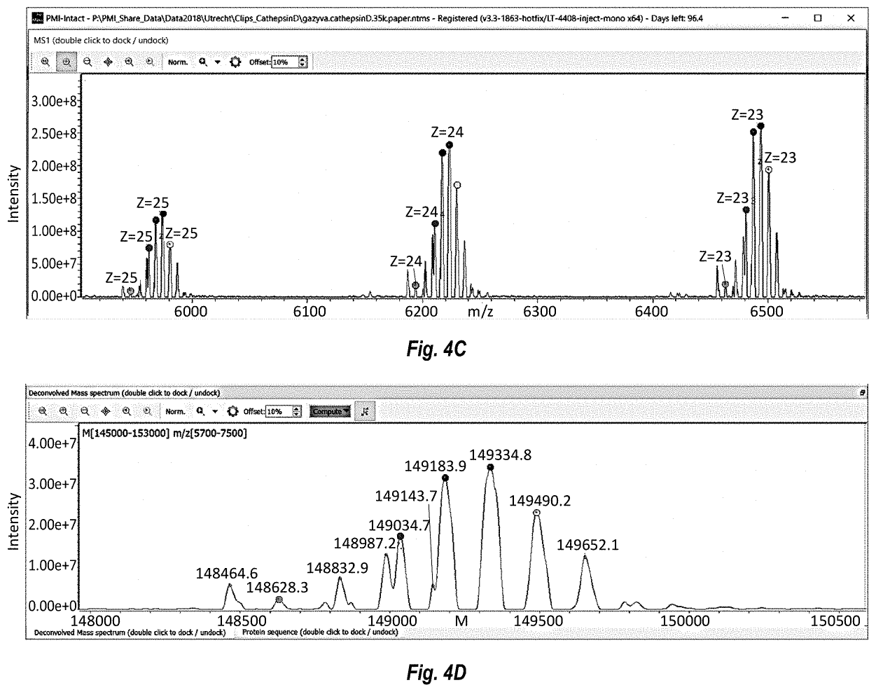

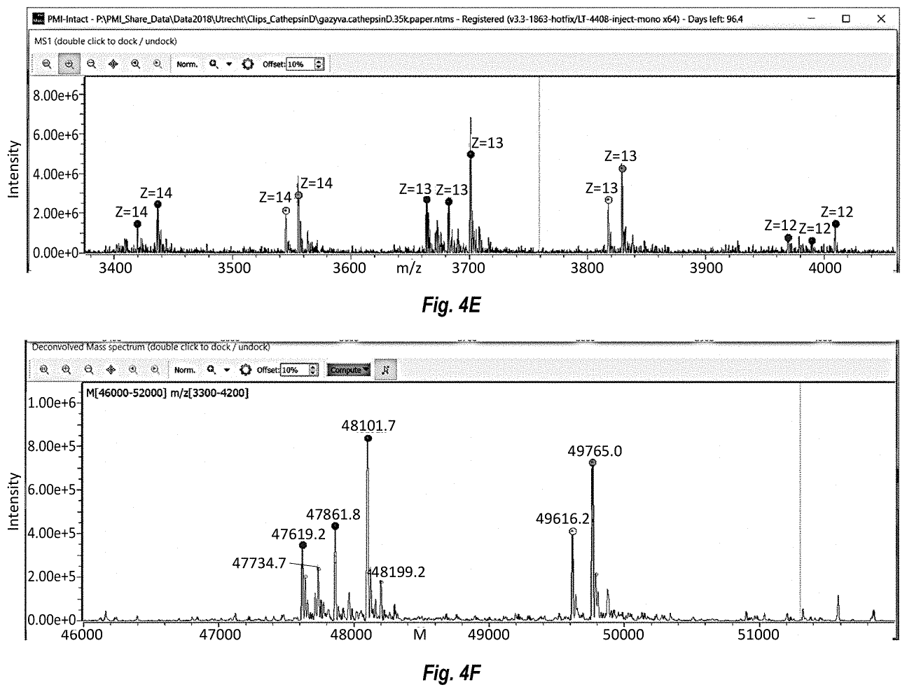

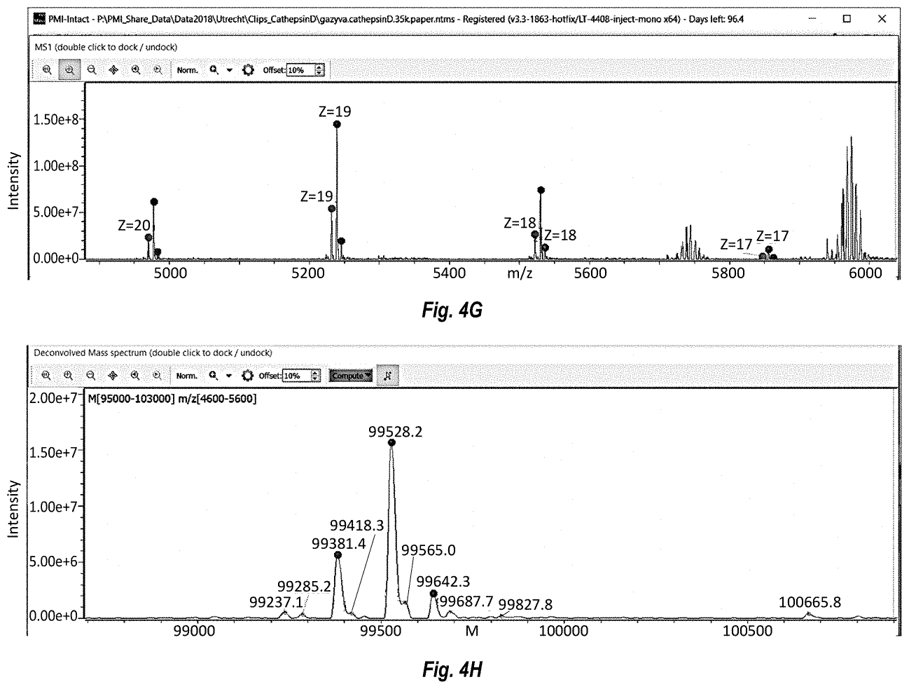

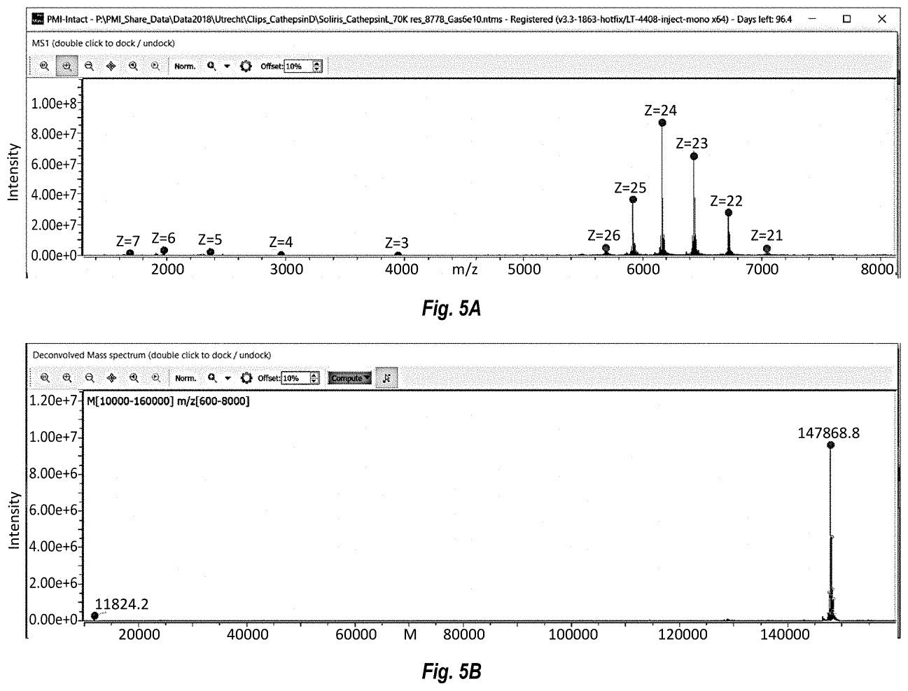

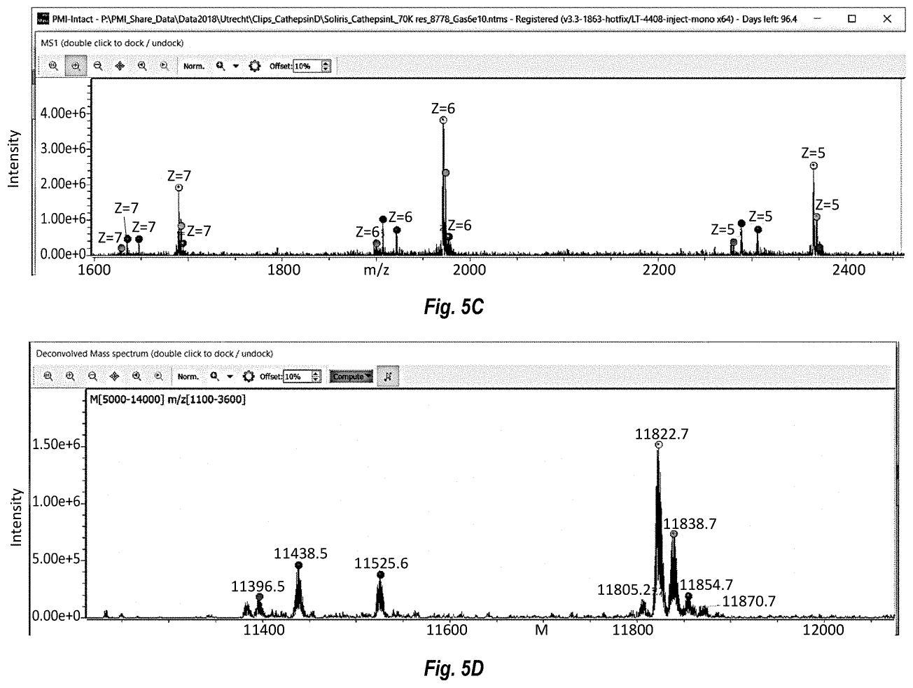

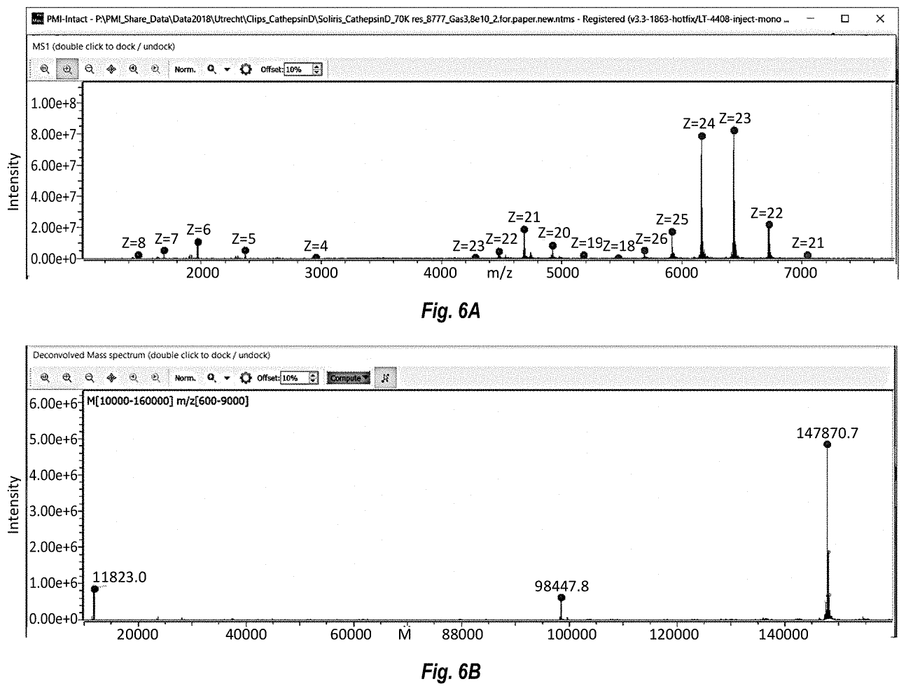

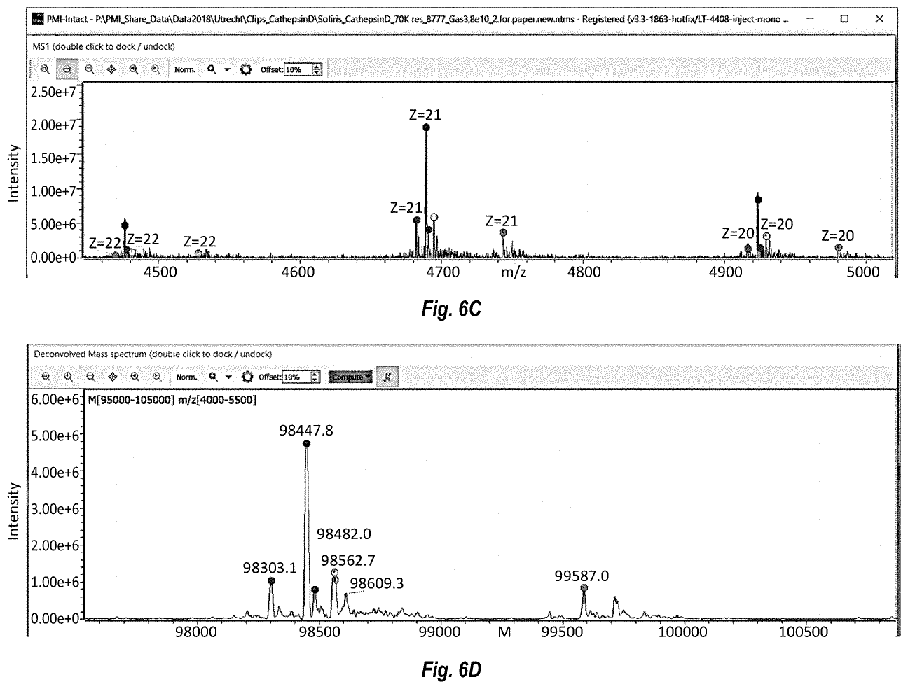

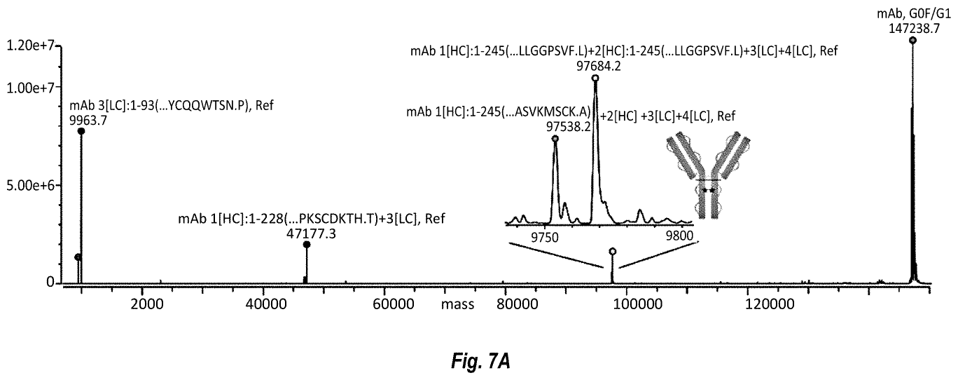

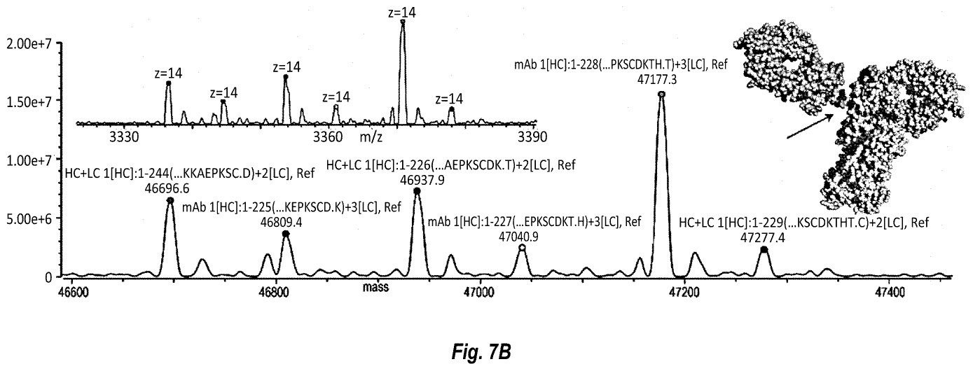

Identification of the protein fragments found in the digest were made using Intact Mass and validated manually. The deconvolved protein fragment peaks ranged from 9-98 kDa in molecular weight for both Cathepsin proteases and across all Abs (Tables 1-9, FIGS. 1-6). An example of the automated assignments for rituximab when cleaved by Cathepsin D is given in FIG. 7 and FIG. 1. At the initially picked conditions (pH 7, 2 days incubation), the enzymatic efficiency of Cathepsin L and D was found to be rather low ˜1%. While the size distribution of the protein fragments spanned a large molecular weight (MW) range, only four primary species were observed, which corresponded to cuts between the VL and CL, the VH1 and CH1, the CH1 between the 1^{st} and 2^{nd} inter-chain bonds, and immediately after the hinge region (at a near-identical location to the IdeS cleavage site). At lesser abundance, multiple cleavages throughout the HC were observed (Table 1).

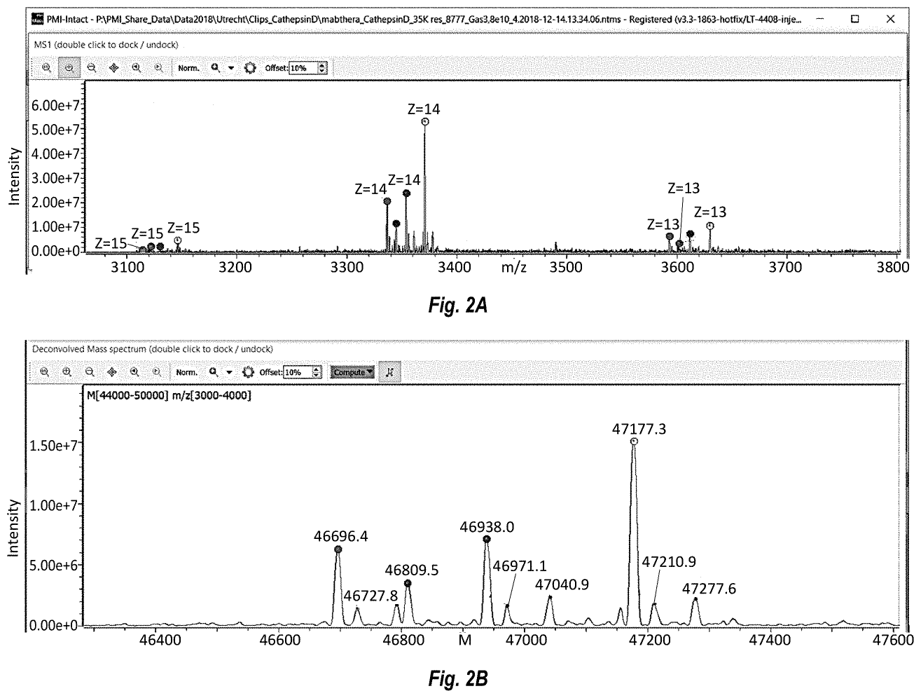

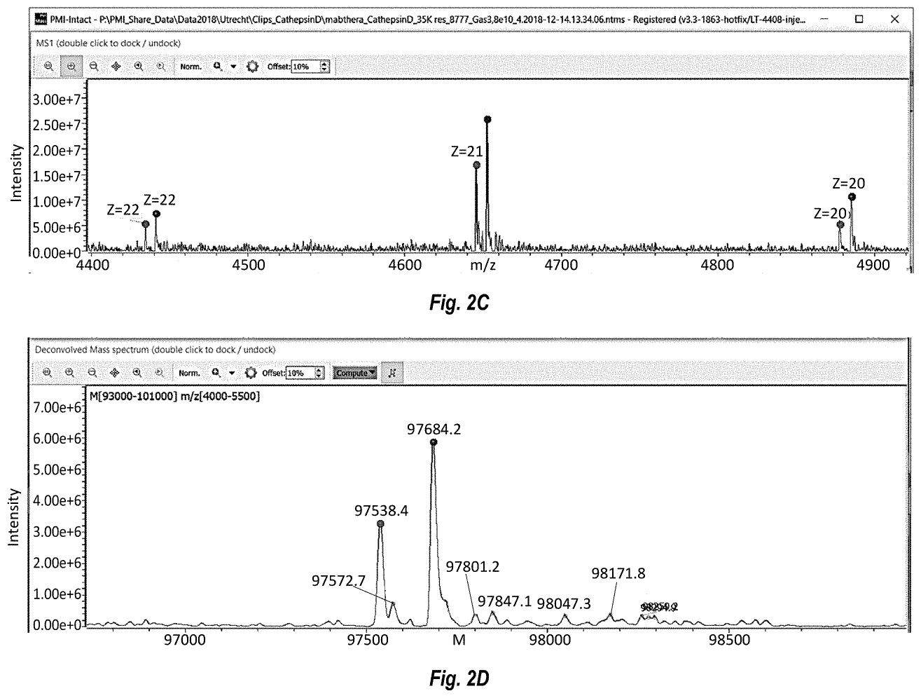

Note that the masses shown in FIG. 1 may vary slightly from the masses given in the tables disclosed herein (for example, 9964.1 above is the same peak as 9963.45 in Table 2), because the exact average masses depend upon the time, mass, resolution, signal-to-noise, and m/z ranges, as well as other parameters, used in charge deconvolution. Similarly, the masses in FIGS. 2-6 also may vary slightly from the masses given in the tables disclosed herein because the exact masses depend upon the time, mass, and m/z ranges, as well as other parameters, used in charge deconvolution.

When including all low-intensity signals, a large number of total peaks were deconvolved, and this reflected localized, versus global, cleavage site diversity. For example, in FIG. 7B, it is shown that Cathepsin D may cleave at approximately 14 different but sequential amino acids to generate the F(ab′), with many of them generated with a near-equal likelihood based on their relative signal intensity (Tables 2 and 3). In this particular region, cleavage was shown to occur after C, D, K, T, or H. Further reflecting the local cleavage diversity, certain products were found to be the result of an asymmetric clip, versus a simple single-site cleavage. For example, as shown in FIG. 7A, the peak at 97,538 Da is assigned to the complex of 2 LC, plus one heavy chain (E1-F244) that was clipped with 1 amino acid difference to the second HC (E1-V243). Support for asymmetric cleavage assignments is provided in the top-down analysis section. Considering that there were no amino acid enrichments found across all identified peptides within the −4 to +4 cleavage site motifs (Tables 1-3), this lent itself to the hypothesis that the Cathepsin D specificity could be influenced by the secondary and tertiary structure as much as the primary sequence, and this role will be discussed during the digestion optimization experiments. As with FIGS. 1-6, the masses shown in FIG. 7 may vary slightly from the masses given in the tables disclosed herein because the exact masses depend upon the time, mass, and m/z ranges, as well as other parameters, used in charge deconvolution.

FIG. 8 provides a graphical overview of the locations of the clips observed when mapped onto schematics of antibodies with IgG1 and IgG2-B disulfide bonding patterns. Although the site of the clips varied with the Ab and the Cathepsin protease used (Tables 1-9), a few rules held without exception across all IgG1s: (1) Cleavage occurred directly after the HC and LC CDR3 by Cathepsin L and D; (2) Cathepsin D cut between the 1^{st} and 2^{nd} inter-chain disulfide to yield the F(ab′), but Cathepsin L did not; (3) the heavy chain hinge was prone to variable clipping by both proteases directly below the hinge region, giving the F(ab′)2 and (4) there were cleavage sites identified for both Cathepsin L and D between the CH2 and CH3.

When compared to the IgG1s, eculizumab demonstrated the same patterns of F(ab′) cleavages but distinctly lacked a cleavage site within the hinge. Thus, only the F(ab′)2 versus the F(ab′) was observed. This difference further supports the role of tertiary structure in mediating the digestion products of Cathepsin D and L.

### A.2. Quantitative Optimization of Digestion Efficiency and Assignment of Fragments

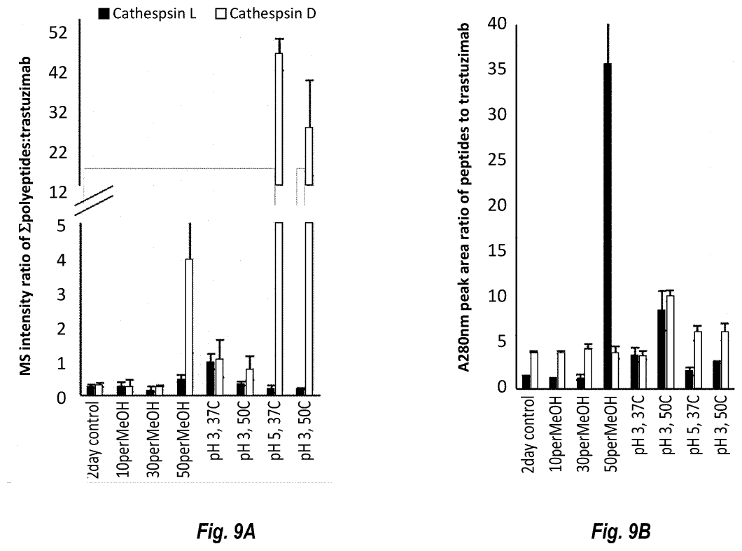

Although it was unpredictable whether Cathepsin D and L would cleave proteins at specific sites, or whether these enzymes were more promiscuous, we found that Cathepsin L and D will specifically cleave Abs at four primary sites. This specificity showed high potential for use of Cathepsin D and L in a middle down sequencing workflow. However, the digestion efficiency was rather poor under the standard conditions. To improve digestion efficiency, we selected pH as a digestion variable [60, 61]. Likewise, as the tertiary structure of the Ab was thought to influence cleavage, the level of organic solvent was also evaluated. Lastly, a temperature of 37° C. or 50° C. was tested for digestion. Three replicates were performed and the ratio of the summed intensities of the intact trastuzumab charge states to the digested fragments were compared (FIG. 9A). LC-UV-MS was used to minimize ion suppression from co-ionized species, compared to nESI infusion, and improve the relative quantitation. Quantitation was also done by taking the ratio of UV peak areas summed over the polypeptide elution regions versus the Ab peak (FIG. 9B) to ensure the results were consistent.

It was determined that the optimal pH efficiency for Cathepsin L was pH 3 and for Cathepsin D at pH 5 (see FIGS. 9A and 9B), with trends supported by the MS and UV data. Runs at 50% methanol showed increased efficiency for both enzymes, but had significantly increased variability compared to the other conditions, likely due to the relative instability of the Ab under these conditions. Physical observations had showed cloudiness in some of the 50% methanol samples, and it is possible that precipitation of the Ab, but solubilization of the protein fragments, resulted in inflated digestion ratios and increased variability. At lower methanol levels (10 or 30%), no differences in digestion efficiency compared to the control were observed by MS or UV. No conclusions could be made on the effects of temperature, with data from the UV and MS directly contradicting each other for the Cathepsin L and D data. Across all conditions, the protein fragments observed were in good agreement with those reported in the IgG1 control studies. For example, the mass at 11,197 Da was detected across all the IgG1 samples and is a low abundant species (<1 e4) that corresponds to HC peptide E1-D102.

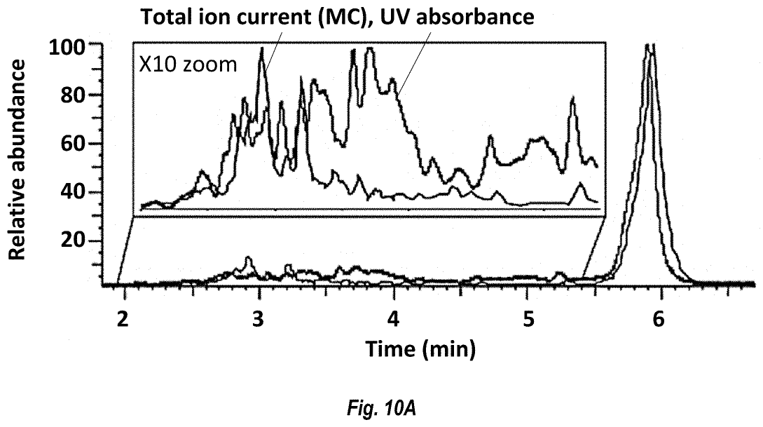

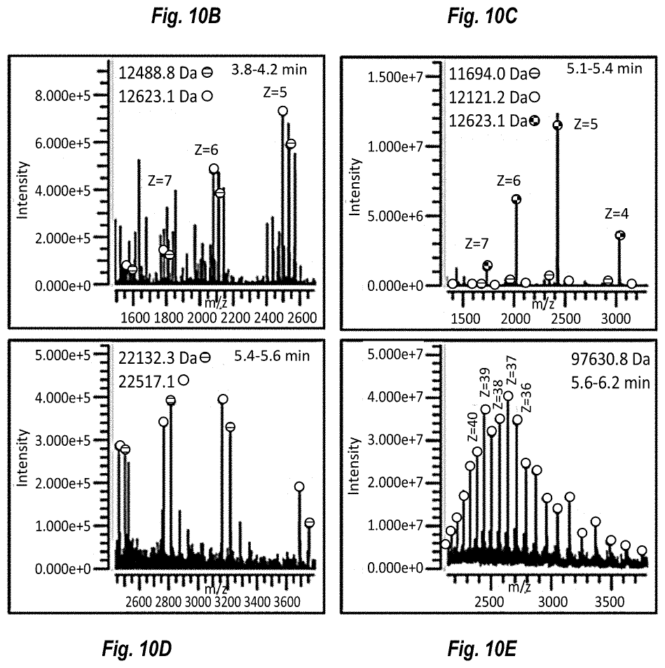

A single method was tested for optimization based on the trends observed across the pH, temperature, and percent organic conditions. A one-pot digestion, with Cathepsin L and D, of trastuzumab was prepared at 37° C. for 18 hours at pH 4. As shown in FIG. 10, 100% digestion efficiency was achieved. The most abundant species in the sample corresponded to the loss of the CH2+CH3 region and generating the F(ab′)2 (≈98 kDa). Assignments were made within 2 Da for species <50 kDa and within 4 Da for species <100 kDa, corresponding to an average error of 31.8 ppm. The peptides observed appeared across all digestion replicates within 1 Da (N=6). The most abundant smaller 1\4W peptides observed mapped to the VH1, VL, and Cl regions (Table 10).

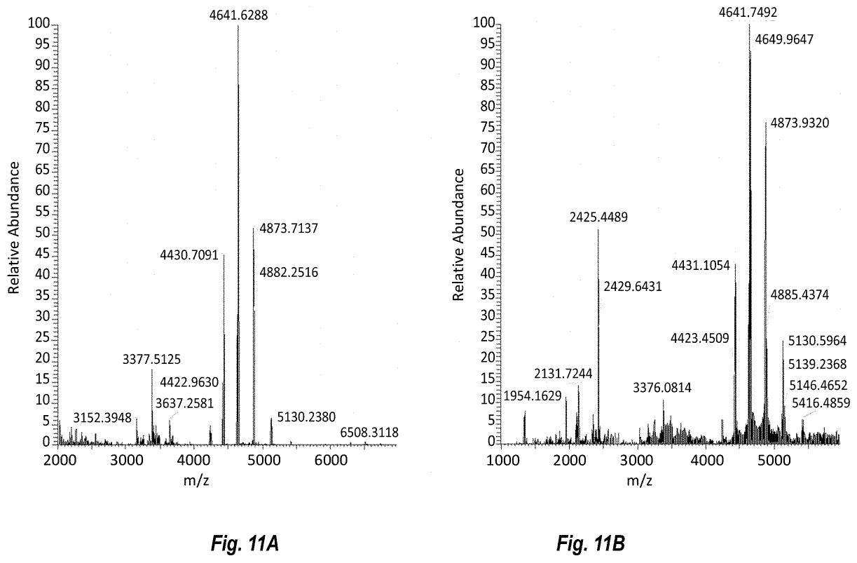

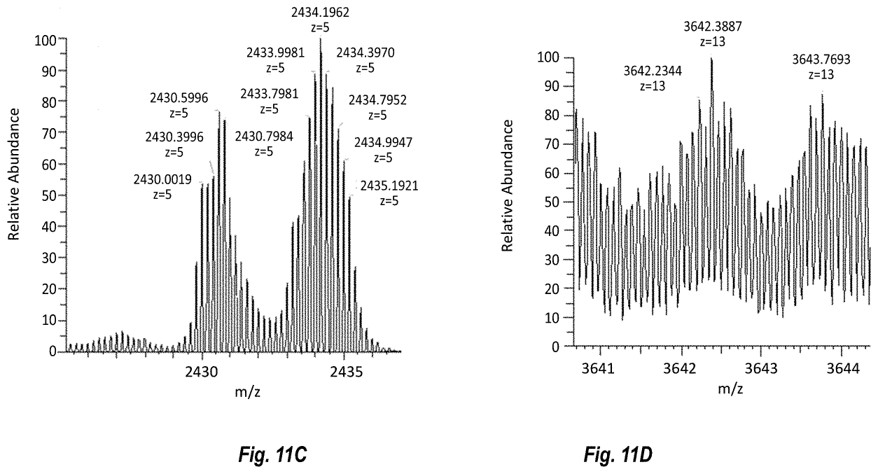

Interestingly, the 47 kDa species characterized in the control studies (FIG. 7B) was not observed in the LC-MS analysis of the optimized digestion, although it was observed later during the nESI infusion for top-down analysis (FIG. 11D). As the F(ab′)2 has a wide elution width (−1 min) and shows fronting, it is possible the F(ab′) co-elutes.

Highly specific cleavage sites were observed across the protein by LC-MS. This resulted in nine polypeptides, including the 98 kDa species, comprising ≈70% of the summed intensities taken over all deconvolved LC peaks (assigned+unassigned). The remainder of the protein fragments observed, but unassigned, largely belonged to small molecular weight species that were 4-5 kDa. As the larger polypeptides provided 100% coverage of the antibody, assignments of the 4-5 kDa species were not attempted, though a complete list is provided in Table 11. Furthermore, at smaller molecular weight, the number of possible sequence assignments increases significantly, due to the consideration of internal digest products, and maintaining the integrity of the identified sites was important.

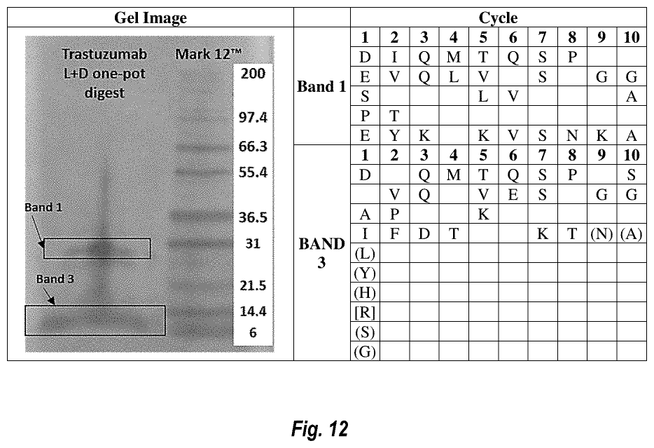

To confirm the specificity of the nine peptide identifications made, Edman degradation was performed. Sequences were confirmed clearly for 7/9 protein fragments identified (FIG. 12). A motif starting with “PT” and “APxxK” was observed, corresponding to the cross-linked masses that result in a species at 22,517.13 Da. While the low abundance and overlapping gel bands precluded the confirmation of the 12,488.77 mass, it was observed that at least a protein fragment starting with a Gly was identified at the expected molecular weight.

### A.3. Top-Down Analysis of the Clipped Protein Fragments

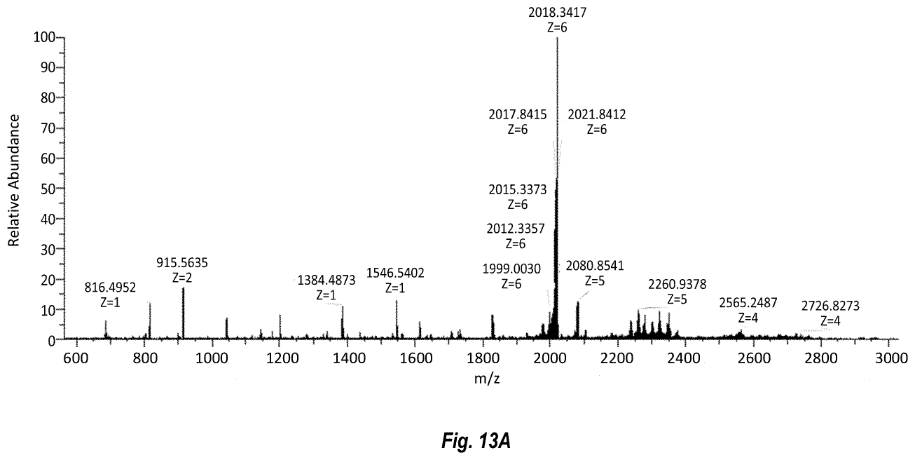

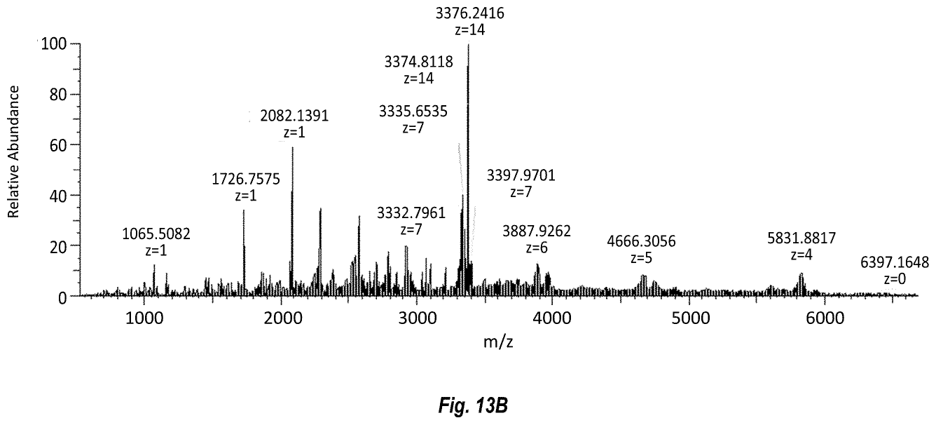

Top down analysis was performed to demonstrate the suitability of the polypeptide size and structure to fragmentation and to provide further validation of the annotated trastuzumab protein fragments (Table 10). To enhance the signal-to-noise of the MS2 spectra, the optimized trastuzumab sample was infused by nESI, versus LC, on the Q Exactive UHMR and precursors were averaged for at least 100 scans (FIG. 11). Product ions were isotopically resolved at a setting of 200,000 resolving power (FIG. 13).

Product ion assignments were made by extracting monoisotopic masses by the Xtract algorithm in Freestyle 1.6 (Thermo Fisher Scientific, Bremen, Germany) and matching them within 10 ppm to their respected predicted sequence (Table 10) in ProSight Lite [62], where cysteines were considered with an H-loss to account for the presence of the disulfide bonds. Subsequently, an in-house database accounting for y-ion NH3 losses and water losses was built and matched to the remaining extracted masses (Table 12). As shown in FIG. 14A, good sequence coverage was obtained on the N- and C-terminus of the 12,121.5 Da precursor, with the fragmentation efficiency reduced between the disulfide bonds. Very large product ions were preserved (Y104 and B109), in order to validate the sequence assignment. Overall, a 29.4% sequence coverage was obtained.

For the 97,630 Da species, assignments were complicated by the large number of disulfide bonds in the subunit. While the inter-chain and/or intra chain could have been reduced to improve coverage, it was important to validate the polypeptide in its bound form to prove the presence of the asymmetric HC cleavage (pairing of a PAPELLG.G and PAPELLGG.PSV). As shown in FIG. 14B, a significant number of y ions corresponded specifically to each HC form. After assignment of the standard y, b, ammonia and water loss ions, the presence of cross-linked species from the inter-linked chains was assessed (Table 13). For the LC, modifications built from the HC sequence NVNHKPSNTKVDKKVEPKSCDKTHT were considered, where C24 was included in every sub-sequence as the site of cross-linking. The N and C-terminal bounds considered represented the start of a HC intra-chain disulfide (C148-C204) or the HC inter-chain disulfide (chain1, C230-chain2, C230), respectively. For the HC, cross-linked modifications were considered from the LC sequence EVTHQGLSSPVTKSFNRGEC, which starts at the amino acid after the last intra-disulfide bonded cysteine and is then cross-linked at the C-terminus. Ions detected from these species confirmed the presence of the asymmetric C-terminus generated from the Cathepsin L/D digest (FIG. 14B). In total, 228 total ions were detected and 33.7% total coverage was obtained, with 26.6% coverage of the LC and 40% coverage of the HC.

### A.4 Application of Optimized Cathepsin L and D Protocol to a Bispecific Antibody

With the optimized protocol established, the application of Cathepsin was further tested on a bispecific IgG1 antibody (anti-Her²/anti-CD3). While bispecific antibodies have identical disulfide bond structures to IgG1, they exhibit significant structural differences. By mutating various amino acids in the Fc region, a “knob and hole” structure is created, whereby the xHer2 and xCD3 chains are potentiated, generating a hetero-dimer [63]. Primary structure changes drive this process, with multiple mutations found between amino acids 26-110 (variable region, CD3 binding), in locals 371 and 408 (knob/hole) and at 297 (preventing glycosylation). Additionally, working with a freshly expressed, research antibody, introduced a new source of variability—the presence of 4% dimer—that is not found in most clinically used drug products.

The Cathepsin proteases behaved as expected throughout the F(ab′) (Table 4). In both the xHer2 and xCD3 LC and HC, cleavages were observed directly after the CDR3 and outside of the disulfide bond region. The LC and CH1 constant regions were homologous, and a single protein fragment was assigned as the digest product for both chains. Cleavage after the hinge yielded five protein fragments, with asymmetric cleavage occurring along the highly favored and conserved GGPSVFLFPPK sequence. Interestingly, no other cleavages were observed within the xCD3 CH2 or CH3 regions. In the anti-Her2 (knob) chain, digestion occurred after the hinge to yield the F(ab′)2 (AA 1-239) and a few amino acids later to yield a CH2+CH3 fragment (AA 261-436). Multiple protein fragments in the CH3 were generated in the knob chain as well. However, the only Fc region cleaved on the xCD3 strand occurred concurrently across both chains. Considering that the Cathepsin proteases are fairly promiscuous, the most likely explanation for this difference is the specific tertiary structure of the bispecific antibody. It is possible that the hole shape or the rigidity of the structure, compared to the knob, makes it difficult for the Cathepsins to “act” in this area, until it is at least partially exposed through cleavage of the xHer2 chain. Validation of this specific knob/hole effect would require testing of a large number of bispecifics, which is outside of the scope of this study. These structural implications may also affect the digestion efficiency. Whereas the optimized protocol resulted in 100% digestion of trastuzumab, 20% of the intact bispecific remained (based on deconvolved peak intensity). This may reflect either slower digestion, as a result of the unique structure, or be caused partially by slower digestion of the dimer, which was reduced from four to zero percent in the final digest.

### B. DISCUSSION

Development of middle down approaches that yield ideal-sized polypeptides for MS sequencing is critical to advancing workflows in for top-down antibody sequencing. The Cathepsin L and D protease offer an efficient, commercially available, inexpensive one-pot digestion to directly enable this workflow.

Under all digestion conditions, peptides giving coverage of the VL, VH, CL, CHL, CH1, CH2, CH3, CL+CH1 (bonded), and F(AB′) were individually observed. The combination of Edman Degradation (N-terminal confirmation), intact mass (<40 ppm matching), and top-down data unambiguously succeeded in sequencing the main cleavage products yielded from the digest, comprising 70% of the total protein signal in the optimized sample. The VL and VH regions were produced in high abundance, with cleavage occurring directly after the CDR3. This region represents the most challenging section to sequence due to its high variability over a short region. The ability to directly sequence a long-read that is the ideal size for top-down sequencing and encompasses the CDR1, CDR2, and CDR3 is a key feature of the middle-down approach presented.

Compared to the most widely employed IdeS protocol, both treatments can directly generate the F(ab′) and the cleavage site at the hinge is nearly identical between the two protocols. Reducing reagents may be used in an IdeS protocol to generate free LC or HC, but these pose size-related challenges for complete top-down sequencing by CID or HCD, whereas in the Cathepsin protocol, the generation of protein fragments offers a direct approach. When combined with existing de novo bottom-up techniques, the generation of protein fragments allows for the masses of sequences to be checked regionally on the Ab, versus against the entire intact Ab. This offers the opportunity to find and localize problematic assignments quickly. As computational tools develop to top-down de novo sequence, the cathepsin approach solves much of the sequencing alignment challenges with the digest remaining in the size range to yield high quality product ion spectra.

HCD is known to lead to limited sequence coverage of Abs across disulfide bonds [20]. Alternative fragmentation techniques, such as electron capture dissociation (ECD) could solve this sequencing challenge and enable direct analysis of the digestion mixture, but are not found on many mass spectrometers. Top-down HCD can achieve 100% coverage of smaller molecular weight species when disulfide bonds are not present. Thus, were this method to be used for Ab de novo sequencing, versus sequence validation as demonstrated in this paper, fractionation of the sample either by chromatography or molecular weight cut-off filters, followed by treatment and clean-up of guanidine hydrochloric acid, would enable in-depth sequence while limiting the number of co-ionized peaks. Additionally, application of guanidine would transition the precursors to higher charge states, which would further improve the fragmentation efficiency.

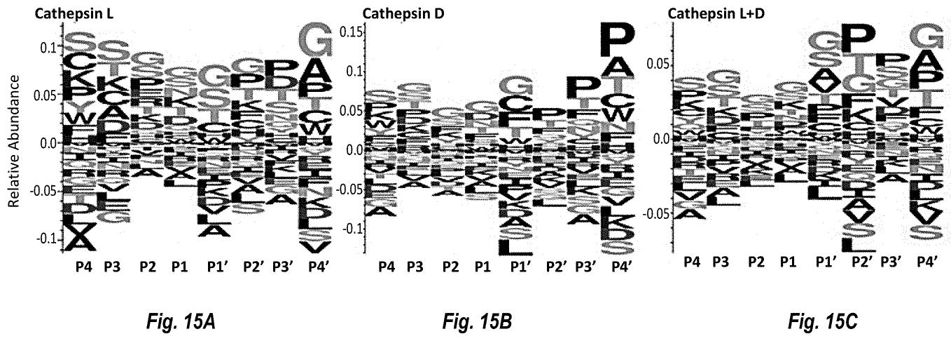

Evaluation of the Cathepsin L and D across Abs showed remarkable consistency for the cleavage sites across all IgG1s, especially when considered in the context of the motif promiscuity. Across all of the tested antibodies (trastuzumab, rituximab, obinutuzumab, eculizumab, and the bispecific antibody), and including the combined L/D digest, 50 unique cleavage sites were identified. Cathepsin L cut at 27 sites and Cathepsin D cut at 28. While there was clear evidence that neutral sites were preferred for cleavage, both Cathepsin proteases otherwise showed little preference for particular amino acids at the p4 to p4′ (FIG. 15). While the literature commonly reports F and R as sites of cleavage by Cathepsin L, and these termini were present, they constituted no more than 10% of the total cleavage products. In comparison to a 2011 study of Cathepsin L activity on HEK293 protein extracts, which found equal enrichment between the 4-6 amino acids enriched per p3-p3′ site, the Abs showed a significantly reduced motif preference, indicating that the higher order versus secondary structure may be most important [48]. This may be why individual studies that have examined Cathepsin L and D protein activity do identify many exceptions to these rules in their reported cleavage sites [47-57]. An alternative possibility is that studies are using different Cathepsin L and D isoforms, which are known to result in different products . Additionally, Cathepsin L and D were shown to have the ability to cleave at a Proline, which is relatively uncommon across all proteases, with the protease EndoPro® as a noticeable exception[64].

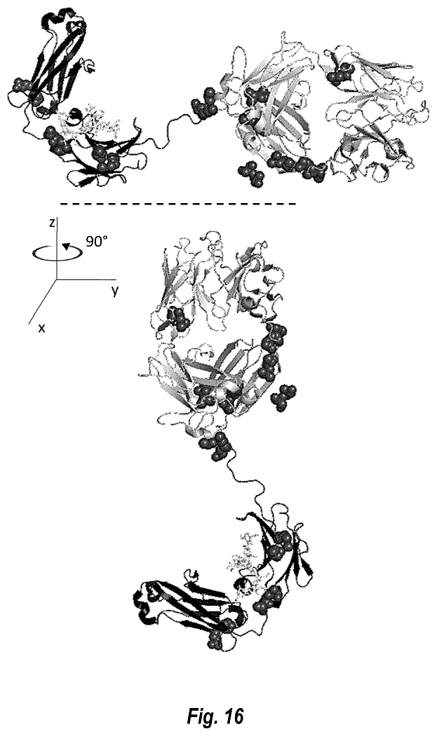

The Cathepsin induced cleavage sites on the Ab′s were compared against its crystal structure to examine a potential role of the secondary and tertiary structure (FIG. 16). No cleavages were observed within any alpha helices, and the vast majority were located in the random coils. The remainder of the cleavage sites were observed within 1-2 amino acids distance from the end of a random coil, but within the start of a beta sheet. The nature of these sites strongly suggests that Cathepsin L and D have limited ability to cleave within the ordered regions of Abs yet may bind to almost any amino acid motif in a flexible and disordered region. The cleavage sites are further constrained by the disulfide bonding patterns. Disulfide bonds provide significant constraints on the final tertiary structure and compared to unbound regions, show considerably less flexibility [65]. While Cathepsin enzymes may be promiscuous at the local amino acid level, it is possible that local flexibility, found outside of disulfide bonded regions, is required to situate the to-be-cleaved antibody sequence inside of the enzymatic pocket. Interestingly, this conclusion is supported by the digestion efficiency comparison carried out under different pH, thermal, and organic conditions. While different efficiencies were observed, the sites cleaved were found to be consistent across the conditions. Despite denaturation that may occur on the secondary structure level, each treatment left the disulfide bonding pattern unaffected, showing that this constraint is the primary factor in determining cleavage location. When digestion was extended to a bispecific antibody, CH2/CH3 digestion in the anti-CD3 arm was prevented beyond the hinge region, contrary to the standard IgG1 Cathepsin L+D digestion pattern found across the anti-Her2 arm. The anti-Her2 cleavages occurred at places with homologous primary sequence and secondary structure to the anti-CD3 strand, suggesting a role for the local flexibility of sequences, domain orientation, or other tertiary (hole) effects in determining Cathepsin L and D digestion.

A comparison of the protein fragments identified in the trastuzumab using standard conditions (pH 7, 37° C., 2 days, 1:20 ratio) versus in the optimized conditions showed interesting differences in their relative abundances (Tables 8-10). For example, the most abundant low molecular weight polypeptide at pH 7 is the 11,197 Da (HC: 1-102 (YYCSRWGGD.F)) fragments, whereas at the optimal pH 4 the most abundant fragment below 20 kDa is the 12,121 Da (LC: 1-110 (RTVA.A)) product. One possibility is that when combined in a single pool, Cathepsin L and D affect each other's activity, either by clipping the other enzyme or via stoichiometry effects when bound to the Ab. Neither the intact mass of Cathepsin L or D was observed during deconvolution; however, some of the observed masses ≈25 kDa could correspond to small clips of Cathepsin L (30 kDa) or a highly clipped form of the ≈45 kDa Cathepsin D. This may account for some of the unidentified species deconvolved in the clipping evaluation.

The clipping patterns observed when using Cathepsins L and D suggest that Cathepsin treatment may also be used as a relatively simple assay to check the disulfide linkages in Abs. Both Cathepsins are likely to produce “single-arm” fragments around 48 kDa for IgG1s, but they do not produce these fragments for the IgG2-B Abs that contain a distinct disulfide bonding pattern. The IgG2-B pattern is difficult to check by non-reduced peptide mapping, and is most commonly performed by Lys-C [66, 67].

Here we used in parallel high resolution native MS and denatured LC-MS on directly infused non-reduced Abs. This approach has the advantages of simplicity, minimum sample preparation, and mild source conditions with little or no in-source fragmentation. Disulfide bond reduction and de-glycosylation can improve sensitivity and simplify the mass spectra and data analysis, but add extra steps to the presented method. The use of software with the capacity to automate intact assignments helped speed the evaluation of new proteases and holds promise as a computational resource to assess natural clipping within cells or antibody by-products. This method generates ideal-sized protein fragments for sequencing, achieves 100% coverage of the Ab distributed across a limited number of protein fragments (nine species), and uses commercially available enzymes, making this workflow suitable as a robust and reproducible middle-down sequencing workflow.

### C. METHODS

### C.1. Chemicals and Materials

The therapeutic Abs, rituximab (MabThera®), obinutuzumab (Gazyva®), and eculizumab (Soliris®) were gifts from Genmab (The Netherlands), while trastuzumab (Herceptin®) and anti-Her²/ₐnti-CD3 bispecific were supplied in-house at Genentech, Inc. Excluding the bispecific, all Ab samples were from expired batches. Prior to use, Ab integrity was checked by native MS to ensure against any post-translational modifications or structure changes (checked by charge state distribution) and the Abs were expected to be of high integrity. All amino acid sequences searched lacked the N-terminal signal peptides and are provided in Table 14. Dithiothreitol (DTT), iodoacetamide (IAA), ammonium acetate (AMAC), acetic acid, formic acid (FA), 8M tris(hydroxymethyl)aminomethane (Tris), methanol (MeOH), and Cathepsins L and D were purchased from Sigma-Aldrich (St Louis, Mo.); phosphate buffer was from Lonza Group AG (Basel, CH) . Acetonitrile (ACN) was purchased from Biosolve BV (North Brabant, NL) and Fisher Scientific (Hampton, N.H.).

C.2. Antibody digestion by Cathepsin L and Cathepsin D

To evaluate the Ab motif suitable for cleavage by Cathepsin L and D, digestion was performed across all therapeutic Abs (rituximab, obinutuzumab, eculizumab, trastuzumab) at a single control condition. Abs were prepared at 5 μM in Milli-Q water and treated individually with Cathepsin L or D at a 1:200 ratio of enzyme to antibody. Samples were incubated at 37° C. for 2 days at neutral pH. The digested Ab samples were buffer exchanged into 150 mM aqueous AMAC (pH 7.5) by centrifugation using a 10 kDa cut-off filter (Merck Millipore, Burlington, Mass.). The final protein concentration was measured by UV absorbance at 280 nm. The digest was adjusted to 2-3 μM and either used directly for native MS analysis or incubated with 4 units of PNGase F overnight using the standard native digestion protocol [22]. PNGase F treated samples were buffer exchanged a second time into 150 mM AMAC (pH 7.5) prior to native MS measurement.

A single Ab, trastuzumab, at a stock solution of 2 mg/mL was then used to explore the cleavage efficiency of each Cathepsin under different digest conditions. Each Cathepsin was resuspended in water at 1 mg/mL and different pH, MeOH, and temperature conditions were tested in triplicate (Table 15). Digests were prepared in a 1:200 enzyme to protein ratio with a final Ab concentration of 0.2 mg/mL. For pH-adjusted preparations, diluent buffer of 50 mM ammonium acetate at the desired pH was added, and comprised 79% of the solution. For LC-UV-MS analysis, 15 μL was injected onto a 2.1×50 mm MAbPac™ RP HPLC column (80° C.) on an Ultimate 3000 LC coupled to a DAD detector (254 nm) and Exactive™ EMR (Thermo Fisher Scientific, Waltham, Mass.). Flow was set to 300 μL/min, where mobile phase A (MPA) was 99.88% water and 0.1% formic acid and 0.02% trifluoroacetic acid and B (MPB) was 90% ACN, 9.88% water, 0.02% trifluoroacetic acid, and 0.1% formic acid. MPB was increased from 5 to 20% at 1 min, to 65% at 9.5 min, to 90% at 10 min, and held at 90% for 2 min before re-equilibration.

A final one-pot reaction of L+D was evaluated on trastuzumab at a pH of 4.0 and temperature of 37° C. for 18 hours. LC-UV-MS was used to evaluate intact masses as described above. Top-down analysis on a Q Exactive™ UHMR was performed on the sample directly buffer exchanged into 50 mM ammonium acetate using a Micro Bio-Spin column according to the manufacturer's directions. The settings were tailored as described in the Static nESI MS section below.

C.3. Static nESI MS of Cathepsin L and D fragments

Samples were analyzed on a modified Exactive™Plus Orbitrap instrument with extended mass range (EMR) (Thermo Fisher Scientific) [68] or a Q Exactive™UHMR [69] using an m/z range of either 500-12,000. The voltage offsets on the transport multi-poles and ion lenses were manually tuned to achieve optimal transmission of protein ions at elevated m/z. Nitrogen was used in the higher-energy collisional dissociation (HCD) cell at a gas pressure of 3-7×10⁻¹⁰ bar. MS parameters used: spray voltage 1.2-1.3 V, source temperature 250° C., source fragmentation and collision energy were varied from 50-80 V, and resolution (at m/z 200) 35,000 or 70,000 for all Abs. The instrument was mass calibrated as described previously using a solution of CsI [22].

### C.3. Edman Degradation

Cathepsin cleaved antibodies from the one-pot trastuzumab L+D digest were separated on a 4-20% Novex™ Tris-Glycine SDS-PAGE gel (Thermo Fisher Scientific) and electroblotted onto PVDF membrane then visualized with Coomassie Brilliant Blue R-250 stain. Bands of interest were excised and subjected to N-terminal sequence analysis using a 494 Procise™ Sequencer (Applied Biosystems, Foster City, Calif.). The resulting mixture of sequences was analyzed using sequencer associated 610 software (Applied Biosystems, Foster City, Calif.) and manually verified.

### C.4. Data Analysis of Antibody Digestion Products

Intact Mass™ v3.2-424 (October, 2018) was used for charge deconvolution (Protein Metrics, San Carlos, Calif.). For initial deconvolutions the default parameters, which included the m/z range 600-9,000, m range 10,000-160,000, m/z spacing 0.04, m/z smoothing sigma 0.02, 0.2 charge spacing, and m spacing 0.5 and m smoothing sigma 3.0 were used. For subsequent deconvolutions, the parameters were tailored to yield the highest quality results. In all cases, the minimum difference between mass peaks was 15, which results in the peak detector sigma to 5 (one-third of minimum difference between mass peaks). A mass matching tolerance of 4 Da was applied for automatic peak annotation.

Details of the Intact Mass™ program, released commercially in 2019, whose matching algorithm for clipped species has not been described in the scientific literature, is thus summarized here. After charge deconvolution [27, 59], the algorithm picks peaks in the neutral mass spectrum using a “Mexican hat” peak detection filter in decreasing order of intensity and with deprioritization for masses found at the shoulder of crowds of peaks. Settings to specify the peak detector width (standard deviation of the positive part of the filter, default=5 Da), the mass range, maximum number, minimum mass spacing, minimum percentage of base peak, and minimum signal-to-noise ratio of picked peaks may be customized.

Deconvolved and picked peaks were matched against theoretical average-isotope masses computed from inputted amino acid sequences (including multiple chains) and a table of natural isotope abundances. A ¹³C abundance (1.079%), characteristic of biological sources, was specified. Average mass was used to avoid off-by-one-Dalton errors in monoisotopic masses, and to provide uniformity across mass spectra that may contain a mix of isotope-resolved and unresolved masses.

Every peptide bond in either light or heavy chain was considered a potential clip site, rather than restricting cleavage to preferred amino acids. In the matching algorithm, a suffix (a sequence containing the C-terminus but not the N-terminus of a chain) starting with Q is not assumed to start with pyro-Glu, whereas a prefix (a sequence containing the N-terminus but not the C-terminus of a chain) sequence is. C's (cysteines) are by default assumed to be disulfide-bonded, with a single odd-numbered C remaining reduced, and Intact Mass subtracts ˜1 Da without trying to predict the pattern. The algorithm is a simple greedy algorithm [70]: each picked peak is matched to the closest theoretical mass within a set mass tolerance. Except for the special case of identical chains cleaved at the same position, Intact Mass computes prefix or suffix sequences by cleaving only a single chain, and summing it with the other intact second chain. For an intact mAb, the 2 LC and 2 HC were concatenated such that Intact Mass would generate prefixes and suffixes by clipping a single chain and leaving the other three chains intact. The software will also consider two identical chains cut at the same position, for example, the F(ab′)2 fragment produced by an IdeS protease cutting between the G's in CPPCPAPELLG.GPS. It will automatically consider a “half Ab” formed by a single LC+HC for clipping.

All assignments were manually validated and all unassigned species were manually evaluated to determine if the algorithm missed an assignment.

### D. REFERENCES

1. Vandermarliere, E., Stes, E., Gevaert, K. & Martens, L. (2016) Resolution of protein structure by mass spectrometry, Mass Spectrom Rev. 35, 653-665.
2. Rathore, D., Faustino, A., Schiel, J., Pang, E., Boyne, M. & Rogstad, S. (2018) The role of mass spectrometry in the characterization of biologic protein products, Expert Review of Proteomics. 15, 431-449.
3. Muth, T., Hartkopf, F., Vaudel, M. & Renard, B. Y. (2018) A Potential Golden Age to Come—Current Tools, Recent Use Cases, and Future Avenues for De Novo Sequencing in Proteomics, Proteomics. 18, 1700150.
4. Standing, K. G. (2003) Peptide and protein de novo sequencing by mass spectrometry, Curr Opin Struct Biol. 13, 595-601.
5. (1999) De Novo Peptide Sequencing via Tandem Mass Spectrometry, J Comput Biol. 6, 327-342.
6. Ma, B., Zhang, K., Hendrie, C., Liang, C., Li, M., Doherty-Kirby, A. & Lajoie, G. (2003) PEAKS: powerful software for peptide de novo sequencing by tandem mass spectrometry, Rapid Commun Mass Spectrom. 17, 2337-2342.
7. Seidler, J., Zinn, N., Boehm, M. E. & Lehmann, W. D. (2010) De novo sequencing of peptides by MS/MS, Proteomics. 10, 634-649.
8. Liu, P., Zhu, X., Wu, W., Ludwig, R., Song, H., Li, R., Zhou, J., Tao, L. & Leone, A. M. (2019) Subunit mass analysis for monitoring multiple attributes of monoclonal antibodies, Rapid Commun Mass Spectrom. 33, 31-40.
9. Srzentić, K., Nagornov, K. O., Fornelli, L., Lobas, A. A., Ayoub, D., Kozhinov, A. N., Gasilova, N., Menin, L., Beck, A., Gorshkov, M. V., Aizikov, K. & Tsybin, Y. O. (2018) Multiplexed Middle-Down Mass Spectrometry as a Method for Revealing Light and Heavy Chain Connectivity in a Monoclonal Antibody, Anal Chem. 90, 12527-12535.
10. Pandeswari, P. B. & Sabareesh, V. (2019) Middle-down approach: a choice to sequence and characterize proteins/proteomes by mass spectrometry, RSC Advances. 9, 313-344.
11. Tsiatsiani, L. & Heck, A. J. R. (2015) Proteomics beyond trypsin, The FEBS Journal. 282, 2612-2626.
12. Vincents, B., von Pawel-Rammingen, U., Björck, L. & Abrahamson, M. (2004) Enzymatic Characterization of the Streptococcal Endopeptidase, IdeS, Reveals That It Is a Cysteine Protease with Strict Specificity for IgG Cleavage Due to Exosite Binding, Biochemistry. 43, 15540-15549.
13. von Pawel-Rammingen, U., Johansson, B. P. & Björck, L. (2002) IdeS, a novel streptococcal cysteine proteinase with unique specificity for immunoglobulin G, The EMBO journal. 21, 1607-1615.
14. Fornelli, L., Ayoub, D., Aizikov, K., Liu, X., Damoc, E., Pevzner, P. A., Makarov, A., Beck, A. & Tsybin, Y. O. (2017) Top-down analysis of immunoglobulin G isotypes 1 and 2 with electron transfer dissociation on a high-field Orbitrap mass spectrometer, J Proteomics. 159, 67-76.
15. Srzentić, K., Zhurov, K. O., Lobas, A. A., Nikitin, G., Fornelli, L., Gorshkov, M. V. & Tsybin, Y. O. (2018) Chemical-Mediated Digestion: An Alternative Realm for Middle-down Proteomics?, J Proteome Res. 17, 2005-2016.
16. McCool, E. N., Chen, D., Li, W., Liu, Y. & Sun, L. (2019) Capillary zone electrophoresis-tandem mass spectrometry with ultraviolet photodissociation (213 nm) for large-scale top-down proteomics, Analytical Methods. 11, 2855-2861.
17. Cannon, J. R., Cammarata, M. B., Robotham, S. A., Cotham, V. C., Shaw, J. B., Fellers, R. T., Early, B. P., Thomas, P. M., Kelleher, N. L. & Brodbelt, J. S. (2014) Ultraviolet photodissociation for characterization of whole proteins on a chromatographic time scale, Anal Chem. 86, 2185-2192.
18. Shaw, J. B., Malhan, N., Vasil'ev, Y. V., Lopez, N. I., Makarov, A., Beckman, J. S. & Voinov, V. G. (2018) Sequencing Grade Tandem Mass Spectrometry for Top-Down Proteomics Using Hybrid Electron Capture Dissociation Methods in a Benchtop Orbitrap Mass Spectrometer, Anal Chem. 90, 10819-10827.
19. Shaw, J. B., Li, W., Holden, D. D., Zhang, Y., Griep-Raming, J., Fellers, R. T., Early, B. P., Thomas, P. M., Kelleher, N. L. & Brodbelt, J. S. (2013) Complete protein characterization using top-down mass spectrometry and ultraviolet photodissociation, J Am Chem Soc. 135, 12646-12651.
20. Catherman, A. D., Skinner, O. S. & Kelleher, N. L. (2014) Top Down proteomics: facts and perspectives, Biochem Biophys Res Commun. 445, 683-693.
21. Tran, J. C., Zamdborg, L., Ahlf, D. R., Lee, J. E., Catherman, A. D., Durbin, K. R., Tipton, J. D., Vellaichamy, A., Kellie, J. F., Li, M., Wu, C., Sweet, S. M. M., Early, B. P., Siuti, N., LeDuc, R. D., Compton, P. D., Thomas, P. M. & Kelleher, N. L. (2011) Mapping intact protein isoforms in discovery mode using top-down proteomics, Nature. 480, 254-258.
22. Rosati, S., Yang, Y., Barendregt, A. & Heck, A. J. R. (2014) Detailed mass analysis of structural heterogeneity in monoclonal antibodies using native mass spectrometry, Nat Protoc. 9, 967-976.
23. Bailey, A. O., Han, G., Phung, W., Gazis, P., Sutton, J., Josephs, J. L. & Sandoval, W. (2018) Charge variant native mass spectrometry benefits mass precision and dynamic range of monoclonal antibody intact mass analysis, Mabs-Austin. 10, 1214-1225.
24. Phung, W., Han, G., Polderdijk, S. G. I., Dillon, M., Shatz, W., Liu, P., Wei, B., Suresh, P., Fischer, D., Spiess, C., Bailey, A., Carter, P. J., Lill, J. R. & Sandoval, W. (2019) Characterization of bispecific and mispaired IgGs by native charge-variant mass spectrometry, Int J Mass spectrom. 446, 116229.
25. Čaval, T., Tian, W., Yang, Z., Clausen, H. & Heck, A. J. R. (2018) Direct quality control of glycoengineered erythropoietin variants, Nat Commun. 9, 3342.
26. Wohlschlager, T., Scheffler, K., Forstenlehner, I. C., Skala, W., Senn, S., Damoc, E., Holzmann, J. & Huber, C. G. (2018) Native mass spectrometry combined with enzymatic dissection unravels glycoform heterogeneity of biopharmaceuticals, Nat Commun. 9, 1713.
27. Campuzano, I. D. G., Robinson, J. H., Hui, J. O., Shi, S. D. H., Netirojjanakul, C., Nshanian, M., Egea, P. F., Lippens, J. L., Bagal, D., Loo, J. A. & Bern, M. (2019) Native and Denaturing MS Protein Deconvolution for Biopharma: Monoclonal Antibodies and Antibody—Drug Conjugates to Polydisperse Membrane Proteins and Beyond, Anal Chem. 91, 9472-9480.
28. Marcoux, J., Wang, S. C., Politis, A., Reading, E., Ma, J., Biggin, P. C., Zhou, M., Tao, H., Zhang, Q., Chang, G., Morgner, N. & Robinson, C. V. (2013) Mass spectrometry reveals synergistic effects of nucleotides, lipids, and drugs binding to a multidrug resistance efflux pump, P Natl Acad Sci USA. 110, 9704-9709.
29. Yang, Y., Liu, F., Franc, V., Halim, L. A., Schellekens, H. & Heck, A. J. R. (2016) Hybrid mass spectrometry approaches in glycoprotein analysis and their usage in scoring biosimilarity, Nat Commun. 7, 13397.
30. Greisch, J.-F., Tamara, S., Scheltema, R. A., Maxwell, H. W. R., Fagerlund, R. D., Fineran, P. C., Tetter, S., Hilvert, D. & Heck, A. J. R. (2019) Expanding the mass range for UVPD-based native top-down mass spectrometry, Chemical Science. 10, 7163-7171.
31. Compton, P. D., Zamdborg, L., Thomas, P. M. & Kelleher, N. L. (2011) On the scalability and requirements of whole protein mass spectrometry, Anal Chem. 83, 6868-6874.
32. McLuckey, S. A. & Stephenson, J. L. (1998) Ion/ion chemistry of high-mass multiply charged ions, Mass Spectrom Rev. 17, 369-407.
33. Ge, Y., Lawhorn, B. G., ElNaggar, M., Strauss, E., Park, J.-H., Begley, T. P. & McLafferty, F. W. (2002) Top Down Characterization of Larger Proteins (45 kDa) by Electron Capture Dissociation Mass Spectrometry, J Am Chem Soc. 124, 672-678.
34. Reid, G. E., Wu, J., Chrisman, P. A., Wells, J. M. & McLuckey, S. A. (2001) Charge-State-Dependent Sequence Analysis of Protonated Ubiquitin Ions via Ion Trap Tandem Mass Spectrometry, Anal Chem. 73, 3274-3281.
35. Zhang, H., Cui, W., Wen, J., Blankenship, R. E. & Gross, M. L. (2011) Native Electrospray and Electron-Capture Dissociation FTICR Mass Spectrometry for Top-Down Studies of Protein Assemblies, Anal Chem. 83, 5598-5606.
36. Syka, J. E. P., Coon, J. J., Schroeder, M. J., Shabanowitz, J. & Hunt, D. F. (2004) Peptide and protein sequence analysis by electron transfer dissociation mass spectrometry, P Natl Acad Sci USA. 101, 9528-9533.
37. Moelleken, J., Endesfelder, M., Gassner, C., Lingke, S., Tomaschek, S., Tyshchuk, O., Lorenz, S., Reiff, U. & Mølhøj, M. (2017) GingisKHAN™ protease cleavage allows a high-throughput antibody to Fab conversion enabling direct functional assessment during lead identification of human monoclonal and bispecific IgG1 antibodies, Mabs-Austin. 9, 1076-1087.
38. Zhang, L., English, A. M., Bai, D. L., Ugrin, S. A., Shabanowitz, J., Ross, M. M., Hunt, D. F. & Wang, W.-H. (2016) Analysis of Monoclonal Antibody Sequence and Post-translational Modifications by Time-controlled Proteolysis and Tandem Mass Spectrometry, Molecular & cellular proteomics: MCP. 15, 1479-1488.
39. Mao, Y., Zhang, L., Kleinberg, A., Xia, Q., Daly, T. J. & Li, N. (2019) Fast protein sequencing of monoclonal antibody by real-time digestion on emitter during nanoelectrospray, Mabs-Austin. 11, 767-778.
40. Jones, R. G. A. & Landon, J. (2002) Enhanced pepsin digestion: a novel process for purifying antibody F(ab′)2 fragments in high yield from serum, J Immunol Methods. 263, 57-74.
41. Wu, C., Tran, J. C., Zamdborg, L., Durbin, K. R., Li, M., Ahlf, D. R., Early, B. P., Thomas, P. M., Sweedler, J. V. & Kelleher, N. L. (2012) A protease for ‘middle-down’ proteomics, Nat Methods. 9, 822-824.
42. Srzentić, K., Fornelli, L., Laskay, Ü. A., Monod, M., Beck, A., Ayoub, D. & Tsybin, Y. O. (2014) Advantages of Extended Bottom-Up Proteomics Using Sap9 for Analysis of Monoclonal Antibodies, Anal Chem. 86, 9945-9953.
43. Smith, S. M. & Gottesman, M. M. (1989) Activity and deletion analysis of recombinant human cathepsin L expressed in Escherichia coli, J Biol Chem. 264, 20487-95.
44. Kirschke, H., Kembhavi, A. A., Bohley, P. & Barrett, A. J. (1982) Action of rat liver cathepsin L on collagen and other substrates, The Biochemical journal. 201, 367-372.
45. Press, E. M., Porter, R. R. & Cebra, J. (1960) The isolation and properties of a proteolytic enzyme, cathepsin D, from bovine spleen, The Biochemical journal. 74, 501-514.
46. Brix, K. (2005) Lysosomal Proteases: Revival of the Sleeping Beauty. in Madame Curie Bioscience Database (Saftig, P., ed), Landes Bioscience, Austin (Tex.).
47. Dunn, A. D., Crutchfield, H. E. & Dunn, J. T. (1991) Thyroglobulin processing by thyroidal proteases. Major sites of cleavage by cathepsins B, D, and L, J Biol Chem. 266, 20198-204.
48. Biniossek, M. L., Nägler, D. K., Becker-Pauly, C. & Schilling, O. (2011) Proteomic Identification of Protease Cleavage Sites Characterizes Prime and Non-prime Specificity of Cysteine Cathepsins B, L, and S, J Proteome Res. 10, 5363-5373.
49. Gosalia, D. N., Salisbury, C. M., Ellman, J. A. & Diamond, S. L. (2005) High Throughput Substrate Specificity Profiling of Serine and Cysteine Proteases Using Solution-phase Fluorogenic Peptide Microarrays, Molecular &amp; Cellular Proteomics. 4, 626-636.
50. Puzer, L., Cotrin, S. S., Alves, M. F. M., Egborge, T., Araújo, M. S., Juliano, M. A., Juliano, L., Brömme, D. & Carmona, A. K. (2004) Comparative substrate specificity analysis of recombinant human cathepsin V and cathepsin L, Arch Biochem Biophys. 430, 274-283.
51. Hu, Y., Morioka, K. & Itoh, Y. (2007) Existence of Cathepsin L and its Chracterization in Red Bulleye Surimi., Pakistan Journal of Biological Sciences. 10, 78-83.
52. Luo, H. B., Tie, L., Cao, M. Y., Hunter, A. K., Pabst, T. M., Du, J. L., Field, R., Li, Y. L. & Wang, W. K. (2019) Cathepsin L Causes Proteolytic Cleavage of Chinese-Hamster-Ovary Cell Expressed Proteins During Processing and Storage: Identification, Characterization, and Mitigation, Biotechnol Progr. 35.
53. Tang, J. & Wong, R. N. S. (1987) Evolution in the structure and function of aspartic proteases, J Cell Biochem. 33, 53-63.
54. Sun, H., Lou, X., Shan, Q., Zhang, J., Zhu, X., Zhang, J., Wang, Y., Xie, Y., Xu, N. & Liu, S. (2013) Proteolytic Characteristics of Cathepsin D Related to the Recognition and Cleavage of Its Target Proteins, PLOS ONE. 8, e65733.
55. van Noort, J. M. & van der Drift, A. C. (1989) The selectivity of cathepsin D suggests an involvement of the enzyme in the generation of T-cell epitopes, J Biol Chem. 264, 14159-14164.
56. Woessner Jr., J. (1977) Specificity and biological role of cathepsin D., Adv Exp Med Biol. 95, 313-327.
57. Bee, J. S., Tie, L., Johnson, D., Dimitrova, M. N., Jusino, K. C. & Afdahl, C. D. (2015) Trace levels of the CHO host cell protease cathepsin D caused particle formation in a monoclonal antibody product, Biotechnol Progr. 31, 1360-1369.
58. Zhang, Z., Pan, H. & Chen, X. (2009) Mass spectrometry for structural characterization of therapeutic antibodies, Mass Spectrom Rev. 28, 147-176.
59. Bern, M., Caval, T., Kil, Y. J., Tang, W., Becker, C., Carlson, E., Kletter, D., Sen, K. I., Galy, N., Hagemans, D., Franc, V. & Heck, A. J. R. (2018) Parsimonious Charge Deconvolution for Native Mass Spectrometry, J Proteome Res. 17, 1216-1226.
60. Terra, W. R., Dias, R. O. & Ferreira, C. (2019) Recruited lysosomal enzymes as major digestive enzymes in insects, Biochem Soc Trans. 47, 615-623.
61. Honey, K. & Rudensky, A. Y. (2003) Lysosomal cysteine proteases regulate antigen presentation, Nature Reviews Immunology. 3, 472-482.
62. Fellers, R. T., Greer, J. B., Early, B. P., Yu, X., LeDuc, R. D., Kelleher, N. L. & Thomas, P. M. (2015) ProSight Lite: graphical software to analyze top-down mass spectrometry data, Proteomics. 15, 1235-1238.
63. Merchant, A. M., Zhu, Z., Yuan, J. Q., Goddard, A., Adams, C. W., Presta, L. G. & Carter, P. (1998) An efficient route to human bispecific IgG, Nat Biotechnol. 16, 677-681
64. van der Laarse, S. A. M., van Gelder, C. A. G. H., Bern, M., Akeroyd, M., Olsthoorn, M. M. A. & Heck, A. J. R. Targeting proline in (phospho)proteomics, The FEBS Journal. n/a.
65. Isenman, D. E., Dorrington, K. J. & Painter, R. H. (1975) The Structure and Function of Immunoglobulin Domains, II The Importance of Interchain Disulfide Bonds and the Possible Role of Molecular Flexibility in the Interaction Between Immunoglobulin G and Complement. 114, 1726-1729.
66. Wypych, J., Li, M., Guo, A., Zhang, Z., Martinez, T., Allen, M. J., Fodor, S., Kelner, D. N., Flynn, G. C., Liu, Y. D., Bondarenko, P. V., Ricci, M. S., Dillon, T. M. & Balland, A. (2008) Human IgG2 antibodies display disulfide-mediated structural isoforms, The Journal of biological chemistry. 283, 16194-16205.
67. Resemann, A., Liu-Shin, L., Tremintin, G., Malhotra, A., Fung, A., Wang, F., Ratnaswamy, G. & Suckau, D. (2018) Rapid, automated characterization of disulfide bond scrambling and IgG2 isoform determination, Mabs-Austin. 10, 1200-1213.
68. Rose, R. J., Damoc, E., Denisov, E., Makarov, A. & Heck, A. J. R. (2012) High-sensitivity Orbitrap mass analysis of intact macromolecular assemblies, Nat Methods.
69. van de Waterbeemd, M., Fort, K. L., Boll, D., Reinhardt-Szyba, M., Routh, A., Makarov, A. & Heck, A. J. R. (2017) High-fidelity mass analysis unveils heterogeneity in intact ribosomal particles, Nat Methods. 14, 283-286.
70. Temlyakov, V. (2008) Greedy Approximation in Acta Numerica (Iserles, A., ed) pp. 235-409, Cambridge University Press, Cambridge, England.
71. Thomsen, M. C. F. & Nielsen, M. (2012) Seq2Logo: a method for construction and visualization of amino acid binding motifs and sequence profiles including sequence weighting, pseudo counts and two-sided representation of amino acid enrichment and depletion, Nucleic Acids Res. 40, W281-W287.

### E. TABLES

TABLE 1
Conditions tested for Cathepsin D/L
activity at a 1:20 mAb:enzyme ratio.

| | | | % | Time |
|---|---|---|---|---|
| ID | Temp | pH | MeOH | (Days) |
| A | RT | 7 | 0 | 2 |
| B | 37 | 3 | 0 | 2 |
| C | 50 | 3 | 0 | 2 |
| D | 37 | 5 | 0 | 2 |
| E | 50 | 5 | 0 | 2 |
| F | 37 | 7 | 10 | 2 |
| G | 37 | 7 | 30 | 2 |
| H | 37 | 7 | 50 | 2 |
| I | 37 | 5 | 50 | 2 |
| J | RT | 7 | 0 | 7 |

TABLE 2
Rituximab exposed to Cathepsin L.^{a}

| | Expected | | |
|---|---|---|---|
| Mass | mass | Intensity | Assignment |
| 9474.13 | 9475.49 | 6.90E+05 | LC: 1-89 (...AATYYCQQ.W) |
| 9963.45 | 9963.99 | 3.25E+06 | LC: 1-93 (...YCQQWTSN.P) |
| 46695.91 | 46696.78 | 1.53E+06 | LC + HC: 1-224 (...KKAEPKSC.D) |
| 46812.32 | 46811.87 | 1.19E+06 | LC + HC: 1-225 (...KAEPKSCD.K) |
| 46939.33 | 46940.04 | 1.81E+06 | LC + HC: 1-226 (...AEPKSCDK.T) |
| 46961.08 | | 8.77E+05 | ND |
| 47041.12 | 47041.14 | 7.47E+05 | LC + HC: 1-227 (...EPKSCDKT.H) |
| 47177.9 | 47178.28 | 4.48E+06 | LC + HC: 1-228 (...PKSCDKTH.T) |
| 47200.18 | | 2.01E+06 | ND |
| 47218.67 | | 1.12E+06 | ND |
| 47238.72 | | 7.64E+05 | ND |
| 47278.34 | 47279.39 | 9.37E+05 | LC + HC: 1-229 (...KSCDKTHT.C) |

^{a}The cleavage site is indicated by the AA sequence numbers and the Assignments that were not made are indicated by “ND”.

TABLE 3
Rituximab exposed to Cathepsin D.^{a}

| | Expected | | |
|---|---|---|---|
| Mass | mass | Intensity | Assignment |
| 9473.64 | 9475.49 | 6.38E+06 | LC: 1-89 (...AATYYCQQ.W) |
| 9963.86 | 9963.99 | 1.77E+07 | LC: 1-93 (...YCQQWTSN.P) |
| 46696.39 | 46696.78 | 6.27E+06 | LC + HC: 1-224 (...KKAEPKSC.D) |
| 46809.48 | 46811.87 | 3.52E+06 | LC + HC: 1-225 (...KAEPKSCD.K) |
| 46938.04 | 46940.04 | 7.13E+06 | LC + HC: 1-226 (...AEPKSCDK.T) |
| 46971.12 | | 1.66E+06 | ND |
| 47040.95 | 47041.14 | 2.35E+06 | LC + HC: 1-227 (...EPKSCDKT.H) |
| 47177.29 | 47178.28 | 1.51E+07 | LC + HC: 1-228 (...PKSCDKTH.T) |
| 47210.9 | | 1.85E+06 | ND |
| 47277.65 | 47279.39 | 2.20E+06 | LC + HC: 1-229 (...KSCDKTHT.C) |
| 97538.10 | 97539.97 | 2.92E+06 | 2LC + HC: 1-245 (...PELLGGPSVF.L) + HC: |
| | | | 1-244 (...PELLGGPSV.F) |
| 97684.24 | 97687.14 | 4.87E+06 | 2LC + 2HC: 1-245 (...PELLGGPSVF.L) |

^{a}The cleavage site is indicated by the AA sequence numbers and the Assignments that were not made are indicated by a “ND”.

TABLE 4
Protein fragments from the Cathepsin L and D digestion of the bispecific IgGl
cathepsin. Where the homology of the anti-Her2 and anti-CD3 chains aligned perfectly,
protein fragments are listed in the “non-specific” table section. For fragments
corresponding to a unique sequence, the relevant chain is listed.
Anti Her2 (Knob)

| | Expected | | |
|---|---|---|---|
| Mass | Mass | Intensity | Assignment |
| 9134.1 | 9134.2 | 9.67E+03 | HC: 22-102 (SLRLS.C...SRWGGD.G) |
| 9375.2 | 9375.5 | 1.07E+03 | HC: 19-101 (PGGSL.R...SWRGG.D) |
| 11165.6 | 11165.5 | 8.95E+03 | HC: 101-209 (SRWG.G...NVNHKP.S) |
| 11718.8 | 11719.1 | 8.60E+03 | HC: 12-116 (SGGGL.V...QGTLV.T) |
| 11779.1 | 11779.1 | 3.04E+04 | HC: 14-199 (GGLVQ.P...LVTVS.S) |
| 12392.1 | 12391.9 | 3.28E+05 | HC: 101-220 (SRWG.G...KKVEP.K) |
| 12821.0 | 12821.4 | 3.25E+04 | HC: 337-449 (PAPIE.K...SLSPG.) |
| 13442.4 | 13442.1 | 1.62E+04 | HC: 331-449 (VSNKA.L...SLSPG.) |
| 9184.0 | 9184.2 | 1.62E+03 | HC: 364-443 (REEMTK.N...HYTQKS.L) |
| 13513.5 | 13513.2 | 1.45E+04 | HC: 330-449 (KVSNK.A...SLSPG.) |
| 13755.5 | 13755.5 | 5.73E+04 | HC: 328-449 (EYKCKVS.N...SLSPG.) |
| 20091.1 | 20090.6 | 7.61E+04 | HC: 261-436 (SRTP.E...HEALH.N) |
| 12820.5 | 12820.3 | 6.61E+05 | LC: 7-124 (QMTQ.S...PSDEQ.L) |
| 20039.5 | 20041.3 | 1.22E+04 | LC: 24-205 (VTIT.C...LSSP.V) |
| 11534.0 | 11539.9 | 8.51E+03 | LC: 6-111 (DIQMT.Q...KRTVA.A) |
| 23437.8 | 23438.0 | 3.83E+04 | LC:1-214 |

Anti-CD3 (Hole)

| | Expected | | |
|---|---|---|---|
| Mass | Mass | Intensity | Assignment |
| 11804.5 | 11804.1 | 3.67E+04 | HC: 22-126 (SLRLS.C...SSASTK.G) |
| 9323.9 | 9324.3 | 1.23E+03 | LC: 20-102 (GDRV.T...GQGT.K) |
| 12811.7 | 12811.3 | 6.92E+03 | LC: 1-116 (...APSVF.I) |
| 23626.5 | 23626.2 | 3.46E+04 | LC: 1-214 |

Her2/CD3 non-specific

| | Expected | | |
|---|---|---|---|
| Mass | Mass | Intensity | Assignment |
| 9077.5 | 9077.1 | 6.36E+03 | HC: 118-207 (GTLVT.V...CNVNH.K) |
| 7198.0 | 7198.1 | 1.30E+04 | HC: 142-211 (SKSTS.G...NVNHKP.S) |
| 8903.3 | 8903.8 | 4.11E+03 | LC: 117-196 (PSVF.L...VYACEV.T) |
| 47063.6 | 47064.3 | 8.78E+3 | Her2 LC + CD3 LC |
| 98646.0 | 98650.4 | 5.97E+06 | Her2 LC + CD3 LC + HC: 1-240 (...PAPELLGG.P) + HC 1- |
| | | | 244 (...PAPELLGGPSVF.L) |
| 99069.7 | 99071.0 | 3.18E+06 | Her2 LC + CD3 LC + HC: 1-242 (...ELLGGPS.V) + HC 1- |
| | | | 246 (...GGPSVFLF.P) |
| 99262.4 | 99265.2 | 3.08E+06 | Her2 LC + CD3 LC + HC: 1-242 (...ELLGGPS.V) + HC 1- |
| | | | 248 (...GGPSVFLFPP.K) |
| 98924.0 | 98926.8 | 7.55E+06 | Her2 LC + CD3 LC +HC: 1-239 (...PAPELLG.G) + HC 1- |
| | | | 247(...LGGPSVFLFP.P) |
| 99148.4 | 99152.1 | 4.90E+06 | Her2 LC + CD3 LC +HC: 1-239 (...PAPELLG.G) + HC 1- |
| | | | 249 (...LGGPSVFLFPPK.P) |

TABLE 5a
Obinutuzumab Cathepsin L digest assignments. Obinutuzumab exposed to
Cathepsin L gives one clip in the CDR H3, clips at the end of the
variable part of the HC, and a complete sequence of clips in the heavy
chain hinge region. All heavy chain sequences of obinutuzumab begin with
N-terminal pyro-Glu rather than Glu.

| | Expected | | |
|---|---|---|---|
| Mass | mass | Intensity | Assignment |
| 10866.33 | 10867.06 | 8.80E+04 | HC 1-99 (...AVYYCARN.V) |
| 14681.96 | 14682.36 | 4.55E+04 | HC 1-135 (...FPLAPSSK.S) |
| 14869.87 | 14870.55 | 3.02E+04 | HC 1-137 (...LAPSSKST.S) |
| 14957.54 | 14957.62 | 2.73E+04 | HC 1-138 (...APSSKSTS.G) |
| 15014.36 | 15014.68 | 6.57E+04 | HC 1-139 (...PSSKSTSG.G) |
| 22544.58 | 22545.16 | 2.27E+04 | LC 1-206 (...CEVTHQGL.S) |
| 23491.56 | 23492.22 | 2.59E+04 | HC 1-220 (...VDKKVEPK.S) |
| 23834.75 | 23835.55 | 9.00E+04 | LC 1-218 (...TKSFNRGE.C) |
| 23904.34 | | 4.75E+04 | ND |
| 23938.56 | 23938.69 | 3.96E+04 | LC |
| 24057.36 | 23957.83 | 2.82E+05 | LC + Cysteinylation |
| 24243.96 | | 4.77E+04 | ND |
| 47618.71 | 47619.12 | 1.81E+06 | LC + HC: 1-222 (...KKVEPKSC.D) |
| 47734.36 | 47734.21 | 8.32E+05 | LC + HC: 1-223 (...KVEPKSCD.K) |
| 47861.76 | 47862.38 | 1.92E+06 | LC + HC: 1-224 (...VEPKSCDK.T) |
| 47962.73 | 47963.49 | 7.09E+05 | LC + HC: 1-225 (...EPKSCDKT.H) |
| 48099.86 | 48100.63 | 5.31E+06 | LC + HC: 1-226 (...PKSCDKTH.T) |
| 48201.09 | 48201.73 | 1.00E+06 | LC + HC: 1-227 (...KSCDKTHT.C) |

TABLE 5b
Obinutuzumab Cathepsin D digest assignments. Obinutuzumab exposed to
Cathepsin D gives a sequence of clips in the heavy chain hinge region, along
with cuts of the heavy chain separating F(ab’)2 from Fc.

| | Expected | | |
|---|---|---|---|
| Mass | mass | Intensity | Assignment |
| 47619.25 | 47619.12 | 3.48E+05 | LC + HC: 1-222 (...KKVEPKSC.D) |
| 47734.72 | 47734.21 | 2.40E+05 | LC + HC: 1-223 (...KVEPKSCD.K) |
| 47861.84 | 47862.38 | 4.35E+05 | LC + HC: 1-224 (...VEPKSCDK.T) |
| 48101.69 | 48100.63 | 8.39E+05 | LC + HC: 1-226 (...PKSCDKTH.T) |
| 49616.22 | 49618.4 | 4.10E+05 | LC + HC: 1-242 (...ELLGGPSV.F) |
| 49765.03 | 49765.58 | 7.27E+05 | LC + HC: 1-243 (...LLGGPSVF.L) |
| 99381.38 | | 5.71E+06 | 2LC + HC: 1-245 (...ELLGGPSVF.L) + HC: 1- |
| | | | 244 (...PELLGGPSV.F) |
| 99528.16 | | 1.57E+07 | 2LC + 2HC: 1-245 (...ELLGGPSVF.L) |
| 99642.25 | | 2.25E+06 | 2LC + HC: 1-245 (...ELLGGPSVF.L) + HC: 1- |
| | | | 246 (...LLGGPSVFL.F) |

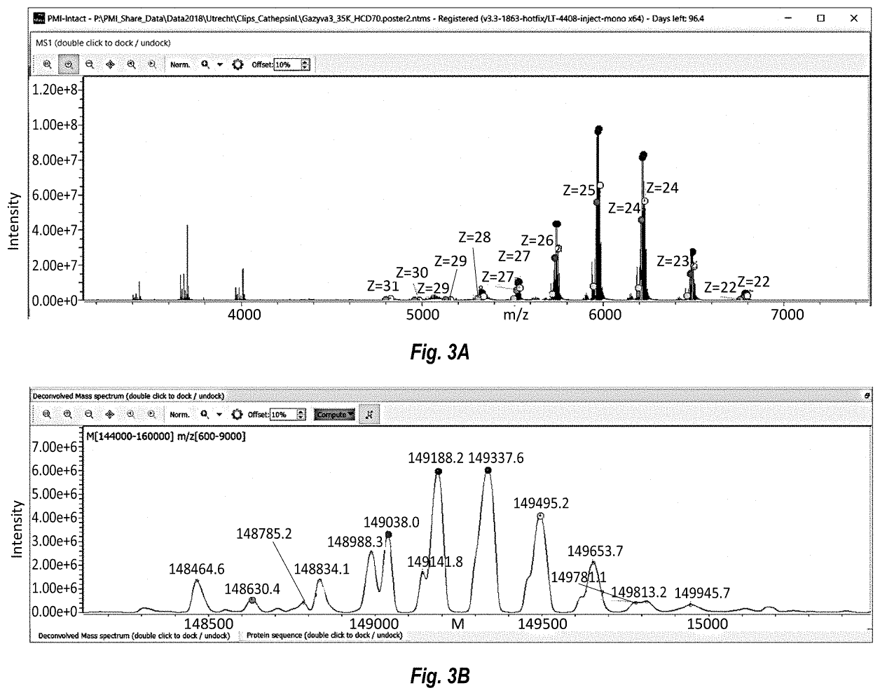

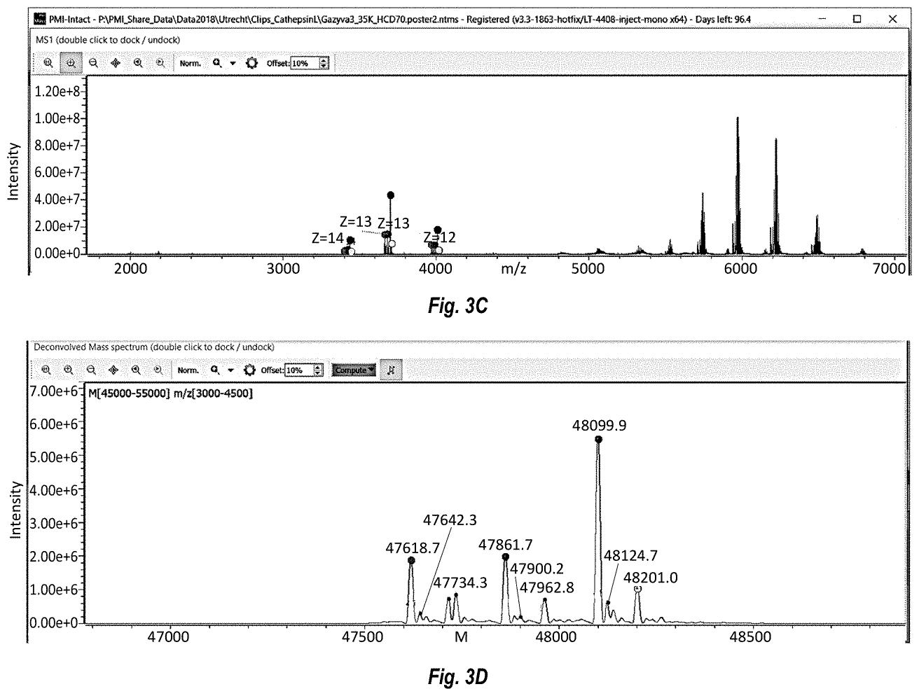

TABLE 6
Eculizumab Cathepsin L digest assignments. Eculizumab exposed to
Cathepsin L gives clips in CDR H3. Unlike the IgG1 mAbs, eculizumab
does not show heavy chain clips that cut off a single antibody arm,
evidence that eculizumab has the IgG2-B disulfide bonding pattern
shown in FIG 3.

| | Expected | | |
|---|---|---|---|
| Mass | mass | Intensity | Assignment |
| 11437.82 | 11438.74 | 1.28E+05 | HC: 1-102 (...YCARYFFG.S) |
| 11524.84 | 11525.82 | 1.10E+05 | HC: 1-103 (...CARYFFGS.S) |
| 11823.08 | 11824.12 | 4.96E+05 | HC: 1-106 (...YFFGSSPN.W) |
| 11838.69 | | 2.93E+05 | ND |
| 11854.45 | | 1.38E+05 | ND |

TABLE 7
Eculizumab Cathepsin D digest assignments. Cathepsin D clips eculizumab
in CDR H3 and also cuts F(ab’)2 from Fc.

| Mass | Expected mass | Intensity | Assignment |
|---|---|---|---|
| 11233.7 | 11234.51 | 4.14E+04 | HC: 1-100 (...VYYCARYF.F) |
| 11396.2 | | 1.39E+05 | ND |
| 11438.24 | 11438.74 | 3.58E+05 | HC: 1-102 (...YCARYFFG.S) |
| 11525.41 | 11525.82 | 3.07E+05 | HC: 1-103 (...CARYFFGS.S) |
| 11806.3 | 11806.68 | 1.50E+05 | ND |
| 11823.14 | 11824.12 | 1.65E+06 | HC: 1-106 (...YFFGSSPN.W) |
| 11838.81 | | 6.61E+05 | ND |
| 98303.1 | 98302.14 | 1.04E+06 | 2LC + HC: 1-241 |
| | | | (...PVAGPSV.F) + HC: 1-242 |
| | | | (...VAGPSVF.L) |
| 98447.76 | 98449.31 | 4.75E+06 | 2LC + 2HC: 1-242 |
| | | | (...PVAGPSVF.L) |
| 98482.05 | 98479.18 | 8.02E+05 | ND |
| 98562.68 | 98562.47 | 1.27E+06 | 2LC + HC: 1-242 |
| | | | (...VAGPSVF.L) + HC: 1-243 |
| | | | (...AGPSVFL.F) |
| 98609.32 | 98608.4 | 6.68E+05 | ND |
| 99586.95 | 99585.25 | 8.48E+05 | ND |

TABLE 8
Trastuzumab Cathepsin L digest assignments for the pH 7, 37° C., 2 days,
1:20 sample and analyzed by LC-MS on a QExactive ™ EMR.

| | Expected | | |
|---|---|---|---|
| Mass | mass | Intensity | Assignment |
| 23439.23 | 23439.06 | 5.10E+04 | LC: 1-214 |
| 23557.22 | ND | 2.66E+04 | |
| 23405.07 | ND | 1.98E+04 | |
| 46877.3 | ND | 1.81E+04 | |
| 23485.72 | ND | 1.67E+04 | |
| 23487.11 | ND | 1.25E+04 | |
| 23744.49 | ND | 1.08E+04 | |
| 23520.05 | ND | 9.75E+03 | |
| 23336.27 | 23335.92 | 8.77E+03 | LC: 1-213 (...SSPVTKSFNRGE.C) |
| 15051.23 | 15050.77 | 8.23E+03 | HC: 1-140 (...PSSKSTSG.G) |
| 20092.3 | 20093.39 | 6.78E+03 | LC: 9-196 (S.SLSASV...HKVYACEV.T) |
| 23580.69 | 23581.22 | 4.88E+03 | HC: 100-223 (W.GGDGFY...KKVEPKSC.D) |
| | | | LC: 117-214 (F.IFPPSDEQ...FNRGEC) |
| 11197.74 | 11198.49 | 1.95E+03 | HC: 1-102 (YYCSRWGGD.F) |

TABLE 9
Trastuzumab Cathepsin D digest assignments for the pH 7, 37° C., 2 days,
1:20 sample and analyzed by LC-MS on a QExactive™ EMR.

| | Expected | | |
|---|---|---|---|
| Mass | mass | Intensity | Assignment |
| 23439.60 | 23439.06 | 1.01E+05 | LC: 1-214 |
| 23556.53 | ND | 9.97E+04 | |
| 23458.24 | ND | 2.51E+04 | |
| 23335.81 | 2335.92 | 2.45E+04 | LC: 1-213 (...SSPVTKSFNRGE.C) |
| 15050.62 | 15050.77 | 1.68E+04 | HC: 1-140 (...PSSKSTSG) |
| 23745.59 | ND | 1.43E+04 | |
| 11198.24 | 11198.49 | 8.13E+03 | HC: 1-102 (YYCSRWGGD.F) |
| 23485.68 | 23486.34 | 6.99E+03 | HC: 2-222 (E.VQL...EPKS.C |
| 15098.40 | ND | 6.78E+03 | |
| 15366.71 | ND | 5.40E+03 | |
| 11246.75 | ND | 4.49E+03 | |
| 11276.68 | 11276.62 | 2.61E+03 | HC: 11-110 (G.LVQPGG...AMDYW.GQG |

TABLE 10
Peptides from the optimized trastuzumab digestion.^{a}

| | Expected | | |
|---|---|---|---|
| Mass | Mass | Intensity | Assignment |
| 11693.74 | 11694.06 | 4.24 x 10^{∧}5 | LC: 1-107 (...GTKVEIK.R) |
| 12121.26 | 12121.56 | 4.47 x 10^{∧}6 | LC: 1-111 (...VEIKRTVA.A) |
| 12488.77 | 12490.29 | 3.61 x 10^{∧}5 | HC: 241-349 |
| | | | (...G.GPSVFLFPP...KGQPREP.Q) |
| 12622.76 | 12623.14 | 2.24 x 10^{∧}5 | LC: 1-116 (...VAAPSVF.I) |
| 12688.71 | 12689.15 | 2.84 x 10^{∧}5 | HC: 1-115 (...DYWGQGTL.V) |
| 12832.74 | 12832.43 | 3.55 x 10^{∧}5 | G0F + HC: 243-341 |
| | | | (...S.VFLFPPKP...APIEKTISK.A) |
| 22132.34 | 22132.61 | 3.95 x 10^{∧}5 | LC: 1-202 (...EVTHQGLS.S) |
| 22517.13 | 22518.18 | 2.05 x 10^{∧}5 | 1 HC + 1 LC: |
| | | | HC: 132-223 |
| | | | (...L.APSSKSTS...KKVEPKSC.D) |
| | | | LC: 96-214 (...P.PTFGQG...NRGEC) |
| 47252.23 | 47252.72 | 4.66 x 10^{∧}3 | 1 LC + 1 HC: LC: 1-214 |
| | | | HC: 1-224 (...KKVEPKSCD.K) |
| 97630.79 | 97634.16 | 4.34 * 10^{∧}7 | 2LC + HC1 + HC2: LC: 1-214 |
| | | | HC1: 1-239 (...PAPELLG.G) |
| | | | HC2: 1-240 (...PAPELLGG.P) |

^{a}Assignments were made using a combination of antibody-specific rules (e.g. exclusion of NST containing sequence if no glycoforms present) and exact mass. All assignments were made within 2 Da for species < 50 kDa and within 4 Da for species <100 kDa.

TABLE 11
The deconvolved masses from the optimized trastuzimab cathepsin sample. The
data below was not searched for a sequence match, unless found in Table 5a.

| Masses | Masses | Masses | Masses | Masses | Masses | Masses |
|---|---|---|---|---|---|---|
| 4008.84 | 4443.69 | 4825.81 | 5061.71 | 12139.71 | 12692.24 | 23140.70 |
| 4089.76 | 4449.30 | 4826.75 | 5084.22 | 12143.32 | 12832.74 | 23283.26 |
| 4237.22 | 4449.33 | 4826.76 | 5150.84 | 12159.14 | 12850.62 | 23437.75 |
| 4246.23 | 4452.19 | 4832.25 | 5202.61 | 12174.76 | 12853.28 | 23475.41 |
| 4270.23 | 4461.79 | 4839.90 | 5217.75 | 12337.24 | 12994.16 | 24389.74 |
| 4291.69 | 4471.23 | 4848.55 | 5236.98 | 12375.13 | 13778.71 | 24551.80 |
| 4303.23 | 4483.79 | 4863.24 | 5274.23 | 12470.32 | 13833.24 | 96566.32 |
| 4339.22 | 4549.73 | 4884.13 | 5302.68 | 12488.77 | 13938.75 | 96925.26 |
| 4339.91 | 4556.75 | 4943.21 | 5436.20 | 12490.30 | 21911.25 | 97048.07 |
| 4356.71 | 4597.25 | 4970.83 | 5888.96 | 12622.71 | 22132.32 | 97076.31 |
| 4356.71 | 4611.53 | 4972.23 | 11452.17 | 12622.76 | 22153.26 | 97269.77 |
| 4356.72 | 4616.74 | 4972.24 | 11515.76 | 12632.39 | 22516.71 | 97454.93 |
| 4366.14 | 4632.06 | 4988.81 | 11693.74 | 12650.68 | 22536.58 | 97627.94 |
| 4366.27 | 4663.73 | 4988.86 | 11764.67 | 12652.70 | 22994.24 | 97735.70 |
| 4428.70 | 4680.65 | 5010.74 | 12104.66 | 12670.80 | 23082.16 | 97792.21 |
| 4428.72 | 4808.72 | 5012.61 | 12121.22 | 12688.75 | 23120.77 | 97856.71 |
| 4437.27 | | | | | | |

TABLE 12
Top down analysis of the 12.12 kDa peptide. Product ions and
their associated errors (all within 10 ppm) are listed.

| | | Theoretical | Observed | Mass Difference |
|---|---|---|---|---|
| Name | Ion Type | Mass | Mass | (ppm) |
| B6 | B-H2O | 698.3058 | 698.3069 | 1.53228 |
| B6 | B-neutral | 716.3163 | 716.3176 | 1.77296 |
| B7 | B-H2O | 785.3379 | 785.339 | 1.451605 |
| B8 | B-H2O | 882.3906 | 882.3912 | 0.657305 |
| B9 | B-neutral | 987.4332 | 987.4337 | 0.557 |
| B10 | B-H2O | 1056.455 | 1056.455 | 0.681525 |
| B10 | B-neutral | 1074.465 | 1074.467 | 1.321588 |
| B11 | B-H2O | 1169.539 | 1169.54 | 0.820836 |
| B11 | B-neutral | 1187.549 | 1187.551 | 1.145216 |
| B12 | B-neutral | 1274.581 | 1274.582 | 0.808109 |
| B13 | B-H2O | 1327.608 | 1327.609 | 0.768299 |
| B14 | B-neutral | 1432.65 | 1432.651 | 0.272223 |
| B16 | B-neutral | 1588.74 | 1588.741 | 0.26436 |
| B17 | B-neutral | 1703.767 | 1703.768 | 0.633889 |
| B18 | B-neutral | 1859.868 | 1859.868 | −0.01613 |
| B27 | B-neutral | 2818.346 | 2818.344 | −0.76463 |
| B28 | B-neutral | 2933.373 | 2933.371 | −0.85056 |
| B35 | B-neutral | 3674.754 | 3674.756 | 0.583713 |
| B39 | B-neutral | 4222.03 | 4222.031 | 0.37778 |
| B43 | B-H2O | 4557.225 | 4557.215 | −2.41265 |
| B44 | B-H2O | 4654.278 | 4654.288 | 2.115258 |
| B44 | B-neutral | 4672.289 | 4672.31 | 4.61123 |
| B69 | B-H2O | 7315.67 | 7315.652 | −2.42015 |
| B70 | B-neutral | 7448.707 | 7448.654 | −7.08109 |
| B89 | B-H2O | 9637.697 | 9637.704 | 0.692074 |
| B90 | B-H2O | 9765.756 | 9765.764 | 0.818165 |
| B90 | B-neutral | 9783.766 | 9783.75 | −1.65683 |
| B91 | B-neutral | 9920.825 | 9920.82 | −0.55641 |
| B94 | B-neutral | 10285.98 | 10285.97 | −1.49816 |
| B97 | B-H2O | 10563.13 | 10563.13 | 0.055855 |
| B98 | B-H2O | 10710.2 | 10710.22 | 2.145619 |
| B99 | B-neutral | 10785.23 | 10785.23 | 0.69725 |
| B102 | B-neutral | 11071.35 | 11071.39 | 3.432281 |
| B104 | B-neutral | 11298.52 | 11298.54 | 1.498427 |
| B109 | B-H2O | 11907.88 | 11907.92 | 3.496005 |
| Y6 | Y-H2O | 668.4334 | 668.434 | 0.905101 |
| Y6 | Y-neutral | 686.4439 | 686.4451 | 1.755424 |
| Y7 | Y-H2O | 797.476 | 797.4772 | 1.523557 |
| Y7 | Y-neutral | 815.4865 | 815.4878 | 1.612534 |
| Y8 | Y-H2O | 896.5444 | 896.5454 | 1.120971 |
| Y8 | Y-neutral | 914.5549 | 914.5562 | 1.426924 |
| Y9 | Y-H2O | 1024.639 | 1024.64 | 0.531894 |
| Y9 | Y-neutral | 1042.65 | 1042.651 | 1.194073 |
| Y10 | Y-H2O | 1125.687 | 1125.688 | 1.123758 |
| Y10 | Y-neutral | 1143.698 | 1143.699 | 1.368369 |
| Y11 | Y-H2O | 1182.708 | 1182.71 | 0.934296 |
| Y11 | Y-neutral | 1200.719 | 1200.72 | 1.086849 |
| Y12 | Y-H2O | 1310.767 | 1310.768 | 1.010858 |
| Y12 | Y-neutral | 1328.778 | 1328.779 | 0.997157 |
| Y13 | Y-H2O | 1367.789 | 1367.789 | 0.193743 |
| Y13 | Y-neutral | 1385.799 | 1385.8 | 0.76851 |
| Y14 | Y-H2O | 1514.857 | 1514.858 | 0.432384 |
| Y14 | Y-neutral | 1532.867 | 1532.869 | 0.75349 |
| Y15 | Y-H2O | 1615.905 | 1615.904 | −0.57243 |
| Y15 | Y-neutral | 1633.915 | 1633.916 | 0.413118 |
| Y16 | Y-H2O | 1712.957 | 1712.958 | 0.183892 |
| Y16 | Y-neutral | 1730.968 | 1730.969 | 0.528606 |
| Y17 | Y-H2O | 1810.01 | 1810.011 | 0.58287 |
| Y17 | Y-neutral | 1828.021 | 1828.022 | 0.467719 |
| Y18 | Y-neutral | 1929.068 | 1929.069 | 0.142556 |
| Y19 | Y-neutral | 2030.116 | 2030.116 | 0.145312 |
| Y20 | Y-neutral | 2193.179 | 2193.18 | 0.212021 |
| Y21 | Y-neutral | 2330.238 | 2330.239 | 0.281087 |
| Y22 | Y-neutral | 2458.297 | 2458.296 | −0.25424 |
| Y23 | Y-neutral | 2586.355 | 2586.353 | −1.04587 |
| Y27 | Y-neutral | 3115.531 | 3115.516 | −4.7857 |
| Y29 | Y-neutral | 3333.637 | 3333.633 | −1.23889 |
| Y30 | Y-neutral | 3448.664 | 3448.668 | 1.255559 |
| Y32 | Y-neutral | 3674.759 | 3674.756 | −0.68576 |
| Y37 | Y-NH3 | 4220.076 | 4220.11 | 8.025921 |
| Y41 | Y-neutral | 4665.298 | 4665.294 | −0.68806 |
| Y45 | Y-NH3 | 5042.452 | 5042.45 | −0.37085 |
| Y68 | Y-neutral | 7538.769 | 7538.774 | 0.642015 |
| Y72 | Y-H2O | 7873.964 | 7873.952 | −1.55576 |
| Y72 | Y-neutral | 7891.975 | 7891.978 | 0.335784 |
| Y95 | Y-H2O | 10507.25 | 10507.24 | −1.13017 |
| Y98 | Y-H2O | 10750.38 | 10750.32 | −5.55097 |
| Y99 | Y-neutral | 10839.42 | 10839.41 | −1.6869 |
| Y103 | Y-neutral | 11213.6 | 11213.6 | −0.00312 |
| Y104 | Y-NH3 | 11327.68 | 11327.63 | −4.73574 |

TABLE 13
Top down analysis of the 98 kPa peptide.
228 product ions and their associated errors (all within 10 ppm) are listed. Where specified,
a standard b/y fragment ion within the HC or LC was ID’d with a covalent modification,
corresponding to a cross-linked fragment.

| | | | Mass | | | | | |
|---|---|---|---|---|---|---|---|---|
| | | Theo- | Diff- | | Covalent | | Theo- | Mass |
| | Name | retical | erence | HC | modification | | retical | Diff- |
| Ion | and Chain | Mass | (ppm) | Ion | (LC seq.) | Sequence | Mass | erence |
| B5 | B-H2O, LC | 570.2473 | -0.61 | Y56 | C | HC, GG | 5988.9061 | -3.62 |
| B6 | B-H2O, LC | 698.3058 | 2.25 | Y218 | C | HC, G | 23298.3690 | 4.72 |
| B13 | B-H2O, LC | 1327.6079 | 0.39 | Y22 | CEG | HC, GG | 2564.0799 | -0.85 |
| B17 | B, LC | 1703.7672 | 1.10 | Y60 | CEG | HC, GG | 6595.1710 | 4.76 |
| B19 | B, LC | 1958.9367 | 3.66 | Y95 | CEG | HC, GG | 10268.9711 | -1.08 |
| B20 | B-H2O, LC | 2041.9739 | 6.70 | Y120 | CEG | HC, GG | 12569.1722 | 5.30 |
| B27 | B, LC | 2818.3464 | 4.95 | Y124 | CEG | HC, GG | 12943.3524 | -6.63 |
| B28 | B, LC | 2933.3733 | -0.20 | Y216 | CEG | HC, GG | 23398.3720 | 5.01 |
| B28 | B-H2O, LC | 2915.3628 | 0.00 | Y113 | CEGR | HC, GG | 12096.9553 | 3.60 |
| B28 | B, LC | 2933.3733 | -1.98 | Y120 | CEGR | HC, GG | 12725.2733 | -6.23 |
| B39 | B, LC | 4222.0297 | 3.17 | Y121 | CEGR | HC, GG | 12812.3054 | 5.97 |
| B39 | B, LC | 4222.0297 | 2.06 | Y122 | CEGR | HC, GG | 12899.3374 | -3.91 |
| B43 | B, LC | 4575.2360 | 0.77 | Y128 | CEGR | HC, GG | 13469.6751 | -9.07 |
| B60 | B-H2O, LC | 6380.2124 | -7.76 | Y118 | CEGRN | HC, GG | 12681.2470 | 5.63 |
| B70 | B-H2O, LC | 7430.6966 | 0.05 | Y122 | CEGRN | HC, GG | 13013.3802 | -5.76 |
| B74 | B-H2O, LC | 7892.9445 | 7.47 | Y107 | CEGRNFS | HC, GG | 11820.7356 | -1.11 |
| B79 | B, LC | 8439.2457 | -0.64 | Y114 | CEGRNFS | HC, GG | 12532.1311 | -8.91 |
| B82 | B, LC | 8780.3680 | 0.90 | Y33 | CEGRNFSK | HC, GG | 4421.1388 | 1.02 |
| B94 | B, LC | 10285.9840 | -1.46 | Y30 | CEGRNFSKTV | HC, GG | 4309.0746 | 3.54 |
| B94 | B-H2O, LC | 10267.9735 | 1.51 | Y24 | CEGRNFSKTV | HC, G | 3694.7722 | 6.10 |
| B100 | B, LC | 10913.2857 | 1.85 | Y108 | CEGRNFSKTV | HC, G | 12260.0146 | -6.60 |
| B100 | B, LC | 10913.2857 | -0.88 | Y106 | CEGRNFSKTVP | HC, GG | 12158.9670 | -9.37 |
| B103 | B-H2O, LC | 11181.4393 | 2.29 | Y207 | CEGRNFSKTVPS | HC, GG | 23389.4305 | 2.64 |
| B103 | B, LC | 11199.4498 | -1.05 | Y114 | CEGRNFSKTVPSS | HC, G | 13171.4898 | -8.11 |
| B105 | B, LC | 11427.5608 | -0.75 | Y52 | CEGRNFSKTVPSSL | HC, GG | 7005.4100 | 0.13 |
| B111 | B-H2O, LC | 12077.9836 | 3.13 | Y103 | CEGRNFSKTVPSSLGQ | HC, G | 12359.0467 | -8.04 |
| B112 | B, LC | 12167.0312 | 3.74 | Y99 | CEGRNFSKTVPSSLGQH | HC, G | 12163.9727 | -9.82 |
| B112 | B, LC | 12167.0312 | -0.09 | Y202 | CEGRNFSKTVPSSLGQH | HC, G | 23319.3738 | 3.64 |
| B114 | B, LC | 12351.1160 | -0.09 | Y110 | CEGRNFSKTVPSSLGQHT | HC, G | 13277.5389 | -7.68 |
| B115 | B, LC | 12450.1844 | 1.20 | Y108 | CEGRNFSKTVPSSLGQHT | HC, GG | 13053.3865 | -4.47 |
| B115 | B, LC | 12450.1844 | -0.18 | Y110 | CEGRNFSKTVPSSLGQHT | HC, GG | 13237.5076 | 2.57 |
| B116 | B, LC | 12597.2528 | 1.63 | Y121 | CEGRNFSKTVPSSLGQHT | HC, GG | 14296.0473 | -5.96 |
| B116 | B, LC | 12597.2528 | -0.15 | Y100 | CEGRNFSKTVPSSLGQHTV | HC, GG | 12420.1526 | 1.75 |
| B117 | B, LC | 12710.3369 | 0.63 | Y202 | CEGRNFSKTVPSSLGQHTV | HC, GG | 23419.4525 | -0.34 |
| B117 | B-H2O, LC | 12692.3264 | 2.65 | Y86 | CEGRNFSKTVPSSLGQHTVE | HC, G | 11155.4748 | -1.86 |
| B117 | B, LC | 12710.3369 | -0.22 | Y110 | CEGRNFSKTVPSSLGQHTVE | HC, G | 13505.6500 | -6.27 |
| B118 | B, LC | 12857.4053 | -0.45 | Y115 | CEGRNFSKTVPSSLGQHTVE | HC, G | 13992.8931 | -6.37 |
| B118 | B-H2O, LC | 12839.3948 | 2.63 | Y111 | CEGRNFSKTVPSSLGQHTVE | HC, GG | 13562.6715 | -6.47 |
| B118 | B, LC | 12857.4053 | -0.19 | Y111 | CEGRNFSKTVPSSLGQHTVE | HC, GG | 13562.6715 | -4.17 |
| B119 | B, LC | 12954.4580 | 1.30 | Y200 | CEGRNFSKTVPSSLGQHTVE | HC, GG | 23350.3568 | 4.95 |
| B122 | B-H2O, LC | 13235.5593 | -0.53 | | Covalent | | Theo- | Mass |
| | | | | LC | modification | | retical | Diff- |
| | | | | Ion | (HC seq.) | Sequence | Mass | erence |
| B122 | B, LC | 13253.5698 | -0.26 | Y40 | CDK | LC | 4738.2699 | 9.72 |
| B128 | B, LC | 13895.9035 | -8.40 | Y109 | CDKTH | LC | 12561.1425 | 6.68 |
| B211 | B, LC | 23117.4085 | -3.37 | Y203 | CDKTHTCPPC | LC | 23300.3100 | 5.69 |
| Y38 | Y, LC | 4211.0415 | 0.49 | Y200 | CDKTHTCPPCPA | LC | 23223.2987 | 4.83 |
| Y41 | Y-NH3, LC | 4464.1605 | 5.27 | Y201 | CDKTHTCPPCPAP | LC | 23407.3834 | 2.99 |
| Y46 | Y, LC | 5092.4546 | 3.52 | Y100 | CDKTHTCPPCPAPEL | LC | 12644.0125 | 7.82 |
| Y92 | Y-NH3, LC | 10135.8855 | 1.97 | Y196 | CDKTHTCPPCPAPELLGG | LC | 23380.3977 | 3.17 |
| Y96 | Y-NH3, LC | 10532.0500 | 2.68 | Y42 | CKPEV | LC | 5303.5446 | 8.35 |
| Y107 | Y-H2O, LC | 11736.7342 | -2.15 | Y115 | CS | LC | 12827.2903 | -7.31 |
| Y112 | Y, LC | 12352.1297 | 0.01 | Y99 | CSKP | LC | 11387.4808 | 0.33 |
| Y112 | Y-H2O, LC | 12334.1192 | 0.63 | Y210 | CSKP | LC | 23350.4551 | 0.74 |
| Y115 | Y, LC | 12638.2574 | -8.97 | Y114 | CSKPE | LC | 13053.4220 | -7.20 |
| Y116 | Y-NH3, LC | 12661.2497 | -6.34 | Y208 | CSKPE | LC | 23250.3914 | 1.70 |
| Y116 | Y-H2O, LC | 12677.2684 | 5.57 | Y209 | CSKPE | LC | 23378.4499 | -0.34 |
| Y120 | Y, LC | 13137.5005 | -7.36 | Y27 | CSKPEVKKD | LC | 4015.9579 | -6.65 |
| Y121 | Y, LC | 13238.5481 | -1.30 | Y98 | CSKPEVKKD | LC | 11839.7405 | -0.83 |
| Y126 | Y, LC | 13895.8352 | -3.49 | Y204 | CSKPEVKKD | LC | 23362.5281 | -3.67 |
| Y127 | Y-NH3, LC | 13963.8074 | 2.12 | Y7 | CSKPEVKKDV | LC | 1922.8973 | 9.26 |
| Y129 | Y-NH3, LC | 14289.9341 | 4.42 | Y95 | CSKPEVKKDVP | LC | 11709.6983 | 0.30 |
| B4 | B, HC_GG | 469.2536 | -2.43 | Y26 | CSKPEVKKDVPT | LC | 4216.0736 | -9.40 |
| B6 | B, HC_GG | 697.3646 | 1.52 | Y99 | CSKPEVKKDVPT | LC | 12315.0196 | 6.57 |
| B9 | B, HC_GG | 898.4396 | -7.45 | Y201 | CSKPEVKKDVPT | LC | 23419.5856 | -6.02 |
| B10 | B, HC_GG | 955.4611 | 2.88 | Y107 | CSKPEVKKDVPTNS | LC | 13297.5392 | -7.70 |
| B10 | B-H2O, HC_GG | 937.4506 | 0.91 | Y199 | CSKPEVKKDVPTNS | LC | 23434.5602 | -4.02 |
| B11 | B, HC_GG | 1068.5451 | 2.05 | Y96 | CSKPEVKKDVPTNSPK | LC | 12334.0214 | 7.38 |
| B12 | B, HC_GG | 1167.6135 | 1.52 | Y100 | CSKPEVKKDVPTNSPK | LC | 12840.3107 | 6.24 |
| B13 | B, HC_GG | 1295.6721 | -0.69 | Y91 | CSKPEVKKDVPTNSPKH | LC | 11945.8732 | -9.21 |
| B19 | B, HC_GG | 1862.9850 | 0.98 | Y94 | CSKPEVKKDVPTNSPKH | LC | 12276.9748 | -7.40 |
| B19 | B, HC_GG | 1862.9850 | -0.53 | Y96 | CSKPEVKKDVPTNSPKH | LC | 12471.0803 | 6.74 |
| B21 | B-H2O, HC_GG | 2045.0906 | 0.85 | Y194 | CSKPEVKKDVPTNSPKH | LC | 23268.5012 | -3.56 |
| B26 | B, HC_GG | 2451.2301 | 1.65 | Y94 | CSKPEVKKDVPTNSPKHN | LC | 12391.0177 | -4.33 |
| B27 | B-H2O, HC_GG | 2580.2881 | -7.35 | Y98 | CSKPEVKKDVPTNSPKHN | LC | 12845.2757 | -4.02 |

| B28 | B, HC_GG | 2712.3415 | -1.80 |
|---|---|---|---|
| B30 | B, HC_GG | 2953.5205 | 2.81 |
| B31 | B, HC_GG | 3068.5474 | -0.50 |
| B31 | B-H2O, HC_GG | 3050.5369 | -1.85 |
| B31 | B, HC_GG | 3068.5474 | -2.07 |
| B55 | B, HC_GG | 5905.0399 | 1.94 |
| B62 | B, HC_GG | 6731.4009 | -1.19 |
| B73 | B-H2O, HC_GG | 7875.0046 | -2.08 |
| B101 | B, HC_GG | 11058.4673 | -0.37 |
| B102 | B, HC_GG | 11173.4943 | -3.93 |
| B102 | B-H2O,HC_GG | 11155.4838 | -1.04 |
| B102 | B, HC_GG | 11173.4943 | -0.72 |
| B103 | B, HC_GG | 11230.5157 | -4.97 |
| B103 | B-H2O, HC_GG | 11212.5052 | -7.39 |
| B106 | B, HC_GG | 11611.6846 | -5.37 |
| B108 | B-H2O, HC_GG | 11839.7415 | 2.06 |
| B108 | B, HC_GG | 11857.7520 | -0.83 |
| B112 | B, HC_GG | 12391.9747 | 0.32 |
| B115 | B, HC_GG | 12663.1279 | -2.16 |
| B116 | B, HC_GG | 12762.1963 | 1.08 |
| B116 | B-H2O, HC_GG | 12744.1858 | 1.81 |
| B116 | B, HC_GG | 12762.1963 | -1.01 |
| B118 | B, HC_GG | 12962.3124 | -3.30 |
| B121 | B, HC_GG | 13207.4135 | 9.20 |
| B122 | B-H2O, HC_GG | 13276.4351 | 6.78 |
| B128 | B-H2O, HC_GG | 13845.7524 | -1.47 |
| B129 | B, HC_GG | 14010.8313 | 0.54 |
| B129 | B, HC_GG | 14010.8313 | -0.71 |
| B132 | B, HC_GG | 14292.0052 | -0.21 |
| B184 | B, HC_GG | 19530.5785 | 0.07 |
| B205 | B, HC_GG | 21665.6480 | 1.74 |
| Y9 | Y, HC_G | 894.4395 | 7.05 |
| Y9 | Y, HC_G | 894.4395 | 5.49 |
| Y24 | Y-NH3, HC_G | 2557.2253 | 6.48 |
| Y25 | Y, HC_GG | 2631.2733 | 4.53 |
| Y26 | Y, HC_G | 2788.3472 | 8.15 |
| Y27 | Y, HC_GG | 2845.3686 | 7.44 |
| Y28 | Y-H2O, HC_GG | 2955.4531 | -7.34 |
| Y29 | Y, HC_GG | 3074.5113 | 2.03 |
| Y29 | Y-H2O, HC_GG | 3056.5008 | -3.07 |
| Y30 | Y-H2O, HC_GG | 3170.5437 | 2.87 |
| Y30 | Y-NH3, HC_GG | 3171.5277 | 6.09 |
| Y31 | Y, HC_G | 3315.6175 | 7.71 |
| Y33 | Y-H2O, HC_GG | 3482.7235 | -7.97 |
| Y34 | Y, HC_G | 3694.8143 | -5.29 |
| Y39 | Y-NH3,HC_GG | 4163.0061 | 2.82 |
| Y41 | Y, HC_G | 4515.1807 | 3.99 |
| Y42 | Y, HC_G | 4616.2284 | 5.16 |
| Y44 | Y, HC_GG | 4730.2713 | 7.36 |
| Y46 | Y, HC_GG | 4930.3874 | 2.76 |
| Y50 | Y-H2O, HC_GG | 5282.5621 | 5.77 |
| Y50 | Y-NH3, HC_G | 5327.5723 | -0.25 |
| Y51 | Y-H2O, HC_GG | 5383.6098 | 3.68 |
| Y53 | Y-NH3, HC_GG | 5582.7306 | 0.39 |
| Y57 | Y, HC_G | 6079.9791 | -6.35 |
| Y57 | Y-NH3, HC_G | 6062.9526 | 7.01 |
| Y64 | Y-NH3, HC_G | 6747.3333 | 9.08 |
| Y69 | Y-NH3, HC_GG | 7220.5607 | -1.26 |
| Y69 | Y-NH3, HC_G | 7300.5981 | -2.50 |
| Y72 | Y, HC_G | 7560.7465 | -6.96 |
| Y106 | Y, HC_G | 10971.4043 | -7.57 |
| Y106 | Y-H2O, HC_GG | 10923.3833 | -5.52 |
| Y107 | Y, HC_GG | 11028.4258 | 1.10 |
| Y108 | Y-NH3, HC_G | 11122.4677 | -0.13 |
| Y109 | Y-H2O, HC_GG | 11178.5052 | -2.14 |
| Y113 | Y, HC_G | 11682.7999 | -6.94 |
| Y118 | Y-H2O, HC_GG | 12105.0277 | 0.67 |
| Y118 | Y-NH3, HC_G | 12136.0223 | 2.44 |
| Y122 | Y, HC_GG | 12455.1714 | 3.03 |
| Y123 | Y-H2O, HC_GG | 12536.2293 | 0.34 |
| Y123 | Y-H2O, HC_GG | 12536.2293 | 2.78 |
| Y123 | Y-H2O, HC_G | 12580.2555 | 1.03 |
| Y126 | Y-H2O, HC_G | 12893.4557 | -8.62 |

| | | | |
|---|---|---|---|
| Y132 | Y, HC_GG | 13559.7318 | -9.82 |
| Y132 | Y-H2O, HC_G | 13599.7267 | 1.15 |
| Y136 | Y-H2O, HC_GG | 14021.8891 | -6.62 |
| Y191 | Y-NH3, HC_GG | 20239.8092 | -2.50 |
| Y204 | Y, HC_GG | 21648.6030 | 3.70 |
| Y217 | Y-NH3, HC_G | 23178.3334 | 3.61 |
| Y217 | Y-NH3, HC_G | 23178.3334 | 5.04 |
| Y218 | Y-NH3, HC_G | 23280.3347 | 4.68 |
| Y218 | Y-H2O, HC_G | 23279.3507 | -0.68 |
| Y220 | Y-NH3, HC_GG | 23424.3882 | 1.93 |

TABLE 14
Amino acid sequences of the monoclonal antibodies and enzymes emploved in
the study

Rituximab (147,076.8 Da with pyro-Glu light and heavy chains, no C-terminal Lys, and
G0F/G0F)
Rituximab light chain (SEO ID NO: 1)
QIVLSQSPAILSASPGEKVTMTCRASSSVSYIHWFQQKPGSSPKPWIYATSNLASG
VPVRFSGSGSGTSYSLTISRVEAEDAATYYCQQWTSNPPTFGGGTKLEIKRTVAA
PSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
Rituximab heavy chain (SEO ID NO: 2)
QVQLQQPGAELVKPGASVKMSCKASGYTFTSYNMHWVKQTPGRGLEWIGAIYP
GNGDTSYNQKFKGKATLTADKSSSTAYMQLSSLTSEDSAVYYCARSTYYGGDW
YFNVWGAGTTVTVSAASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKAEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMH
EALHNHYTQKSLSLSPGK
Obinutuzumab (148,629.4 Da with pyro-Glu heavy chains, no C-terminal Lys, and G0/G0)
Obinutuzumab light chain (SEQ ID NO: 3)
DIVMTQTPLSLPVTPGEPASISCRSSKSLLHSNGITYLYWYLQKPGQSPQLLIYQM
SNLVSGVPDRFSGSGSGTDFTLKISRVEAEDVGVYYCAQNLELPYTFGGGTKVEI
KRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
Obinutuzumab heavy chain (SEQ ID NO: 4)
QVQLVQSGAEVKKPGSSVKVSCKASGYAFSYSWINWVRQAPGQGLEWMGRIFP
GDGDTDYNGKFKGRVTITADKSTSTAYMELSSLRSEDTAVYYCARNVFDGYWL
VYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHE
ALHNHYTQKSLSLSPGK
Eculizumab (147,871.4 Da with pyro-Glu heavy chains, no C-terminal Lys, and G0/G0)
Eculizumab light chain (SEQ ID NO: 5)
DIQMTQSPSSLSASVGDRVTITCGASENIYGALNWYQQKPGKAPKLLIYGATNL
ADGVPSRFSGSGSGTDFTLTISSLQPEDFATYYCQNVLNTPLTFGQGTKVEIKRTV
AAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Eculizumab heavy chain (SEQ ID NO: 6)
QVQLVQSGAEVKKPGASVKVSCKASGYIFSNYWIQWVRQAPGQGLEWMGEIL
PGSGSTEYTENFKDRVTMTRDTSTSTVYMELSSLRSEDTAVYYCARYFFGSSPN
WYFDVWGQGTLVTVSSASTKGPSVFPLAPCSRSTSESTAALGCLVKDYFPEPVT
VSWNSGALTSGVHTFPAVLQSSGLYSLSSVVTVPSSNFGTQTYTCNVDHKPSNT
KVDKTVERKCCVECPPCPAPPVAGPSVFLFPPKPKDTLMISRTPEVTCVVVDVSQ
EDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMH
EALHNHYTQKSLSLSLGK
Trastuzumab (147967.8 Da with pyro-Glu heavy chains, no C-terminal Lys, and G0/G0)
Trastuzumab Light Chain (SEQ IN NO: 7)
DIQMTQSPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKVEIKRTVA
APSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Trastuzumab Heavy Chain (SEO ID NO: 8)
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYA
MDYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMH
EALHNHYTQKSLSLSPGK
Human Cathepsin D: (SEQ ID NO: 9; 3.4.23.5 (BRENDA, IUBMB))
MQPSSLLPLALCLLAAPASALVRIPLHKFTSIRRTMSEVGGSVEDLIAKGPVSKYS
QAVPAVTEGPIPEVLKNYMDAQYYGEIGIGTPPQCFTVVFDTGSSNLWVPSIHCK
LLDIACWIHHKYNSDKSSTYVKNGTSFDIHYGSGSLSGYLSQDTVSVPCQSASSA
SALGGVKVERQVFGEATKQPGITFIAAKFDGILGMAYPRISVNNVLPVFDNLMQ
QKLVDQNIFSFYLSRDPDAQPGGELMLGGTDSKYYKGSLSYLNVTRKAYWQVH
LDQVEVASGLTLCKEGCEAIVDTGTSLMVGPVDEVRELQKAIGAVPLIQGEYMIP
CEKVSTLPAITLKLGGKGYKLSPEDYTLKVSQAGKTLCLSGFMGMDIPPPSGPLW
ILGDVFIGRYYTVFDRDNNRVGFAEAARL
Human Cathepsin L: (SEQ ID NO: 10; 3.4.22.15 (BRENDA, IUBMB))
MNPTLILAAFCLGIASATLTFDHSLEAQWTKWKAMHNRLYGMNEEGWRRAVW
EKNMKMIELHNQEYREGKHSFTMAMNAFGDMTSEEFRQVMNGFQNRKPRKGK
VFQEPLFYEAPRSVDWREKGYVTPVKNQGQCGSCWAFSATGALEGQMFRKTGR
LISLSEQNLVDCSGPQGNEGCNGGLMDYAFQYVQDNGGLDSEESYPYEATEESC
KYNPKYSVANDTGFVDIPKQEKALMKAVATVGPISVAIDAGHESFLFYKEGIYFE
PDCSSEDMDHGVLVVGYGFESTESDNNKYWLVKNSWGEEWGMGGYVKMAKD
RRNHCGIASAASYPTV

TABLE 15
Conditions tested for Cathepsin D/L activity
at a 1:200 mAb:enzyme ratio (wt:wt)

| | | | % | Time |
|---|---|---|---|---|
| ID | Temp | pH | MeOH | (Days) |
| A | RT | 7 | 0 | 2 |
| B | 37 | 3 | 0 | 2 |
| C | 50 | 3 | 0 | 2 |
| D | 37 | 5 | 0 | 2 |
| E | 50 | 5 | 0 | 2 |
| F | 37 | 7 | 10 | 2 |
| G | 37 | 7 | 30 | 2 |
| H | 37 | 7 | 50 | 2 |
| I | 37 | 5 | 50 | 2 |
| J | RT | 7 | 0 | 7 |

What is claimed is:

1. A method for cleaving an antibody, comprising mixing the antibody with cathepsin L, cathepsin D, or a combination of cathepsin L and cathepsin D to obtain one or more antibody fragments, wherein the antibody comprises a light chain comprising a light chain variable region (VL) and a light chain constant region (CL) and a heavy chain comprising a heavy chain variable region (VH) and a heavy chain constant region (CH), and wherein the cathepsin L and/or D cleaves the antibody between the VL and CL and/or between the VH and CH regions to create VL and/or VH antibody fragments and CL and/or CH antibody fragments; and optionally isolating one or more of the antibody fragments after the cleavage.

2. A method for analyzing the sequence of an antibody, comprising:
a. Cleaving the antibody with cathepsin L, cathepsin D, or a combination of cathepsin L and cathepsin D to obtain one or more antibody fragments, wherein the antibody comprises a light chain comprising a light chain variable region (VL) and a light chain constant region (CL) and a heavy chain comprising a heavy chain variable region (VH) and a heavy chain constant region (CH), and wherein the cathepsin L and/or D cleaves the antibody between the VL and CL and/or between the VH and CH regions to create VL and/or VH antibody fragments and CL and/or CH antibody fragments;
b. Optionally isolating one or more of the antibody fragments after the cleavage; and
c. Performing mass spectrometry (MS) analysis of the one or more antibody fragments.

3. The method of claim 1 or 2, wherein the antibody is an IgG antibody, such as a human IgG1, IgG2, IgG2A, IgG2B, or IgG4 antibody.

4. The method of claim 1, 2, or 3, wherein the cleavage generates VL and/or VH fragments.

5. The method of claim 4, wherein MS analysis is performed on the VL and/or VH fragments.

6. The method of any one of claims 1-5, wherein the heavy chain constant region comprises at least a CH1 region, and optionally further comprises a hinge, CH2 region, and/or CH3 region.

7. The method of any one of claims 1-6, wherein the antibody heavy chain constant region comprises at least a CH1, hinge, and CH2 region, and wherein the cathepsin L and/or cathepsin D further cleaves the antibody between the CH1 region and the hinge.

8. The method of any one of claims 1-7, wherein the antibody is cleaved with a combination of cathepsin L and cathepsin D.

9. The method of claim 8, wherein the method comprises incubating the antibody simultaneously with both cathepsin L and cathepsin D.

10. The method of any one of claims 1-9, wherein cleavage is conducted so as to achieve at least 50%, at least 60%, at least 70%, at least 80%, at least 85%, at least 90%, at least 95%, at least 98%, or 100% cleavage between the VL and CL and between the VH and CH regions.

11. The method of any one of claims 1-10, wherein the cleavage is conducted so as to achieve at least 50%, at least 60%, at least 70%, at least 80%, at least 85%, at least 90%, at least 95%, at least 98%, or 100% cleavage at or below the hinge.

12. The method of any one of claims 1-11, wherein the cleavage is conducted at a cathepsin L and/or cathepsin D to antibody ratio of: 1:20 to 1:2000, 1:20 to 1:500, 1:50 to 1:500, 1:100 to 1:500, 1:200 to 1:1000, 1:200 to 1:2000, 1:500 to 1:2000, 1:1000 to 1:2000, or 1:20, 1:50, 1:100, 1:200, 1:300, 1:400, 1:500, or 1:1000.

13. The method of any one of claims 1-12, wherein the antibody is an IgG antibody and is cleaved with a combination of cathepsin L and cathepsin D, wherein the cleavage results in VL, VH, CL, CH1, CH2, CH3, CH2-CH3, CL+CH1 (bonded), F(ab′), and F(ab′)2 fragments.

14. The method of any one of claims 1-13, wherein cleavage is conducted by incubating the antibody with the cathepsin L, cathepsin D, or combination of cathepsin D and L at pH 2-8 (such as pH 2-7, pH 2-6, pH 3-6, pH 3-5, pH 2, pH 2.5, pH 3, pH 3.5, pH 4, pH 4.5, pH 5, pH 5.5, pH 6, pH 7, or pH 8), at a temperature from room temperature to 50° C., and in the presence of no more than 50% organic solvent (e.g. acetonitrile, methanol, ethanol, or isopropyl alcohol), wherein the antibody is in a native state.

15. The method of claim 14, wherein the pH is from 3 to 5, such as 3, 3.5, 4, 4.5, or 5.

16. The method of claim 14, wherein the pH is from 3.5 to 4.5.

17. The method of claim 14, wherein the pH is 4.

18. The method of any one of claims 14-17, wherein no more than 30% organic solvent is present.

19. The method of claims 14-18, wherein cleavage is conducted in the presence of 10-30% organic solvent (e.g. 10-30% acetonitrile, 10-30% methanol, 10-30% ethanol, or 10-30% isopropyl alcohol).

20. The method of any one of claims 14-19, wherein no more than 10% organic solvent is present.

21. The method of any one of claims 14-20, wherein the temperature is between 37° C. and 50° C.

22. A method for analyzing the sequence of an antibody, comprising:
a. Cleaving the antibody with a combination of cathepsin L and cathepsin D to obtain one or more antibody fragments that comprise at least a light chain variable region (VL) fragment and/or a heavy chain variable region (VH) fragment,
i. wherein the antibody is in a native state;
ii. wherein the antibody comprises a light chain comprising a light chain variable region (VL) and a light chain constant region (CL) and a heavy chain comprising a heavy chain variable region (VH) and a heavy chain constant region (CH), and wherein the cathepsin L and D cleave the antibody between the VL and CL and/or between the VH and CH regions to create VL and/or VH antibody fragments and CL and/or CH antibody fragments, and
iii. wherein the cleavage is performed at a temperature between 25° C. and 50° C. and a pH from 3 to 5, and in the presence of no more than 30% organic solvent;
b. Optionally isolating one or more of the antibody fragments after the cleavage; and
c. Performing mass spectrometry (MS) analysis of the one or more antibody fragments.

23. The method of claim 22, wherein the cleavage is conducted at a cathepsin L and/or cathepsin D to antibody ratio of: 1:20 to 1:2000, 1:20 to 1:500, 1:50 to 1:500, 1:100 to 1:500, 1:200 to 1:1000, 1:200 to 1:2000, 1:500 to 1:2000, 1:1000 to 1:2000, or 1:20, 1:50, 1:100, 1:200, 1:300, 1:400, 1:500, or 1:1000.

24. The method of any one of claims 1-23, wherein, following cleavage, the one or more antibody fragments are subjected to one or more of buffer exchange, chromatography (e.g. liquid chromatography such as high performance liquid chromatography, or capillary electrophoresis), filtration (e.g. molecular weight cut-off filtration), reduction of disulfide bonds, exposure to guanidine hydrochloride, or alkylation.

25. The method of any one of claims 1-24, wherein the one or more antibody fragments following cleavage are isolated by chromatography or filtration.

26. The method of claim 25, wherein the one or more antibody fragments are isolated by liquid chromatography.

27. The method of any one of claims 2-26, wherein the mass spectrometry (MS) comprises LC-MS or LC-MS/MS.

28. The method of any one of claims 1-24, wherein the one or more antibody fragments are not isolated following cleavage.

29. The method of any one of claims 2-28, wherein the mass spectrometry comprises direct infusion mass spectrometry (DIMS), static spray infusion mass spectrometry, or flow injection mass spectrometry.

30. The method of any one of claims 2-29, wherein the mass spectrometry data is used to determine the amino acid sequence of at least a 10 amino acid stretch of one antibody fragment.

31. The method of claim 30, wherein the amino acid sequence of at least a 15 amino acid stretch of one antibody fragment is determined.

32. The method of claim 31, wherein the amino acid sequence of at least a 20 amino acid stretch of one antibody fragment is determined.

33. The method of any one of claims30-32, wherein the amino acid sequence of at least one antibody CDR region is determined.

34. The method of claim 33, wherein the sequence of the VH and/or VL CDR1, CDR2, and CDR3 is determined.

35. The method of any one of claims 2-34, wherein the complete amino acid sequence of at least one antibody fragment, such as a VH and/or VL fragment, is determined.

36. The method of any one of claims 30-35, wherein the sequence is determined by top-down analysis.

37. The method of claim 36, wherein the sequence is further analyzed or is confirmed by bottom-up analysis.

38. The method of any one of claims 1-37, wherein the amino acid sequence of the antibody VH and/or VL regions is unknown.

39. The method of any one of claims 1-37, wherein the amino acid sequence of the antibody is unknown.

40. The method of any one of claims 2-39, comprising performing MS on the CL and/or CH regions of the antibody, such as on a CL, CH1, CH2, and/or CH3 fragment generated from the cleavage.

41. The method of any one of claims 1-40, further comprising performing Edman degradation on at least one antibody fragment.

42. The method of any one of claims 1-41, wherein the antibody remains in the native state after the cleavage.

43. The method of any one of claims 1-42, wherein the antibody is not treated with denaturing agents or agents that reduce disulfide bonds during or after the cleavage.

44. The method of any one of claims 1-43, wherein the antibody retains its disulfide bonding during and after the cleavage.

45. The method of any one of claims 1-44, comprising performing mass spectrometry (MS) analysis of one or more antibody fragments following the cleavage, wherein the antibody remains in the native state and is not treated with denaturing agents or agents that reduce disulfide bonds prior to the MS analysis.

46. A composition comprising antibody fragments produced according to the method of any one of claims 1-45.

47. A composition comprising IgG antibody fragments produced from cleavage with cathepsin L, cathepsin D, or a combination of cathepsin L and D, wherein the antibody fragments comprise one or both of VH and VL fragments, and at least one, at least two, or at least three of the following fragments: CL, CH1, CH2, CH3, CH2-CH3, CL+CH1 (bonded), F(ab′), and F(ab′)2.

48. The composition of claim 47, wherein the VH and/or VL fragments comprise from 90 to 150 amino acids in length, such as from 95 to 140 amino acids, such as 100 to 140 amino acids, or such as from 100 to 120 amino acids.

49. The composition of claim 47, wherein the VH and/or VL fragments have a molecular mass of 10-16 kDa, such as 10-13 kDa, such as 10-12 kDa, such as 10-11 kDa, or such as 11-12 kDa.

50. A kit for digesting a protein with cathepsin L, cathepsin D, or a combination of cathepsin L and cathepsin D, the kit comprising (a) cathepsin L and/or cathepsin D; (b) one or more reaction buffers; and optionally (c) instructions for use in digesting proteins.

51. The kit of claim 50, wherein the kit is capable of cleaving an antibody according to the method of any one of claims 1-45.

52. The kit of claim 50 or 51, wherein the reaction buffer is at pH 2-8, pH 2-7, pH 2-6, pH 2-5, pH 3-6, pH 3-5, pH 3-4, pH 4-5, pH 2, pH 2.5, pH 3, pH 3.5, pH 4, pH 4.5, pH 5, pH 5.5, pH 6, pH 7, or pH 8; and optionally wherein the reaction buffer comprises one or more organic solvents (e.g. methanol, ethanol, isopropyl alcohol, or acetonitrile) at a concentration of 0-50%, 5-50%, 5-30%, 0-30%, 10-30%, 0-10%, 5-15%, 10-20%, 15-25%, or 20-30%.

53. The kit of claim 50, 51, or 52, wherein the reaction buffer is at pH 3, 3.5, 4, 4.5, or 5.

54. The kit of claim 53, wherein the reaction buffer is at pH 4.
